(19) United States
(12) Patent Application Publication
(10) Pub. No.: US 2006/0253924 A1
(43) Pub. Date: Nov. 9, 2006

(54) Nucleotide sequences involved in disease resistance

(75) Inventors: Stefan Cornelis Hendrikus Jozef Turk, Leidschendam (NL); Franciscus Lambertus Wilhelmus Takken, Amsterdam (NL); Camiel Frido De Jong, Wageningen (NL); Matthieu Henri Antoon Jozef Joosten, Wageningen (NL); Peter Jozef Gerard Marie De Wit, Rhenen (NL)

Correspondence Address: MCKENNA LONG & ALDRIDGE LLP, 1900 K STREET, NW, WASHINGTON, DC, 20006, US

(73) Assignee: Keygene N., V., AE Wageningen (NL)

(21) Appl. No.: 10/481,335

(22) PCT Filed: Jun. 24, 2002
(86) PCT No.: PCT/NL02/00417

§ 371 Date: Jun. 17, 2004

(30) Foreign Application Priority Data
Jun. 22, 2001 (EP) 01202420.4

(51) Int. Cl. A01H 1/00; C07H 21/04; C12N 1/20; C12N 15/82; C12N 5/04

(52) U.S. Cl. 800/279; 435/419; 435/468; 530/370; 536/23.6; 800/294; 435/252.2

(57) ABSTRACT

Method for the identification and isolation of expressed nucleic acid sequences that are associated with disease resistance of a plant against a plant pathogen, isolated nucleic acid molecules and variants and/or homologues thereof, constructs, plants and polypeptides encoded by said nucleotide sequences and the use of these sequences in the development of resistance in plants against pathogens.

### FIELD OF THE INVENTION

The present invention relates to the isolation and characterisation of nucleotide sequences that can be used to confer desired properties to a plant or plant cell. In particular the invention relates to nucleotide sequences involved in resistance of a plant against a pathogen.

### BACKGROUND OF THE INVENTION

Plants are under continuous attack of a wide variety of taxonomically very different pathogens such as bacteria, viruses, fungi, nematodes, aphids, insects and other pests. Most of these encounters between a pathogen and a plant stop at passive defence lines such as wax layers, cell walls and chemical barriers. However, when this primary barrier is overcome, the interaction between the plant and the pathogen can result in an invasion by the pathogen. This invasion may result in disease symptoms that can often lead to damage to the plant or even to death of the infected plant. The pathogen invasion may also lead to the infection of only a limited area of the plant or even only a few cells. To limit pathogen infections plants have developed a second line of defence. To this second line of defence a wide variety of highly complex and not yet elucidated mechanisms lay afoot. The basic principle resides in the mounting of the defence line by proteins derived from the transcription of specific resistance genes (R-genes). Takken et al. in the European Journal of Plant Pathology 106:699-713 (2000) recognises four different fundamental mechanisms: i). the product encoded by the R-gene inactivates a toxin which is produced by the pathogen and which toxin normally induces necrosis or inhibits the induction of other active defence responses; ii). the R-gene products encodes a pathogenicity target such that in absence of this target, plant resistance occurs; iii). the R-gene product primes the plant defence responses; and iv). the R-gene product mediates specific recognition of a specific pathogen that expresses a matching avirulence (Avr) gene (i.e. gene-for-gene resistance). Specific recognition of a pathogen derived Avr gene product, a so-called elicitor, by the host activates a signal transduction cascade that involves protein phosphorylation, ion fluxes, generation of reactive oxygen species and other signals. These signals subsequently trigger transcription of plant defence genes encoding proteins such as glutathione S-transferases, peroxidases, cell wall proteins, proteinase inhibitors, hydrolytic enzymes, pathogenesis related (PR) proteins and enzymes involved in secondary metabolism. In addition thereto, plant cells that are in direct contact with the invading pathogen die. This phenomenon is referred to as the hypersensitive response (HR). The HR is regarded as the hallmark of the gene-for-gene resistance principle. HR is an active process that requires protein syntheses and is often correlated with the induction of resistance in non-inoculated parts of the plant. This systemic acquired resistance (SAR) results in a significant reduction of disease symptoms caused by many pathogens.

The effect of a pathogen on a plant and on the economics of crop production can be very serious. Especially in the case of agronomically important crops such as tomato, potato, rice and corn this effect can be enormous if not disastrous. Accordingly there is a need for plants that are less vulnerable to pathogens and/or to plants that are more capable of defending themselves against invading pathogens.

In order to create such plants, whether transgenic or not, a further understanding of the underlying biochemistry and genetics of pathogen resistance is needed, and a further identification of components of the defence system of the plant is needed. To this end the pathway leading to a defence response in a plant has been studied by using a model system. In the course of these studies a large number of components that are involved in the induction of a defence response to a pathogen have been identified.

It is one of the goals of the present invention to provide for durable and/or broad resistance in crops and/or plants. It is a further goal of the present invention to identify nucleotide sequences and/or polypeptides that are related to, or capable of controlling, inducing, contributing or otherwise exercise a negative or positive influence or stimulus to, the signal transduction pathway. It is also a goal of the present invention to provide for methods, compounds or compositions that find use and/or application in the activation or regulation (up or down) of the defense mechanism of plants and in particular of the defense mechanism of plants against pathogens, more in particular the HR. It is further a goal of the present invention to identify and/or provide for downstream signaling components that are related to or are involved in the regulation of resistance, preferably race-specific resistance. It is a further goal of the present invention to identify and/or provide for promoters that drive pathogen inducible expression. Other goals of the present invention will become apparent from the description of the invention and its embodiments.

### DESCRIPTION OF THE INVENTION

The present invention relates to the isolation and characterisation of nucleotide sequences that are involved in the defence mechanism of a plant against a plant pathogen. More in particular, the invention relates to the isolation and characterisation of nucleotide sequences that can be used to confer upon a plant one or more desired and/or favourable properties relating to the defence mechanism of a plant against a plant pathogen.

A. Methods for the Identification and Isolation of Differentially Expressed Nucleic Acid Fragments

In a first major aspect the invention relates to a method for the identification and isolation of expressed nucleic acid fragments that are associated with pathogen resistance. For this purpose, expression profiles of at least two plants that differ in at least one property relating to disease resistance. The plants that differ are preferably of the same genus. More preferably the plants that differ are (genotypically) identical but placed in different conditions or the plants are isogenic except for a genetic determinant involved in, or associated with disease resistance. In particular, the method may be used to identify expressed nucleic acid fragments that are part of transcripts of genes that are associated with disease resistance in plants against pathogens. The corresponding genes may subsequently be isolated and used to confer advantageous properties relating to disease resistance to a plant. The expressed fragments may e.g. be provided in the form of a library consisting of nucleic acid sequences or fragments, and may in particular be in the form of one or more (amplified) restriction fragments, e.g. obtained through cDNA AFLP® as described in more detail hereinbelow.

Preferably, the method for identification and isolation of expressed nucleic acid fragments that are associated with disease resistance in a plant against a pathogen comprises at least the steps of:
(a) providing a first plant material;
(b) providing at least a second plant material, which differs from the first plant material in a property relating to disease resistance;
(c) optionally providing an additional plant material, which also differs from the first plant material in a property relating to disease resistance;
(d) comparing a transcript profile of the first plant material with a transcript profile of the second plant material, and optionally with a transcript profile of the additional plant material;
(e) identifying in the transcript profiles a nucleic acid fragment that corresponds to transcript that is differentially expressed between the first plant material and the second plant material, and optionally between the first plant material and the additional plant material.

In the method of the invention, the property relating to disease resistance preferably is a property associated with the HR in a plant, such as e.g. the presence or absence of the HR, or time after induction of the HR.

The plant materials to be used in the method of the invention, i.e. the first, second and additional plant materials, are preferably obtained from the same genus, same species, same variety, same cultivar or from the same plant. Yet the first plant material differs from the second and additional plant materials in at least one property relating to disease resistance. Such a difference may be the result of a genotypic difference: e.g. the second and additional plant materials may be obtained from plants that are mutants of the plant from which the first material is obtained. In such instances, the plants are preferably isogenic except for the genotypic difference. Alternatively, the plant materials are obtained from isogenic plants whereby the difference is the result of different tissues, different developmental stages or different conditions to which the plants have been subjected.

Preferably, the first plant material and the second plant material will be selected such that the desired property or properties of disease resistance is improved (e.g. increased) in the second plant material compared to the first plant material (although the invention in its broadest sense is not limited thereto). For instance, the first plant material may be a plant (material) with a normal or wild-type level of a property relating to disease resistance—i.e. used as a reference—in which case the second plant material may be a plant (material) with abnormal level of the property, i.e. improved, increased, or decreased. Optionally, the transcription profile of the first plant material—and optionally also of the second plant material—may be further compared with the transcription profile(s) of one or more additional plant materials, e.g. a third, a fourth and further plant materials. Preferably, any such additional plant materials are selected such that they differ from the first plant material in a property relating to disease resistance. They are preferably further selected such that they also differ from the second plant material in a property relating to disease resistance (e.g. the same as above), and/or differ from each other in said property. For example, a series or collection of plant materials may be ranked according to the level or degree of a property relating to disease resistance, upon which the plant material with the lowest level or degree of the property relating to disease resistance is used as the first plant material, and the remaining plant materials are used as the second plant material and additional plant materials (e.g. ranked according to the increase in the property or properties). Alternatively, from a collection of plant materials, the plant material with the highest level or degree of a property relating to disease resistance may be compared with the plant material with the lowest level or degree of the property or properties, in which the plant material with the highest level of the property or properties will generally be used as the second plant material and the plant material with the lowest level of the property or properties will generally be used as the first plant material. Thereafter, the first plant material and/or second plant material may optionally be further compared with one or more of the additional plant materials from the collection. With respect the above, it will be clear to the skilled person that, instead of the first plant material, also a known reference plant or plant material and/or data for such a known reference plant or plant material may be used.

Preferably, in the method of the invention, the first plant material is obtained from a plant that does not produce the HR and the second and optional additional plant material are obtained from one or more plants in which the HR has been induced. Preferably, the induction of the HR is suppressible, such that the second and optional additional plants may be grown under conditions suppressing the HR to a desired stage after which the suppressing conditions are removed to allow for induction of the HR. The suppression of the HR is preferably conveniently controlled by temperature. In a preferred embodiment of the invention, the second and additional plant materials have been obtained at different time intervals after induction of the HR. E.g., the second and additional plant materials may have been obtained at 0, 30, 60 and 90 minutes, from the onset of the HR. In a particular embodiment, the first plant material may be the plant material obtained at the onset of the HR, i.e. t=0, to be compared with the transcript profiles obtained from the materials obtained at the later timepoints.

In the method of the invention, the first plant material is preferably obtained from a plant that does not comprise a plant-pathogen derived avirulence (trans)gene. Avirulence genes are part of the gene-for-gene resistance mechanism through which plants, harbouring a specific resistance (R) gene, recognise pathogens carrying matching avirulence genes.

In the method of the invention, the second and optional additional plant material are preferably obtained from transgenic plants comprising a matching pair of (a) a plant pathogen derived avirulence gene, and (b) a plant resistance gene, whereby the matching pair of the resistance and avirulence genes is capable of inducing the HR. A range of matching pairs of resistance and avirulence genes are known in the art for a variety of plant species and their pathogens as reviewed by, and listed in Table 1, of Takken et al. (supra), which is incorporated by reference herein. Preferably, the plant pathogen providing the avirulence gene is a fungus, more preferably the fungus is Cladosporium fulvum. In a most preferred embodiment, the matching pair of the plant pathogen derived avirulence gene and the matching plant resistance gene is selected from the group consisting of the pairs Avr2 and Cf-2; Avr4 and Cf-4; Hcr9-4E and Cf-4E; Avr5 and Cf-5; and, Avr9 and Cf-9.

Generally, the plant materials used for generating the transcription profile(s) may be selected from one or more entire plants and/or from one or more parts, tissues and/or organs from one or more plants, including but not limited to the roots, stems, stalks, leaves, petals, fruits, seeds, tubers, meristems, sepals, and flowers as long as the plants or plant materials selected at least differ in a property relating disease resistance. Also, the plant materials selected may be representative for different stages of development of a plant, for different stages of a disease that may affect a plant, for plants that are exposed to different levels of stress (e.g. due to lack of water, light, nutrients, damage, etc.); again as long as the plant materials selected at least differ in a property relating to disease resistance. Usually, when the expression profiles of two or more such different plants are compared, the expression profiles of essentially same part(s), tissue(s) or organ(s) of such different plants will be compared.

When the expression profiles of two or more such different plants are compared, these plants will usually belong at least to the same genus, and preferably to the same species. E.g. plants from different varieties or lines showing differences in a property relating to disease resistance may be compared such as e.g. varieties or mutants with different properties relating to resistance, or any of these mutations in heterozygotes, homozygotes or hybrids.

Also, the plant materials may be two or more different parts, tissues and/or organs of a plant, which parts, tissues or organs differ in a property relating to disease resistance. Preferably, when the expression profiles of two or more such different parts, tissues or organs are compared, these parts, tissues or organs will be derived from plants belonging to the same species, more preferably from plants belonging to the same line or variety, and most preferably from the same plant or isogenic plants.

In the method of the invention preferably “comparable” expression profiles will be generated from each of the respective plant materials, by which is generally meant that these expression profiles should be such that they allow for the detection and identification of one or more differentially expressed fragments as outlined above. Generally, this means that the expression profiles will be obtained using the same technique and usually also using the same conditions. For instance, when cDNA AFLP® is used to generate such “comparable” expression profiles, such cDNA AFLP® will usually be carried out using the same restriction enzymes, adapters, primers, detection technique(s) and further conditions for essentially all plant materials used. However, the skilled person will understand that also several different comparable expression profiles may be generated from of the respective plant materials—e.g. using different restriction enzymes, different primer combinations (e.g. with different selective nucleotides), etc.—to provide several sets of expressed fragments.

For obtaining the transcript profiles that are compared in step (d), any technique known per se for transcript profiling may be used, and in particular any technique known per se that allows for the detection of one or more differences in expression between the respective plant materials. More in particular, a technique will be used that allows for the detection of differences in the transcripts or in the transcript levels as they are present in the respective plant materials, i.e. differences in mRNAs, mRNA levels or cDNAs corresponding to such mRNAs. Accordingly, the technique used for generating the transcript profiles will usually be such that the one or more expressed fragments are detected and subsequently identified that can be considered representative for one or more transcripts that are differentially expressed in the respective plant materials; preferably the levels of the expressed fragments as detected in the technique is such that they are representative for the levels the corresponding transcripts in the respective plant materials. Usually, these expressed fragments will be detected as, or will be identified by means of differences in the detection signals of the individual expressed fragments, e.g. bands, between the expression profiles generated from the respective plant materials. Of particular interest will be those markers the signal of which changes when the property or properties of disease resistance that are of interest (e.g. those mentioned above) changes (e.g. increases), for instance from the first plant material to the second plant material and/or from the first plant material to the additional plant materials, as further described below. Thus, for example, the expressed fragment's detectable signal—e.g. a band on a gel or autoradiograph, or a peak in a chromatogram—as present in the transcript profile(s) generated for the second plant material—and/or for any of the additional plant materials—may be absent or present at reduced levels in transcript profile(s) generated for the first plant material. The signals of the expressed fragments may also be increased—e.g. more intense—in the expression profile(s) generated for the second plant material—and/or for any of the additional plant materials—compared to the expression profile(s) generated for the first plant material. However, generally speaking the invention generally comprises the selection of any expressed fragment that is representative for any difference(s) in expression between the first plant material and the second plant material (and/or any of the additional plant materials). Thus, e.g. the invention also comprises (the selection of) fragments the signal of which changes (e.g. increases) when the property or properties of disease resistance that are of interest (e.g. those mentioned above) changes (e.g. increases), e.g. from the first plant material to the second plant material and/or from the first plant material to the additional plant materials. The invention also for instance comprises markers that are only associated with disease resistance during a certain—e.g. specific and/or limited—period of time, e.g. during development of the plant, during the development of the pathogen after invasion of the plant by the pathogen (e.g. during the development or activation of the defence response system of the plant or after the plant has been harvested.

The technique used for obtaining the transcription profiles is preferably such that the expressed fragments that are representative for the differentially expressed transcripts are provided in the form of nucleic acid fragment, such as amplified restriction fragments, that may be subjected to sequence analysis so as to provide the corresponding nucleic acid sequence. Such transcript-derived nucleotide sequences will be referred to herein as “expressed nucleic acid fragments”.

When the one or more fragments are generated in the form of such expressed fragments, the method of the invention may also comprise the optional further step of:

(f) isolating, purifying and/or sequencing the expressed fragment identified in step (e).

In particular, such expressed fragments may be (provided as) nucleotide sequences that are part of the differentially expressed transcripts, including e.g. restriction and/or PCR fragments derived from such transcripts.

Preferably, the expressed fragments are obtained by means of cDNA AFLP®, as further described below. In such a case, the expressed fragments will be provided as amplified restriction fragments—e.g. cDNA AFLP® fragments—that are identified as in bands with different intensities in the DNA fingerprints generated from the respective plant materials using cDNA AFLP®. Even more preferred, the transcript profiles may be generated by means of cDNA AFLP® carried out essentially as described hereinbelow, e.g. using the restriction enzymes, adapters, primers and further conditions as described below. The cDNA AFLP®—technique to be used preferably for the generation of the transcript profiles from the respective plant materials is based on the AFLP® method described by Vos et al. (1995, Nucl. Acids Res., 23: 4407-4414), whereby the starting DNA is a mixture of cDNA's obtained in the conventional manner by converting the mRNA pool isolated from the plant material of interest into a mixture of cDNA's using a reverse transcriptase. Total RNA extracted from the plant material may be used as template for the reverse transcriptase reaction but preferably the RNA template consists of poly-A+ enriched RNA obtained e.g. by oligo-dT chromatography.

The principle steps of the AFLP® method are as follows: a starting DNA (which is a mixture of cDNA's in the case of cDNA AFLP®) is digested with restriction enzymes. Preferably a combination of (1) a rare-cutting enzyme, e.g. a “six-cutter” such as EcoRI, and (2) a frequent-cutting enzyme, e.g. a “four-cutter” such as MseI, is used. Alternatively, a combination of two (different) four-cutters can be used. Oligonucleotide adapters are ligated onto the ends of the restriction fragments thus obtained. A pre-amplification PCR is then performed such that only the fragments with a rare-cutter-site (EcoRI) on one end and a frequent-cutter-site (MseI) on the other end are amplified. In the case of two four-cutters, the fragments that have two different end are preferentially amplified. The product of the pre-amplification is then used as template in a second and selective amplification under stringent conditions. The primers used in the selective amplification are complementary to the oligonucleotide adapters and in addition comprise at their 3′-ends arbitrarily chosen selective nucleotides which allow to amplify only a subset of the fragments amplified in the pre-amplification. The selective primers will usually contain between 1 and 5 selective nucleotides at their 3′-ends. Under these conditions only those fragments the ends of which are perfectly complementary to the selective primers will be amplified. E.g. two selective primers with each 1 selective nucleotide at their 3′-ends will amplify 1 out of 16 fragments, whereas two selective primers with each 2 selective nucleotide at their 3′-ends will amplify 1 out of 256 fragments. The amplified fragments may subsequently be separated by e.g. gel-electrophoresis, preferably on polyacrylamide gels and visualised by methods known per se. Each fragment may be defined by its size, i.e. length and by the combination of selective primers that have allowed it to be amplified. As mentioned, in the case of cDNA AFLP®, the starting DNA is cDNA obtained by reverse transcription of a mRNA population to be analysed. After pre-amplification and selective amplification the obtained fragments are derived from expressed transcripts and therefore herein referred to as “expressed fragments”. The intensity of the “expressed fragment” on the gel allows to evaluate the expression level of the corresponding transcript in the RNA population. Thus, cDNA AFLP®-technique can be used to provide amplified restriction fragments that have been derived from—or that, more generally, that correspond to at least part of—the transcripts that are present in the respective plant materials. Advantageously, in the cDNA AFLP®-technique, the intensity of a particular amplified restriction fragments as obtained is generally a measure for the level of the corresponding transcript in the plant material. Thus, for instance, when the transcript profiles of two or more plant materials are to be compared, expressed fragments useful in the invention may be conveniently selected on the basis of any differences in the intensities between corresponding band(s) in the respective cDNA AFLP® fingerprints generated for each of the respective plant materials. Also, the actual DNA fragments corresponding to the expressed fragments may conveniently by obtained e.g. by cutting the bands from the gel and subsequently isolating and purifying excised fragments. Thereupon, the fragments may optionally be cloned, re-amplified and/or sequenced.

The method described above may be used to provide a fragment, and usually a collection of at least two fragments, that are associated with/representative for differences in expression between the respective plant materials. In a preferred embodiment, the method of the invention may be used to provide an expressed fragment, or usually rather a collection of such expressed fragments, that is associated with a difference in a property relating disease resistance between the respective plant materials. Generally, such a collection of expressed fragments will contain at least 5 such fragments, e.g. between 6 and 1000 such fragments, preferably between 50 and 350 such fragments. In particular, the method of the invention may be used to provide a “bank” or “library” of such expressed fragments, e.g. a collection of two or more such fragments that comprises essentially all such fragments obtained from the plant materials used (or at least from the first plant material and the second plant material), e.g. covering essentially all the differences in the transcript profiles generated using the method of the invention for the respective plant materials. Generally, such a library or bank of expressed fragments will contain at least 5 such fragments, e.g. between 6 and 1000 such fragments, preferably between 50 and 350 such fragments. The expressed fragments obtained using the method of the invention, as well as any collection, bank or library of such fragments, form a further aspect of the invention.

The expressed fragments are preferably in the form of nucleotide sequences or nucleic acid fragments, in preferably in the form of amplified restriction fragments, and more preferably in the form of cDNA AFLP® fragments. These sequences and/or fragments, as well as collections, banks or libraries thereof (as defined above), form further aspects of the invention. Optionally, the nucleotide sequences/fragments may also be provided as one or more (isolated) nucleic acids, and these isolated nucleic acids, as well as collections, banks or libraries thereof (as defined above), form further aspects of the invention.

These expressed fragments, the manner in which they were obtained, and the manner in which the transcription profiles were generated using cDNA AFLP®, are further described in the Examples hereinbelow. The transcript profiles thus obtained were compared, which provided a collection of expressed fragments. Essentially, this collection constitutes a bank or library of all the expressed fragments that are representative for the differences in expression between the various plant materials as mentioned above, e.g. associated with the differences in a property relating to disease resistance between these plant materials as detected using cDNA AFLP® carried out as outlined above. In particular, expressed fragments in the collection correspond to genes the expression of which is regulated, directly or indirectly by the R— genes. This collection of expressed fragments, as well as the individual expressed fragments thereof, form further aspects of the invention.

B. Uses of the Differentially Expressed Fragments.

In a further aspect the invention relates to the various uses of the differentially expressed fragments of the invention.

A particularly preferred use of the expressed fragments of the invention concerns their use in the isolation of plant nucleotide sequences corresponding to the differentially expressed fragments. Useful plant nucleotide sequences to be isolated by means of the expressed fragments of the invention include e.g. full-length or partial genes corresponding to the expressed fragments, full-length cDNAs corresponding to those genes, regulatory sequences associated with those genes, flanking sequences upstream or downstream from those genes or even neighbouring genes. Most preferred nucleotide sequences are, however, genomic or cDNA sequences comprising a full-length coding sequence corresponding to the expressed fragment. The nucleotide sequences to be isolated by means of the expressed fragments of the invention are used to alter disease resistance in a plant or to confer upon a plant one or more favourable properties relating to disease resistance as described herein below.

There are a number of means available and known per se to the skilled person for isolation of the nucleotide sequences corresponding to the expressed fragments of the invention. The expressed fragments themselves may e.g. be used as hybridisation probes in screening genomic- or cDNA-libraries to isolate the corresponding clones. Such use does not even require knowledge of the sequence of the expressed fragment. Alternatively, the sequence of the expressed fragment may be determined and subsequently used to design oligonucleotides to be used as primers in variety of possible amplification reactions aimed at specifically amplifying genomic or cDNA fragments which may then be assembled into their full-length counterparts.

In another preferred aspect the invention relates to the use of the expressed fragments to down regulate expression of the corresponding genes by means of expression of (part of) the sequence of the expressed fragment as an antisense RNA molecule, being capable of interfering with translation of the corresponding mRNA to which the antisense expressed fragment is complementary. For this purpose the (part of) the sequence of the expressed fragment is cloned in antisense orientation in an expression vector. The antisense expression vector is constructed and subsequently introduced and expressed in a desired plant host cell or organism as described herein below.

In a further embodiment the expressed fragments of the invention are used in determining whether a plant or plant material has the (capacity to express) a desired property relating to disease resistance. E.g. the expressed fragments may be used in analysing the genome of a plant for the presence of genes corresponding to such a desired property relating to disease resistance; and/or for analysing a plant or plant material for the expression of transcripts associated with such desired property. The expressed fragments of the invention may also be used in the identification of loci—including e.g. QTL's—involved in a desired property relating to disease resistance, and/or to identify alleles of involved in such a desired property. When the expressed fragments of the invention are used in genome or expression analysis, a preferred aspect of the invention concerns a solid support containing one or more immobilised expressed fragments of the invention.

Such solid supports or carriers containing one or more immobilised nucleic acid fragments are known as (micro)arrays or DNA-chips for analysing nucleic acid sequences or mixtures thereof (see e.g. WO 97/27317, WO 97/22720, WO 97/43450, EP 0 799 897, EP 0 785 280, WO 97/31256, WO 97/27317 and WO 98/08083). Such an array will generally comprise at least 10, 20, 50, 100, 200, 420 or 1000 different expressed fragments of the invention. For a “high-density array” or “micro-array”, the total number of different expressed fragments can be in the range of 1000-100.000 per cm² of surface area. The fragments are preferably bound to the carrier in such a way that each fragment is attached to a specific and distinct part of the carrier, so as to form an independently detectable area on the carrier, such as a spot or band. This makes it possible to “read” the array by scanning (i.e. visually or otherwise) the areas to which each fragment is attached. Preferably, the independently detectable area's containing the individual fragments are organised on the carrier in a predetermined, and preferably regularly distributed pattern, i.e. in one or more lines, columns, rows, squares, rectangles, etc, preferably in an “addressable” form, this also includes beads as the carrier. This further facilitates analysis of the array. The density of the different fragments will generally be in the range of 1-100,000 different fragments/cm², usually 5-50,000 fragments/cm², generally between 10^{-10,000} fragments/cm². The solid support (i.e. carrier) for the array may be any solid material to which nucleic acid sequences can be attached such as e.g. plastics, resins, polysaccharides, silica or silica-based materials, functionalised glass, modified silicon, carbon, metals, inorganic glasses, membranes, nylon, natural fibres such as silk, wool and cotton, and polymer materials such as polystyrene, polyethylene glycol terephthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate and polymethylpentene. Further suitable support materials are mentioned for instance mentioned in U.S. Pat. No. 5,427,779, WO 97/22720, WO 97/43450, WO 97/31256, WO 97/27317 and EP 0 799 897. Preferably, the carrier will have an essentially flat, rectangular shape, with the fragments bound to one surface thereof. The size of the array, as well as of the individual areas corresponding to each of the different fragment may vary, depending upon the total amount of fragments, as well as the intended method for analysing the array. The fragments may be bound to the carrier in any manner known per se, and the specific technique used will mainly depend upon the carrier used. Binding will be at the 5′-end, or somewhere else on the fragments, as appropriate, but preferably not at the 3′-end, so as to allow for primer extension reactions. Preferably, the fragments are covalently linked to the array, i.e. by a suitable chemical technique. Suitable methods for attaching the fragments to the carrier will be clear to the skilled person. In general, any method for attaching a nucleic acid to a solid support can be used, including the methods described in U.S. Pat. No. 5,427,779; U.S. Pat. No. 4,973,493; U.S. Pat. No. 4,979,959; U.S. Pat. No. 5,002,582; U.S. Pat. No. 5,217,492; U.S. Pat. No. 5,525,041; U.S. Pat. No. 5,263,992; WO 97/46313 and WO 97/22720, as well as the references cited therein.

Further applications of the expressed fragments according to the invention will be clear to the skilled person, e.g. based upon the known uses of known/comparable expressed fragments.

C. Nucleic Acid Sequences, Polypeptides, DNA Constructs and Transgenic Plants.

One aspect of the invention relates to a nucleic acid molecule, preferably in isolated form that comprises a nucleotide sequence as defined in any of SEQ ID NO 1 to SEQ ID NO 420. In another aspect, the invention relates to a nucleic acid molecule, preferably in isolated form, that essentially consists of a nucleotide sequences as defined in any of SEQ ID NO 1 to SEQ ID NO 420. The invention further relates to mutants, variants, homologues, analogues, alleles, parts and/or fragments of the nucleotide sequences as defined in any of SEQ ID NO 1 to SEQ ID NO 420; and to nucleic acid molecules, preferably in isolated form, that comprise such mutants, variants, homologues, analogues, alleles, parts and/or fragments. Such mutants, variants, homologues, analogues, alleles, parts and/or fragments may differ from or may be derived from one of the nucleotide sequences of SEQ ID NO 1 to SEQ ID NO 420 by addition, substitution, insertion and/or deletion (herein collectively referred to as “nucleotide alterations”) of one or more bases/nucleotides at one or more positions. The mutant, variant, homologue analogue, allele, part and/or fragment of the nucleic acid molecules shall collectively be referred to as variants, variant nucleotide sequences or variant nucleic acid molecules. Preferably, such variants will have a degree of sequence identity of at least 40%, 50%, 60%, 70%, 80%, 90%, or more than 95%, 97%, 98% or 99% with one of the nucleotide sequences of SEQ ID NO 1 to SEQ ID NO 420.

For this purpose, the percentage of “sequence identity” between a given nucleotide sequence and one of the nucleotide sequences of SEQ ID NO 1 to SEQ ID NO 420 is calculated using a known computer algorithm for sequence alignment such as BLAST, PC-gene or Pedant-Pro, which may be used at suitable and/or at standard settings. For instance, the degree of homology may be calculated using the Pedant-Pro program at (one or more of) the settings given in Table 1:

TABLE 1
BLAST settings.

| | | Max. number of | Max. number of | Number of |
|---|---|---|---|---|
| | | alignments | description lines | iterations |
| Method | E-value | shown | shown | used |
| BLASTPGP | 0.001 | 10 | 500 | 1 |
| BLASTX | 0.01 | 5 | 500 | 5 |
| BLASTN | 10 | 5 | 500 | 1 |

Other settings:

| | |
|---|---|
| Minimal ORF length in ESTs (nt) | 9 |
| Maximal number of BLOCKS alignments shown | 10 |
| Blimps (BLOCKS) score threshold | 1100 |
| Threshold for reporting coiled coil | 0.95 |
| Minimal percentage of low complexity sequence | 20 |
| to be reported, % | |
| Threshold for reporting non-globular regions by SEG | 0 |
| Threshold for reporting PFAM hits | 0.001 |

More specifically, in the Examples the settings given in Table 2 were used.

TABLE 2
BLAST settings used in the Experimental Part:

| | | Max. number of | Max. number of | |
|---|---|---|---|---|
| | | alignments | description lines | Number of |
| Method | E-value | shown | shown | iterations used |
| BLASTPGP against the | 0.001 | 10 | 500 | 1 |
| protein databank | | | | |
| All-against-one alignment via | 0.0001 | 100 | 500 | 1 |
| BLASTPGP | | | | |
| BLASTPGP against | 0.001 | 10 | 500 | 5 |
| functional categories | | | | |
| BLASTPGP against COGs | 0.01 | 50 | 500 | 1 |
| BLASTPGP threshold for | 1e−05 | Not applicable | Not applicable | Not applicable |
| extracting PIR keywords, | | | | |
| superfamily information, and | | | | |
| EC numbers | | | | |
| BLASTX against the protein | 0.01 | 5 | 500 | 5 |
| databank | | | | |
| BLASTN against EMBL | 10 | 5 | 500 | 1 |
| BLASTN against EMBL- | 0.5 | 5 | 500 | 1 |
| EST | | | | |
| BLASTPGP against known | 0.001 | 50 | 500 | 1 |
| 3D structures | | | | |
| BLASTPGP against SCOP | 0.001 | 500 | 500 | 5 |
| domains | | | | |

Other settings:

| | |
|---|---|
| Minimal ORF length in ESTs (nt) | 9 |
| Maximal number of BLOCKS alignments shown | 10 |
| Blimps (BLOCKS) score threshold | 1100 |
| Threshold for reporting coiled coil | 0.95 |
| Minimal percentage of low complexity sequence to be reported, % | 20 |
| Threshold for reporting non-globular regions by SEG | 0 |
| Threshold for reporting PFAM hits | 0.001 |

Any part or fragment of a nucleotide sequence of SEQ ID NO 1 to SEQ ID NO 420 will preferably contain at least 30%, 40%, 50%, 60%, 70%, 80%, 90%, or more than 95% of the total number of nucleotides of the full nucleotide sequence of the pertinent SEQ ID. Two or more such parts or fragments of a nucleotide sequence of SEQ ID NO 1 to SEQ ID NO 420 may be joined or otherwise combined to provide a nucleotide sequence useful in the invention, in which case the total number of bases/nucleotides present in the combined parts of fragments should again preferably be within the percentages indicated above. Also, any such parts or fragments may contain further nucleotide alterations as defined above.

A variant nucleotide sequence of SEQ ID NO 1 to SEQ ID NO 420 may also be defined by its capability to hybridise with any of the nucleotide sequences of SEQ ID NO 1 to SEQ ID NO 420 under moderate, or preferably under stringent hybridisation conditions. Stringent hybridisation conditions are herein defined as conditions that allow a nucleic acid sequences of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity. Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

A particularly preferred nucleic acid molecule according to the invention comprises a coding sequence and a nucleotide sequence of SEQ ID NO 1 to SEQ ID NO 420 or a variant thereof. SEQ ID NO 1 to SEQ ID NO 420 comprise differentially expressed fragments and are thus derived from actively expressed transcripts that contain sequences coding for polypeptides. The nucleic acid molecule comprising a coding sequence and a nucleotide sequences of SEQ ID NO 1 to SEQ ID NO 420, preferably comprises a functional coding sequence, more preferably a full-length coding sequence. The nucleotide sequences of SEQ ID NO 1 to SEQ ID NO 420 will usually be present within the coding sequence, although the skilled person will appreciate that in some instances part or all of the nucleotide sequences of SEQ ID NO 1 to SEQ ID NO 420 may correspond to non-translated sequences in the transcript from which the nucleotide sequences of SEQ ID NO 1 to SEQ ID NO 420 is derived. In such instances the nucleotide sequences of SEQ ID NO 1 to SEQ ID NO 420 may be adjacent to or partly overlap with the coding sequence in the nucleic acid molecule. Thus, the invention relates to a nucleic acid molecule comprising a functional coding sequence for a plant polypeptide, wherein the coding sequence is part of a plant transcript that comprises a nucleotide sequence as defined in one of SEQ ID NO 1 to SEQ ID NO 420 is. The coding sequences in the nucleic acid molecules of the invention and variants thereof encode polypeptides that are further defined hereinbelow.

A nucleotide sequence comprising a sequence of SEQ ID NO 1 to SEQ ID NO 420 and the variants thereof is preferably such that the expression thereof in a suitable cell or organism, i.e. a host cell or host organism, leads to a “significant biological change” in the host cell or host organism. A “significant biological change” is understood to mean a detectable or measurable change in a biological property or activity of the host cell or host organism, as may be determined using one or more suitable detection methods known per se, such as one or more biological assays for the one or more biological properties or activities. In particular, a significant biological change may mean that the host cell or host organism, when maintained under conditions conducive to the expression of the nucleotide sequence or variant thereof, will show at least one of the biological properties and/or activities that are natively associated with the nucleotide sequences comprising a sequence of SEQ ID NO 1 to SEQ ID NO 420, and in particular one or more of the desired properties relating to disease resistance.

In particular, a variant of a nucleotide sequence comprising a sequence of SEQ ID NO 1 to SEQ ID NO 420 is such that, upon expression the nucleotide sequence and or the amino acid sequence encoded thereby, a biological property or activity is provided to the host cell or host organism in an amount of at least 30%, preferably at least 40%, more preferably at least 50%, even more preferably at least 75% and up to 100% or more, of the amount that is provided by a nucleotide sequences comprising a sequence of SEQ ID NO 1 to SEQ ID NO 420 in (essentially) the same host organism and under (essentially) the same conditions, as measured by a suitable assay for the a biological property or activity (in which it is to be understood that by “providing the biological property” also a reduction of one or more undesired properties may be meant, in which case the percentages mentioned above refer to the amount in which the undesired property is reduced compared to the reduction provided by one of the nucleotide sequences comprising a sequence of SEQ ID NO 1 to SEQ ID NO 420).

The nucleotide sequences comprising a sequence of SEQ ID NO 1 to SEQ ID NO 420 or variants thereof preferably encode an amino acid sequence of a polypeptide, such as a protein or an enzyme. It may also encode an RNA sequence that can influence one or more desired properties relating to disease resistance in or of the host. As will be clear to the skilled person, because of the degeneracy of the genetic code, a variant of a nucleotide sequence comprising a sequence of SEQ ID NO 1 to SEQ ID NO 420 may encode the same amino acid sequence as encoded by the nucleotide sequence comprising a sequence of SEQ ID NO 1 to SEQ ID NO 420. Such isocoding variant nucleotide sequences are included in the invention.

However, variants of the nucleotide sequences comprising a sequence of SEQ ID NO 1 to SEQ ID NO 420, may also encode a mutant, variant, homologue, analogue, allele, part and/or fragment of an amino acid sequence that is encoded by one of the nucleotide sequences comprising a sequence of SEQ ID NO 1 to SEQ ID NO 420. Again, such mutants, variants, homologues, analogues, alleles, parts and/or fragments of the amino acid sequences or polypeptides shall collectively be referred to as variants, variant amino acid sequences or variant polypeptides, proteins or enzymes. Such variant amino acid sequence may differ from the native amino acid sequence by addition, substitution, insertion and/or deletion of one or more amino acid residues at one or more positions, herein collectively referred to as “amino acid alterations”. Preferably, the variant amino acid sequences encoded by the nucleotide sequences of the invention will have a degree of amino acid sequence identity of at least 40%, 50%, 60%, 70%, 80%, 90%, or more than 95%, 97%, 98% or 99% with one of the an amino acid sequence encoded by a nucleotide sequence comprising a sequences of SEQ ID NO 1 to SEQ ID NO 420.

The degree of amino acid identity may be calculated using a known computer algorithm for sequence alignment such as BLAST, PC-gene or Pedant-Pro. E.g., the degree of amino acid homology may be calculated using the Pedant-Pro program at the settings given in Table 3.

TABLE 3
BLAST settings.

| | | Max. number | Max. number | Number of |
|---|---|---|---|---|
| | | of alignments | of description | iterations |
| Method | E-value | shown | lines shown | used |
| BLASTPGP | 0.001 | 10 | 500 | 1 |
| BLASTX | 0.01 | 5 | 500 | 5 |
| BLASTN | 10 | 5 | 500 | 1 |

Other settings

| | |
|---|---|
| Minimal ORF length in ESTs (nt) | 9 |
| Maximal number of BLOCKS alignments shown | 10 |
| Blimps (BLOCKS) score threshold | 1100 |
| Threshold for reporting coiled coil | 0.95 |
| Minimal percentage of low complexity sequence | 20 |
| to be reported, % | |
| Threshold for reporting non-globular regions by SEG | 0 |
| Threshold for reporting PFAM hits | 0.001 |

In particular, in the Examples the settings given in Table 4 were used.

TABLE 4
BLAST settings.

| | | Max. number | Max. number | Number of |
|---|---|---|---|---|
| | | of alignments | of description | iterations |
| Method | E-value | shown | lines shown | used |
| BLASTPGP against the protein databank | 0.001 | 10 | 500 | 1 |
| All-against-one alignment via | 0.0001 | 100 | 500 | 1 |
| BLASTPGP | | | | |
| BLASTPGP against functional categories | 0.001 | 10 | 500 | 5 |
| BLASTPGP against COGs | 0.01 | 50 | 500 | 1 |
| BLASTPGP threshold for extracting PIR | 1e−05 | Not | Not | Not |
| keywords, superfamily information, and | | applicable | applicable | applicable |
| EC numbers | | | | |
| BLASTX against the protein databank | 0.01 | 5 | 500 | 5 |
| BLASTN against EMBL | 10 | 5 | 500 | 1 |
| BLASTN against EMBL-EST | 0.5 | 5 | 500 | 1 |
| BLASTPGP against known 3D structures | 0.001 | 50 | 500 | 1 |
| BLASTPGP against SCOP domains | 0.001 | 500 | 500 | 5 |

Other settings

| | |
|---|---|
| Minimal ORF length in ESTs (nt) | 9 |
| Maximal number of BLOCKS alignments shown | 10 |
| Blimps (BLOCKS) score threshold | 1100 |
| Threshold for reporting coiled coil | 0.95 |
| Minimal percentage of low complexity sequence to be reported, % | 20 |
| Threshold for reporting non-globular regions by SEG | 0 |
| Threshold for reporting PFAM hits | 0.001 |

Optionally, in determining the degree of amino acid identity or homology, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

In the present invention, the nucleotide sequences comprising a sequence of SEQ ID NO 1 to SEQ ID NO 420 are preferably derived from varieties or lines of tomato, i.e. Lycopersicon esculentum, including Lycopersicon esculentum varieties like MoneyMaker or other Lycopersicon esculentum lines. However, it is envisaged that on the basis of the disclosure herein, the skilled person will be able to obtain nucleotide sequences homologous and preferably corresponding to nucleotide sequences comprising a sequence of SEQ ID NO 1 to SEQ ID NO 420 from other biological sources. Preferably such homologous sequences are obtained from plants, more preferably from dicotylous plants. Such homologous nucleotide or amino acid sequences will be referred to herein as “natural homologues” and are included within the scope of the present invention, as variants of such natural homologues. Preferably, any such natural homologue will have a degree of sequence identity of at least 30%, 40%, 50%, 60%, 70%, 80%, 90%, or more than 95%, 97%, 98% or 99% with of the nucleotide sequences comprising a sequence of SEQ ID NO 1 to SEQ ID NO 420 or an amino acid sequence encoded thereby. The percentage of sequence identity is calculated as set out above. Natural homologues of the nucleotide sequences comprising a sequence of SEQ ID NO 1 to SEQ ID NO 420 or variants thereof may also be defined by their capability to hybridise with a nucleotide sequences of SEQ ID NO 1 to SEQ ID NO 420 under moderate or preferably stringent hybridisation conditions as defined above. It will be understood that a natural homologue of one of the nucleotide sequences comprising a sequence of SEQ ID NO 1 to SEQ ID NO 420 or a variant thereof is preferably such that, in the biological source from which it was obtained, it has a biological property or is capable of a biological activity that is essentially the same or at least comparable to the biological properties or activities natively provided by the nucleotide sequences comprising a sequence of SEQ ID NO 1 to SEQ ID NO 420. Moreover, a natural homologue according to the invention is preferably such that the expression thereof in a host cell or host organism leads to a significant biological change as defined above in the host cell or host organism, in particular a significant biological change with respect to one or more desired properties relating to disease resistance as outlined above.

Hereinbelow, all of the nucleotide sequences and nucleic acid molecules described above, i.e. any and all nucleotide sequences comprising a sequence of SEQ ID NO 1 to SEQ ID NO 420, any natural homologues thereof, as well as any variants thereof will collectively be referred to as “nucleotide sequences of the invention”. The term “nucleotide sequences of the invention” also includes any and all nucleic acid molecules comprising such nucleotides sequences in the form of a nucleic acid construct as defined hereinbelow. These nucleotide sequences of the invention may be in the form of a double stranded nucleic acid or in form of a single stranded nucleic acid, in which the latter also includes any nucleic acid complementary to a nucleotide sequence of the invention. As such, the nucleotide sequences/nucleic acids of the invention may be a DNA sequence and/or an RNA sequence, which is again preferably in isolated form and/or in the form of a nucleic acid construct as defined hereinbelow. They may e.g. be genomic DNA sequences that—besides the one or more ORFs and/or exon sequences—may contain one or more intron sequences, 5′-UTRs or 3′-UTRs. They may also be cDNA sequences and/or RNA sequences, including but not limited to mRNA sequences.

Also included in the nucleotides sequences of the invention is any nucleotide sequence that is functionally homologous to any of the nucleotide sequences of SEQ ID NO 1 to 420 By the term “functionally homologous” encompasses the following. A sequence (for instance a gene) is considered functionally homologous if that sequence (gene) is homologous to another sequence, hence at least one nucleotide is deleted, inserted, replaced such as inversed (in case of more than one nucleotide) or transversion (purine-pyrimidine or pyrimidine-purine substitution) or transition (purine-purine or pyrimidine-pyrimidine substitution) while the function of said sequence (gene) is substantially maintained. This may also apply to chemically modified sequences. When a sequence is functionally homologous, there may very well be a low percentage of homology, but the functionality of that sequence is substantially maintained.

Also hereinbelow, any and all amino acid sequences encoded by the nucleotide sequences of the invention—and in particular those which meet the definitions for such amino acid sequences set out above—will be referred to hereinbelow as “polypeptides of the invention”. These polypeptides thus include oligopeptides, enzymes and non-catalytic proteins. The term functionally homologous as hereinbefore defined also applies to the amino acid sequences of the present invention. According to the invention, upon expression in a host cell or host organism, any polypeptide of the invention may also be subjected to one or more post-translational modifications, e.g. as may occur in the host cell or host organism, and any and all derivatives obtained as a result of such post-translational modification(s) are also included within the term “polypeptide(s) of the invention” as used herein. It will be clear to the skilled person that one or more of these post-translational modifications may increase, or may even be required for, one or more of the intended or desired biological properties or activities of the polypeptide. Instead of a polypeptide, a nucleotide sequence of the invention may also encode an RNA sequence, such as an RNA sequence having (up- or down-) regulatory activity with respect to one or more biological processes or pathways within the host cells, and in particular with respect to one or more of the properties relating to disease resistance. In such instances, the term “polypeptide of the invention” is to be read as “expression product of the invention”.

In yet another aspect, the invention also provides genes, e.g. full-length genes preferably including appropriate expression signals, containing one or more of the nucleotide sequences of the invention. More in particular, according to this aspect, the invention provides a nucleic acid molecule, preferably in isolated form, whereby the nucleic acid molecule comprises the following sequence elements:
a first nucleotide sequence encoding an expression product, preferably a polypeptide;
a second nucleotide sequence chosen from the group consisting of SEQ ID NO 1 to SEQ ID NO 420 or a variant thereof, whereby preferably at least part of the second nucleotide sequence is present within the first nucleotide sequence;
preferably, a translational start codon at the 5′ end of the first nucleotide sequence;
preferably, a translational stop codon at the 3′ end of the first nucleotide sequence;
preferably, one or more elements operably linked to the (5′-end of) first nucleotide sequence, which elements are capable of initiation and/or control of transcription of at least the first nucleotide sequence, such as a promoter, an enhancer, or an upstream activating sequence; and
preferably, a element operably linked to the (3′-end of) first nucleotide sequence, which element is capable of termination of transcription downstream of at least the first nucleotide sequence.

The nucleic acid molecule may be in any suitable form, and may for instance be in the form of single stranded or double stranded DNA or RNA (with dsDNA being preferred); and/or may be part of or in the form of a nucleic acid construct, e.g. as further described hereinbelow. Such nucleic acid may have any suitable size, for instance between 50 and 10000 nucleotides, in particular between 100 and 5000. The nucleic acid may optionally also be present in a host or host cell e.g. as defined below, including but not limited to bacterial cells (e.g. of Agrobacterium) or a plant or animal cell, and as such may be integrated in the genome of the host cell or may be maintained episomally in the host cell. It will be clear to the skilled person that nucleotide sequences comprising a full-length gene and containing a nucleotide sequence of the invention—optionally in the form of a nucleic acid molecule as described above—may be used in the same way, and for the same purposes, as the nucleotide sequences of the invention; and that for many applications, the use of such nucleic acid molecules may even be preferred. Thus, in the invention in its broadest sense, such nucleic acid molecules essentially comprising a full-length gene and comprising a nucleotide sequence of the invention are also comprised within the term “nucleotide sequence of the invention”. Also, the invention in its broadest sense comprises polypeptides as defined above encoded by the full-length genes of the invention. Furthermore, the invention in it broadest sense comprises variants and natural and functional homologues of such full-length genes, as well as variants and natural and functional homologues of such polypeptides.

The pathogen in terms of the present invention encompasses all pathogens relevant to the plants or crops as mentioned hereinbelow. Such pathogens can be a soil or a leaf pathogen and can be an obligate, biotroph or necrotroph pathogen, more in particular, the pathogen is selected from the group of bacteria, viruses, nematodes, aphids, fungi and/or pests. Preferably the pathogen is selected from Cladosporium fulvum, TMV, PVX, Pseudomonas syringae, CMV, CGMMV, TRV, Alternaria alternata, Odium lycopersici, Phytophtorea infestans, Botrytis cinerea, Fusarium oxysporum, and Fusarium race 2 and 3.

The nucleotide sequences of the invention are used to impart valuable properties on a host cell or host organism (as defined below), and in particular on a plant or plant cell. In particular, the nucleotide sequences of the invention may be used with advantage to provide a host cell or host organism, and in particular to confer upon a plant, one or more desired properties relating to the development of disease resistance of a plant against a plant pathogen. More in particular the nucleotide sequences of the invention may be used to provide a plant which is susceptible to a pathogen with an improved resistance against that pathogen. To achieve this the nucleotide sequences of the invention can be transformed into the genetic material of the plant by any method known in the art. Upon expression of the nucleotide sequence of the invention, the defence mechanism of the plant can be induced and the plant can express a systemic HR leading to resistance against the pathogen.

This proposed utility of the nucleotide sequences of the invention is based on observations, which are further described in the Examples. These observations are based on a model system. This model system is based on an extensive differential cDNA AFLP® analysis that was performed on Cf4 tomato, 10-90 minutes after the onset of a systemic HR due to specific recognition of the Avr4 elicitor of the biotrophic fungus Cladosporium fulvum, and non-responding control plants. The tomato (Lycopersicon esculentum) that formed part of the model system is a transgenic tomato that expresses both the Avr4 elicitor of C. fulvum and the matching Cf4 resistance gene. Seeds derived from a cross between a tomato carrying Cf4 and a plant expressing Avr4 germinate normally. However, generally within a few days the seedlings become necrotic and die.

By a specific temperature treatment, however, the HR induction and subsequent death of the plants can be suppressed, with the result that the plant develops normally. When the suppressive temperature is released (to a permissive temperature) the plant develop necrotic spots and the expression of many defence related genes is induced. Subsequently, these tomato plants develop a systemic HR within a very short time span. One of the many advantages of this system is that no wounding of the plant occurs. Wounding is known to induce the expression of many genes including defence genes. Furthermore as no pathogen is directly involved there is also a diminished risk of isolation of contaminating genes. This model system advantageously allows for the isolation of an virtually unlimited amount of plant material in which gene expression is synchronised. cDNA AFLP® is a technique that can advantageously be used to identify differentially expressed genes. By comparing the gene expression patterns between transgenic Cf::Avr plants which are either repressed or are not repressed in HR, the genes involved in the induction of plant defence are identified. Analysis of the (RNA) sequences isolated at different time points after the de-repression of HR results in the identification of a specific classes of genes related to the signal transduction pathway. The first genes that are induced are those that encode components of the signal transduction cascades and/or transcription factors. The products of these genes will subsequently activate the expression of the genes involved in the defence system. When a specific time frame is selected, for instance in a very early stage of the HR, it is possible to search for specific genes that play a role in that particular time frame, for instance for genes that exert their function in the initiation of plant defence responses.

Differentially expressed genes are sequenced and the sequences are compared to sequences present in the databases. To this end a variety of algorithms and computer programs is available to the skilled man such as Blast searches, Bestfit, FASTA, TFASTA, the algorithms as disclosed by Smith and Waterman Adv. Appl. Math. 2:482 (1981), Needleman and Wunsch J. Mol. Biol. 48:443 (1970), Pearson and Lipman Proc. Natl. Acad. Sci. (U.S.A.) 85:2444 (1988), and Pedant Pro (Biomax).

The expression patterns are examined individually, by northern blots or in the alternative simultaneously by spotting the AFLP® fragments on microarrays. These fragments can subsequently hybridised with cDNA isolated at different time points after the release of HR repression. Techniques such as silencing, for instance Virus Induced Gene Silencing (VIGS) provides for identification of relevant genes. Expression of homologous plant gene sequences in for instance potato Virus X (PVX) result in post-transcriptional gene silencing. The silenced plants are tested for their ability to induce HR when provoked with a suitable (race-specific) elicitor. The candidate genes are used in the production of transgenic plants, for instance tomato or Arabidopsis to study (over)expression and/or inducible expression.

Possible further applications or uses of the nucleotide sequences of the invention include e.g.:
utilisation as probe to detect homologous sequences;
utilisation in classification of species and sub-species and individuals;
use in heterologous production of polypeptides in for example micro-organisms;
use in or for overexpression in original source;
use in or for overexpression in a heterologous source or host;
use in or for silencing in original source;
use in or for silencing in a heterologous source or host;
use as marker(s) for indirect selection;
use on DNA arrays or chips.

The nucleotide sequences of the invention share as a common technical feature that they may be used to impart on a host cell or host organism one or more of the properties and/or activities mentioned above.

The nucleotide sequences of the invention may be isolated from natural source(s)—i.e. as indicated above—or from any other suitable source. Alternatively, the nucleotide sequences of the invention may be provided by recombinant techniques known per se, including but not limited to automated DNA synthesis; site-directed mutagenesis; combining two or more parts of one or more of the nucleotide sequences of the invention (e.g. using one or more restriction enzymes and/or ligases); introduction of mutations that lead to the expression of a truncated polypeptide; introduction of mutations that lead to an altered expression level and/or profile, and other modifications known to the skilled person per se. E.g. a specific technique involves the introduction of mutations by means of a PCR reaction using one or more “mismatched” primers and using one or more of the nucleotide sequences of the invention as a template. The amplified products/fragments thus obtained may then be ligated to form new variants of the nucleotide sequences of the invention. These and other suitable techniques are for instance described in Sambrook et al, “Molecular Cloning: A Laboratory Manual” (2nd.ed.), Vols. 1-3, Cold Spring Harbor Laboratory (1989) of F. Ausubel et al, eds., “Current protocols in molecular biology”, Green Publishing and Wiley Interscience, New York (1987) and Saiki et al., Science 239 (1988), 487-491 or PCR Protocols, 1990, Academic Press, San Diego, Calif., USA.

In a further aspect, the invention relates to a nucleic acid construct, the construct at least comprising a nucleotide sequence of the invention; and optionally further elements of nucleic acid constructs known per se.

Such a nucleic acid construct may be in the form of a DNA sequence or RNA sequence, and is preferably in the form of a double stranded DNA sequence. It may also be in the form of plasmid or vector. The nucleic acid construct is preferably in a form suitable for transforming the intended host cell or host organism. In particular, the nucleic acid construct may be in a form suitable for transforming a plant or plant cell. The nucleic acid construct may also be such that, upon transformation, it is stably maintained, replicated and/or inherited in the host cell or host organism. Alternatively, the nucleic acid construct may be such that—upon transformation—it allows for (at least) the nucleotide sequence of the invention to be integrated into the DNA—and preferably into the genomic DNA—of the host cell or host organism. The nucleic acid construct may also be such that it allows for the expression of the nucleotide sequence of the invention in the host cell or host organism. It is preferably also such that it allows for the nucleotide sequence of the invention to be inherited from one generation of the host organism to the next. For either or both of these purposes, upon transformation, the nucleic acid construct may first be integrated into the DNA of the host cell or host organism as described above.

The “optional further elements” that may be present in the nucleic acid constructs of the invention include, but are not limited to, one or more expression regulating sequences such as a promoter, leader sequences, terminators, enhancers, integration factors, selection markers and/or reporter genes, intron sequences and/or matrix attachment (MAR) sequences. Preferably, any such optional further elements are “operably linked” to the nucleotide sequence of the invention and/or to each other. by which is generally meant that they are in a functional relationship with each other. For instance, a promoter is operably linked to a coding sequence if the promoter is able to initiate or otherwise control/regulate the transcription and/or expression of a coding sequence, in which case the coding sequence should be understood as being “under the control of” the promoter. Generally, when two nucleotide sequences are operably linked, they will be in the same orientation and usually also in the same reading frame. They will usually also be essentially contiguous, although this may not be required.

Preferably, the optional further elements of the nucleic acid construct are such that they are capable of providing their intended biological function in the host cell or host organism. For instance, a promoter, enhancer and/or terminator should be “operable” in the host organism, by which is meant that, in the pertinent host cell or host organism, the promoter should be capable of initiating or otherwise controlling/regulating the transcription and/or the expression of a nucleotide sequence—e.g. a coding sequence—to which it is operably linked as defined above.

A promoter for use in the nucleic acid constructs of the invention may be a constitutive promoter or an inducible promoter as further mentioned below; and suitable non-limiting examples of such promoters for use in for instance plants, animals, as well as bacteria, yeast(cells) algae/fungi and/or viruses are for instance described in WO 95/07463, WO 96/23810, WO 95/07463, WO 95/21191, WO 97/11094, WO 97/42320, WO 98/06737 and WO 98/21355.

A selection marker for use in the nucleic acid constructs of the invention should be capable of distinguishing—e.g. allow the detection and/or selection of—host organisms or host cells that contain the nucleic acid construct. For instance, such a selection marker may be any gene that can be used to select—under suitable conditions such as the use of a suitable selection medium—plants, plant material and plant cells that contain—e.g. as the result of a successful transformation—the nucleic acid construct containing the marker. A particularly preferred selection marker is the npt-gene, which can be selected using kanamycin.

A leader sequence for use in the nucleic acid constructs of the invention should be such that—in the pertinent host cell or host organism—it may allow for the desired post-translational modifications in the host cell or host organism; may direct the transcribed mRNA to a desired part or organelle of the host cell; and/or it may allow for secretion of the polypeptide from the host cell. As such, it may be a pro-, pre-, or pre/pro-sequence operable is the host cell or host organism.

A reporter gene for use in the nucleic acid constructs of the invention should be such that, in the pertinent host cell or host organism, it allows for the expression of a nucleotide sequence present on the nucleic acid construct to be detected.

Some non-limiting examples of such further elements such as terminators, transcriptional and/or translational enhancers and/or integration factors for use in for instance plants, animals, as well as bacteria, yeast(cells), algae/fungi and/or viruses are for instance described in WO 95/07463, WO 96/23810, WO 95/07463, WO 95/21191, WO 97/11094, WO 97/42320, WO 98/06737 and WO 98/21355.

Also, in the invention, an nucleotide sequence of the invention may be operably linked to a other sequence that encodes a further amino acid sequence such as a protein or polypeptide, so as to provide—upon expression—a fusion of an polypeptide of the invention and the further amino acid sequence.

The construct of the invention can be provided in a manner known per se, which generally involves techniques such as restricting and linking nucleic acids/nucleic acid sequences, for which reference is made to the standard handbooks, such as Sambrook et al, “Molecular Cloning: A Laboratory Manual” (2nd.ed.), Vols. 1-3, Cold Spring Harbor Laboratory (1989) of F. Ausubel et al, eds., “Current protocols in molecular biology”, Green Publishing and Wiley Interscience, New York (1987).

When the nucleic acid construct is to be used for the transformation of plants or plant cells and/or for the expression of a nucleotide sequence of the invention in a plant or plant cell, the nucleic acid construct may e.g. contain, in addition to a nucleotide sequence of the invention, one or more of the following genetic elements:
(a) a promoter operable in a plant or plant cell, including but not limited to constitutive promoters such as the nos-promoter, de CaMV 35S promoter, de actin promoter en de mas (mannopinesynthase) promoter; inducible promoters such as the WIN (“wound inducible”) promoter and the HMGR (HydroxymethylglutarylCoA reductase) promoter; inducible promoters such as described in WO 97/41228, an ethanol inducible promoter (WO 99/29881) and the steroid inducible promoter (PNAS 88 23, 10421-25; 1991), copper ion inducible promoter (Transgenic Res. 5, 2, 105-113; 1996), Tet repressor regulated promoter (Mol Gen Genet. 227-2, 229-237; 1991); tissue- or organ-specific promoters such as the patatin promoter, the GBSS promoter and the plastocyanine promoter and those mentioned in WO 97/41228, the fruit specific promoter 2A11 (WO 8809334), E4 and E8 (WO 9201790) and the promoters described in WO 9727308, WO 9717452, WO 9831812; and/or promoters that are specific for a particular phase in the life cycle and/or development of a plant such as the SAG12 promoter (derived from the Arabidopsis “senescence associated gene”);
(b) a terminator operable in a plant or plant cell such as nos-3′ terminator, CaMV 3′ terminator or tml terminator;
(c) an enhancer operable in a plant or plant cell such as the CaMV 35S enhancer sequence;
(d) a reporter gene suitable for use in plant or plant cell such as the gfp-gene, gus gene or luc gene;
(e) a selection marker such as the npt gene, hpt gene or bar gene.

A nucleic acid construct for the transformation of plants may be in the form of a plasmid, cosmid or vector, including but not limited to a binary vectors or co-integration vectors, e.g. suitable for use in the transformation of a plant or plant cell using Agrobacterium; and Agrobacterium strains comprising a nucleic acid construct as described herein form a further aspect of the invention. Examples of suitable vectors systems for use with Agrobacterium are for instance binary vectors such as pBI121 and derivatives thereof; co-integration vectors such as pGV1500 and derivatives of pBR322.

In a further aspect, the invention relates to a host organism or host cell that contains and/or that is transformed with a one or more of the nucleotide sequences of the invention. The host organism or host cell may be (a cell of) an unicellular organism or (a cell of) a multicellular organism. Examples of unicellular organisms that can used as host organism/host cells in the invention include, but are not limited to micro-organisms such as viruses, bacteria, algae, fungi, yeast and/or protozoa's. Examples of multicellular organisms that can used as host organisms—and/or the cells of which can be used as host cells—in the invention include, but are not limited to higher organisms such as plants and animals, including but not limited to non-humans mammals.

Preferably, the host organism/host cell is a plant/plant cell. In particular, the host organism/host cell may be a (a cell of) a mono- or dicotylous plant, preferably (a cell of) an agronomically important plant or crop, such as e.g. watermelon, avocado, raspberry, pineapple, grape, apple, pear, orange, wheat, barley, rye, maize, tomato, pepper, lettuce, cucumber, rice, pulse, clover, citrus fruits, banana, grapes, cassava, pea, strawberry, cotton, chilli, brinjal, bhindi, sugarbeet, soybean, oil seed rape, sunflower, cauliflower, beans, peas, of which tomato, pepper and aubergine (egg plant) are particularly preferred. Other agronomically important species, varieties and/or lines of plants/crops will be clear to the skilled person. And may for instance be selected from species from the genera of Avena, Agrostis, Antirrhinum, Arabidopsis, Asparagus, Atropa, Brassica, Beta, Bromus, Browaalia, Capsicum, Ciahorum, Citrullus, Citrus, Composita, Cucumis, Cucurbita, Datura, Daucus, Digitalis, Festuca, Fragaria, Geranium, Glycine, Gramina, Helianthus, Heterocallis, Hordeum, Hyoscyamus, Juglans, Lactuca, Linum, Lolium, Lotus, Lycopersicon, Majorana, Manihot, Medicago, Nemesis, Nicotiana, Onobrychis, Oryza, Panieum, Pelargonium, Pennisetum, Petunia, Phaseolus, Pisum, Ranunculus, Raphanus, Rosa, Salpiglossis, Secale, Senecio, Sinapis, Solanum, Sorghum, Trifolium, Trigonella, Triticum, Vigna, Vita and Zea.

The plant or plant cell is preferably solanacea, such as exemplified by tomato, eggplant, pepper, tobacco, potato and the like, more preferably tomato.

The host cell may be present in a host organism in vivo. For instance, when the host cell is a cell of a multicellular (host) organism, the host cell may be present in the multicellular host organism and/or in any part, tissue or organ of the multicellular host organism; and any multicellular organism containing such a transformed host cell should be considered included within the term “multicellular host organism” as used herein. Similarly, when reference is made herein to a “multicellular host organism”, this also includes any part, tissue, organ or cell of such a multicellular host organism; and or any material derived from and/or for such a multicellular host organism.

For instance, when the multicellular host is a plant, the term “plant” also includes any part, tissue, organ or cell of such a plant, including but not limited to roots, stems, stalks, leaves, petals, fruits, seeds, tubers, meristems, sepals, and flowers. It also includes material of or for such a plant, such as (again) fruits or other materials of the plant that are intended to be harvested; material that can be regenerated into a (mature) plant, including but not limited protoplasts and/or callus tissue; or material that can be cultivated into a mature plant, such as seed, seedlings and/or (other) propagation material and/or cultivation material, e.g. as mentioned below.

The host cell may also be present in vitro, e.g. in a culture of such host cells. This may be a culture of the unicellular host cell(s), such as a culture of one of the micro-organisms mentioned above. It may also be a culture of host cells derived from a multicellular organism, such as an in vitro culture of plant or animal cells, including but not limited to a culture of cells or of a cell line derived from native plant cells or animal cells.

According to one embodiment, the host cell or host organism that contains and/or that has been transformed with the a nucleotide sequence of the invention is capable of expressing the nucleotide sequence. In particular, according to this embodiment, the nucleotide sequence of the invention is expressed in the host cell, in the host organism or in a part, tissue, organ or cell thereof. More preferably, the expression in such that is leads to a significant biological change (as defined above) in the host cell or host organism.

The host cell may also be a cell suitable for transforming another (second) host cell. In such a case, the nucleotide sequence of the invention is preferably in a form of a nucleic acid construct suitable for transforming the other (second) host cell, in which the nucleic acid construct is preferably also such that it can be (independently) maintained and/or replicated in the (first) host cell. E.g., the first host cell may be a virus or micro-organism suitable for transforming (cells of) a higher organism, such as an Agrobacterium strain suitable for transforming plants or plant cells.

The nucleotide sequence of the invention, as well as the one or more further optional elements of the nucleic acid construct may be homologous or heterologous to the host cell or host organism. Suitable sources and/or methods for obtaining the nucleotide sequences of the invention have been described hereinabove. The nucleotide sequences encoding the further elements of the constructs may have been isolated and/or derived from a naturally occurring source—for instance as cDNA—and/or from known available sources (such as available plasmids, etc.), and/or may have been provided synthetically using known DNA synthesis techniques.

The nucleic acid construct can be transformed into the host cell or host organism by any suitable transformation technique known per se. Suitable transformation systems and techniques for the transformation of plants, animals, as well as bacteria, yeast(cells), algae/fungi and/or viruses are for instance described in WO 95/07463, WO 96/23810, WO 95/07463, WO 95/21191, WO 97/11094, WO 97/42320, WO 98/06737 and WO 98/21355, as well as in Sambrook et al, “Molecular Cloning: A Laboratory Manual” (2nd.ed.), Vols. 1-3, Cold Spring Harbor Laboratory (1989) of F. Ausubel et al, eds., “Current protocols in molecular biology”, Green Publishing and Wiley Interscience, New York (1987). Some examples of techniques and vector systems for the transformation of plants or plant cells include transformation using Agrobacterium; transformation using “denuded” DNA, for instance using particle bombardment or transformation of protoplasts using electroporation or treatment with PEG; as well as the transformation techniques mentioned in WO 97/41228. Suitable vector systems for transformation using denuded DNA include but are not limited to E. coli-vectors with a high copy number such as pUC-vectors and pBluescript II (SK+) vectors. Suitable vector systems for use with Agrobacterium have been mentioned hereinabove.

As mentioned above, upon such transformation, the nucleic acid construct may be incorporated into the genomic DNA of the host (cell), or it may be maintained and inherited independent from the host genome, as an episomal genetic element in the host cell.

Upon transformation, the host organism or host cell may be kept under or exposed to conditions such that expression of the nucleotide sequence of the invention is obtained, e.g. in a cell, tissue, part or organ thereof. Such conditions may depend upon the host cell or host organism used and/or on the promoter that is used to regulate the expression of the nucleotide sequence of the invention. Such conditions may for instance include the presence of a suitable inducing conditions, factors and/or compounds. In case of plants, suitable conditions for expression may for instance also include growing and/or cultivating the transformed plant, e.g. until it is mature and/or carries fruits or seed. Suitable conditions may also include growing or keeping the plant under conditions where the expression of the nucleotide sequence of the invention may be induced by factors such as stress—e.g. lack of water, light or nutrients—and/or where the plant may be subject to attack or damage by disease and/or pathogens such as viruses, fungi, bacteria, insects, nematodes or other pests; and/or damage by herbivores.

In a further aspect the invention pertains to a method for inducing, or preferably regulating the induction of the HR response in a plant by means temperature control. The method preferably comprises the steps of (a) growing a plant expressing a matching pair of a plant pathogen derived avirulence gene and a plant resistance gene, at a temperature that suppresses induction of the hypersensitive response; and (b) reducing the temperature of the plant to a temperature that is permissive for the induction of the hypersensitive response.

The transgenic plant that may be applied in the method of the invention forms another aspect of the invention. The plant preferably comprising a matching pair of a plant pathogen derived avirulence gene and a plant resistance gene. The plant resistance gene may be an endogenous gene, but may also be an exogenously provided transgene. The pathogen derived avirulence gene usually is a transgene. The plant resistance gene and the avirulence gene are preferably present and/or expressed in each (somatic) cell of the plant and are preferably integrated in the genome of the plant's cells. The plant resistance gene and the avirulence gene are preferably driven by plant promoters, or at least promoters that are active in plant cells, e.g. promoters derived from plant viruses. The promoters are preferably active during (most of) the vegetative phase of the plant and preferably in all tissues of the plant. Alternatively, the promoter driving the expression of at least one of the plant resistance gene and the avirulence gene, is a regulated and/or tissue specific promoter such that the induction of the hypersensitive response may be regulated and/or confined to one or more specific tissues. The transgenic plant is preferably grown to at least 4, 5, 6, 7, 8, 9, 10 or 11 days post-emergence of the hypocotyls, alive and viable and preferably also free of necrosis, necrotic spots and/or a hypersensitive response. More preferably the plant is fullgrown, alive and viable and preferably also free of necrosis, necrotic spots and/or a hypersensitive response.

In the method of the invention, the transgenic plants are grown at a temperature that suppresses induction of the HR. A suppressive temperature will usually be higher than room temperature (i.e. 20° C.), or at least higher than the temperature at which the plants are conventionally grown. Preferably, a suppressive temperature is at least 5, 10, 15 or 20° C. above room temperature, or more preferably above the temperature at which the plants are conventionally grown. On the other hand the temperature at which the plants are grown only needs to be so high that the induction of the HR is suppressed and is preferably not chosen so high that the growth of the plants is significantly affected. A suitable suppressive temperature will depend on the plant species, variety or line and on the given matching pair of avirulence gene and resistance gene and may even depend on other conditions. A suitable suppressive temperature, however, can easily be determined by the skilled person. E.g. by allowing seeds for the transgenic plants to germinate at a range of temperatures to determine the lowest temperature at which the seeds germinate and grow out without developing necrotic spots. Usually the permissive temperature will be equal or close to room temperature (i.e. 20° C.), or equal or close to the temperature at which the plants are conventionally grown. By growing the transgenic plants under an regime comprising such an elevated temperature, the onset of the plant defence response reaction by the interaction of the (products of) the avirulence gene and the resistance gene is suppressed. When the temperature is subsequently lowered to the permissive temperature, the defence response is mounted. It is possible that the permissive temperature is below room temperature or below the temperature at which the plants are conventionally grown. Again, a suitable permissive temperature can easily be established by the skilled person without undue experimentation, simply by lowering the temperature, e.g. stepwise or with a gradient, and at regular intervals determining the onset of the defence response by methods known in the art and especially by the method described in this application. As an example, for transgenic tomato plants of the MoneyMaker variety that are transgenic for the Cf-4 resistance gene and for the C. fulvum Avr4 avirulence gene, 33° C. is a suitable suppressive temperature and 20° C. is a suitable permissive temperature.

When the method according to the invention is applied to a group of transgenic plants it is possible to simultaneously mount the defence response in a group of plants. In one embodiment, the invention according pertains to a method for the simultaneous induction of a HR in a group of at least two transgenic plants containing an avirulence gene and a resistance gene corresponding to said avirulence gene comprising a step wherein the plants are subjected to a temperature controlled step. The present invention also relates to the use of this method in the regulation of the, preferably simultaneous and/or systemic, induction of the HR.

### EXAMPLES

### Example 1

### Identification of Sequences Involved in the Hypersensitive Response

Disease resistance in plants to pathogens is often based on the presence of specific resistance (R) genes in the plant and avirulence (Avr) genes in the pathogen. When the R and Avr gene match, the plant induces a large array of defence responses. One of these is the HR in which cells around the infection site undergo programmed cell death. The HR, in combination with other defence responses, prevents further ingress of the pathogen.

Transgenic tomato lines have been constructed from a construct comprising the plant resistance gene Cf-4 and a plant expressing the Cladosporium fulvum avirulence gene Avr4 to result in transgenic plants that express both the Cladosporium fulvum avirulence gene (Avr4) and the matching plant resistance gene (Cf-4). The construction of these Cf-4/Avr4 transgenic tomato lines (as well as the construction of Cf-9/Avr9 transgenic tomato lines) is described by Cai et al. (2001) Mol. Plant Pathol. 2: 77-86. When grown under repressive conditions (high temperature: about 33° C.) these plants grow normal, but under permissive conditions (room temperature: about 20° C.) the plant defence responses are activated. This results in a systemic and synchronised induction of the HR and whole plant death in approximately 48 hours. Using cDNA AFLP®, with RNA isolated at different time-points after temperature shift, the (key)genes involved in the induction and regulation of these defence responses are identified.

RNA was isolated, at ten different time-points after the temperature shift, from Cf4-Avr4 plants and Cf0 control plants. This material was used for cDNA AFLP® analysis resulting in fingerprints with a consistent- and highly reproducible banding pattern. Test gels were run and analysed to determine the most informative time-points for performing the expression analysis with the material isolated and to determine whether the screen should be carried out with +2/+2 or with +2/+3 primers. Based on these results it was decided to use the time-points 0, 30, 60 and 90 minutes after the temperature shift for the complete analysis using the Taq/Mse enzyme combination and all 1024 +2/+3 primer combinations.

Adapters:
Taq-rare:
5′-CTCGTAGACTGCGTAC-3′
3′-TCTGACGCATGGC-5′
Mse:
5′-GACGATGAGTCCTGAG-3′
3′-TACTCAGGACTCAT-5′
Primers:
Taq rare
5′-GTAGACTGCGTACCGA-3′
Mse:
5′-GATGAGTCCTGAGTAA-3′

At the 3′ end of each primer the +2/+3 selective nucleotides are located.

Running and analysing the gels with all 1024 primer combinations resulted in the expression pattern of approximately 50 000 “Cf0 bands” that were compared to the “Cf4::Avr4” homologues. Bands that are differentially expressed were selected and the exact levels of expression were determined using the software Quantar-Pro (obtainable from Keygene NV, Wageningen, the Netherlands). Subsequently the difference in expression level per time-point between the Cf4::Avr4 and the Cf0 control plants was determined. Based on these data 442 fragments were selected that showed a difference in expression of a factor ≧3 at two or more time-points or of a factor between 2.5 and 3 at three or four time-points. Of these 442 differentials 328 are up-regulated in the Cf4::Avr4 plants and 114 are down regulated. For approximately 75% of these fragments the expression in the Cf4::Avr4 plants was already up- or down-regulated at timepoint t=0. Some of these differentials result from differences in genetic background (e.g. GusA, NptII, Avr4, Cf4) but it could also be an indication that the repression of plant defence signaling at higher temperature is not absolute and some defence-related signalling already takes place. All 442 differentials were isolated from gel, re-amplified and subcloned in the pCR2.1 vector using the TA cloning strategy. Reamplification primers were:

M00L:
5′-GACGATGAGTCCTGAGTAA-3′
TR00L:
5′-CTCGTAGACTGCGTACCGA-3′

After transformation four independent clones were picked and the insert sizes were validated on a sequence gel. In cases were less than four colonies were obtained after transformation, or when all four clones contained an insert of the wrong size, the fragments were re-isolated from gel. Finally 420 clones were obtained that contained an insert of the correct size. 16 fragments could not be cloned and are omitted from further analysis.

All 420 clones were sequenced and the obtained sequences trimmed by APES. This software tool, developed by the applicant, recognises and removes the AFLP® adapter sequences and determines the quality of the sequence. In order to determine the potential function of the differentials, most of the sequences have been characterised using PEDANT. This program performs blast searches against all known public databases and determines the sequence homology on protein and DNA level. Furthermore it determines the homology on the predicted 3D protein structure and recognisable protein domains. Based on these analyses the 420 sequence fragments can be divided into three groups.

The first group (Table 5) contains the fragments that can be matched to the “internal controls”. These “internal controls” result from known differences in the genetic background between Cf0 and Cf4::Avr4 such as the Cf-4 introgression fragment and the T-DNA (that contains Avr4 together with GusA and NptII (Kanamycin) as selectable markers). The difference in expression levels of these genes varies between the 4.5 and 150. This difference is at least partially due to the fact the genes on the T-DNA are under the control of the constitutive CaMV 35S promoter while the Cf4 gene is under the control of its own promoter. All expected “internal controls” have been retrieved. One of these genes is the Cf4A (Hcr9-4E) resistance gene of which the expression is very low. This gene is also a member of a very homologous multi-gene family and therefore, almost impossible to identify on a traditional northern blot. The fact that this gene fragment is retrieved shows that cDNA AFLP® is a very powerful technique to identify differentially expressed genes of which the expression is very low.

The second group (Table 6) contains all those sequence fragments that share homology or similarity with known sequences in publicly available databases. To determine the function of the genes belonging to the second group blast searches were performed. Homology searches on protein level show that approximately 40% of these sequences show significant homology to known plant sequences. A subset of the genes that have been identified so far are present in tomato cDNA libraries that were made from plants treated with biotic elicitors (underscored in the column marked ‘description’) or from developing tissues (ripening fruits, flowerbuds, roots and callus). This observation suggests that plant development and plant defence systems use, at least partially, the same signalling pathways. Based on the results obtained the genes are subdivided in 21 super-families (Table 7). These super-families are shown together with one or two references of the gene(s) with the highest homology. Most genes found have high homology to plant genes involved in plant defence. Besides many (receptor like) kinases, which are known to be involved in defence signaling also phospholipases are found that are involved in lipid-based signal transduction over the membranes. One of the first responses to elicitors is the oxidative burst and many genes are found that encode for peroxidases and cytochromes involved in the generation of these active oxygen species. This burst triggers expression of many late defence genes and accordingly a substantial number of genes coding for transcription factors is found. For the onset of the HR, which is a form of programmed cell death, it is known that many proteins are specifically degraded. The expression of many proteases is indeed up-regulated. Furthermore, in the first 90 minutes after the HR-induction some genes are already up-regulated that encode for proteins that play a role in later stages of defence. These proteins are for instance involved in phytoalexin production (like catechol oxidase) or known as PR proteins (chitinaseA).

### Example 2

### Virus Induced Gene Silencing

To functionally characterise the obtained nucleotide sequences are cloned in a virus which upon infection of the plant will result in virus induced gene silencing (VIGS). Not only the viral genes will be silenced but also the genes that are homologous to the nucleotides of the invention. Gene functionality is subsequently determined by analysing the silenced plants for the ability to mount a defence response upon elicitor treatment. Potato Virus X (PVX) is used as an expression vector and as a vector to induce gene silencing in Nicotiana Benthamania expressing Cf4 or Cf9. The differentials found are sub-cloned into the PVX vector. These clones are used for silencing on tobacco (N. Benthamania). As controls viruses containing part of the Cf-4 resistance gene (loss of HR) and the phytoene desaturase (PDS) gene (induces photobleaching) in silenced tissue, resulting in white leaves are included. After reaching satisfactory levels of silencing, the silenced plants are challenged with the AVR4 elicitor and scored for induction of HR. Those genes that appear essential for the HR are selected. Also the tobacco rattle virus (TRV)-based gene silencing system is used for the induction of silencing in both tobacco and tomato. Genes that are essential for HR on Cf4/t Cf-9 tobacco plants are analysed in the TRV system, together with other genes that were not silenced in tobacco in this system.

### Example 3

### Gene Expression Analysis in Other Systems Using cDNA AFLP®

RNA was isolated from control plants together with tomato plants that were inoculated with at least four different pathogens. Using cDNA AFLP® the expression of these genes is studied. Many of the fragments found in the screen are derived from the same gene or are derived from genes that are involved Cladosporium-tomato interactions. These cDNA AFLP® fragments are omitted. The expression analysis is only carried out on the other cDNA AFLP® fragments. The expression data provide criteria to select the novel genes that are early expressed and induced in various systems. These genes are used for the generation of broad and durable resistance and/or for the isolation of a pathogen inducible promoter.

Gene expression analysis experiments for the determination of the expression of the cDNA AFLP® identified genes in other systems.

| | | Resistant/ |
|---|---|---|
| Pathogen | Type of pathogen | susceptible lines |
| Cladosporium fulvum | Biotroph | R + S lines |
| Alternaria alternata | Necrotroph, AAL toxin | R + S lines |
| Oidium lycopersici | Obligate biotroph | S line |
| Phytophthora infestans | Semi biotroph | S line |
| Pseudomonas syringae | Bacteria | |
| Tobacco mosaic virus | Virus | |
| Tobacco rattle virus | Virus | |

### Example 4

### Functional Analysis of Candidate Disease Resistance Genes and Characterisation of Full Length Transcripts

Cloning of cDNA AFLP® fragments is carried out by cloning them into suitable vectors that facilitate handling and construction of other vectors and bacterial strains. Subsequently characterisation of full length transcripts is performed in order to obtain sequence information of the full length transcripts. In the absence of information on sequence homology in the database, the cDNA sequence is obtained by RACE. Southern hybridisation experiments confirms the single copy nature of the clones

After cloning the full-length cDNAs they are inserted under the control of suitable promoters in transformations vectors. After transfer of the vector to Agrobacterium the strains are used for the generation of transgenic tomato pants. From each construct a number of independent diploid transformants are generated and after ploidy analysis, copy number counts and verifying the intact integration, the fully grown plants are allowed to set seed ad the next generation is analysed to reveal the exact function of the gene—product in plant defence. The plants are further analysed for morphological differences and the timing of the induction of a defence response for instance by using northern blot hybridisation with known PR genes, microarray gene expression analysis of cDNA AFLP® in either the absence or the presence of various pathogens.

TABLE 5
cDNA-AFLP fragments that can be assigned to known differences in genetic
background.

| SEQ ID | | | | | |
|---|---|---|---|---|---|
| NO | Fragment | Accesion | Protein | Homology | Up-regulation |
| 28 | Km3f_tr11-76r | TREMBL: AG146_1 | Neomycin | 8e−22 | 10x |
| | | | phosphotransferase | | |
| | | | resistance protein; | | |
| | | | Kanamycin resistance | | |
| 60 | Km3f_tr11-53r | PIR: T07015 | Cf-4A protein - tomato | 1e−52 | 4.5x |
| 166 | km3f_t23-G05r | | HygromycinB | 2e−38 | 14x |
| | | | phosphotransferase | | |
| 362 | Km3f_tr15-32r | PIR: S41047 | AVR4 protein - fungus | 2e−10 | 28x |
| | | | (Cladosporium fulvum) | | |

TABLE 6
Sequence fragments with similarity to known sequences

| SEQ | | | | | |
|---|---|---|---|---|---|
| ID NO. | Contig | Description | Best BLAST hit | Hit ID | e-val |
| 1 | km3f_reverse_tr11- | BLASTX | cathepsin D inhibitor precursor - potato | PIR: S52656 | 5e−21 |
| | 10r | orf | | | |
| 2 | km3f_reverse_tr11- | BLASTX | ferredoxin 2[4Fe-4S] frxB - common | PIR: FENTB | 2e−48 |
| | 13r | orf | tobacco chloroplast | | |
| 3 | km3f_reverse_tr11- | Longest orf | product: “KE03 protein”; Homo sapiens | TREMBL: AF064604_1 | 0.36 |
| | 14r | | KE03 protein mRNA, partial cds. | | |
| 4 | km3f_reverse_tr11- | BLASTX | gene: “F24P17.1”; product: “putative | TREMBL: AC011623_1 | 1e−33 |
| | 19r | orf | pyruvate dehydrogenase kinase, 5′ | | |
| | | | partial”; Arabidopsis thaliana | | |
| | | | chromosome III BAC F24P17 genomic | | |
| | | | sequence, complete sequence. | | |
| 5 | km3f_reverse_tr11-1r | BLASTX | gene: “P0009G03.30”; product: “putative | TREMBL: AP002522_30 | 7e−21 |
| | | orf | protein kinase Xa21”; Oryza sativa | | |
| | | | genomic DNA, chromosome 1, PAC | | |
| | | | clone: P0009G03. | | |
| 6 | km3f_reverse_tr11- | BLASTX | gene: “T4K22.4”; product: “unknown | TREMBL: AC025295_7 | 2e−05 |
| | 25r | orf | protein”; Arabidopsis thaliana | | |
| | | | chromosome 1 BAC T4K22 genomic | | |
| | | | sequence, complete sequence. | | |
| 8 | km3f_reverse_tr11- | BLASTX | leucyl aminopeptidase (EC 3.4.11.1) | PIR: T07850 | 2e−16 |
| | 28r | orf | (clone pBlap2) precursor, wound- | | |
| | | | induced - tomato (fragment) | | |
| 13 | km3f_reverse_tr11- | BLASTX | hypothetical protein T20K18.180 - | PIR: T06641 | 5e−27 |
| | 45r | orf | Arabidopsis thaliana | | |
| 15 | km3f_reverse_tr11- | Longest orf | neoxanthin cleavage enzyme nc1 - | PIR: T49193 | 0.13 |
| | 47r | | Arabidopsis thaliana | | |
| 17 | km3f_reverse_tr11- | BLASTX | peroxidase (EC 1.11.1.7) - common | PIR: T03686 | 8e−06 |
| | 59r | orf | tobacco | | |
| 18 | km3f_reverse_tr11-5r | BLASTX | carbonate dehydratase (EC 4.2.1.1) | PIR: T02936 | 6e−12 |
| | | orf | precursor, chloroplast - common tobacco | | |
| 19 | km3f_reverse_tr11- | BLASTX | hypothetical protein F23E12.210 - | PIR: T06134 | 4e−04 |
| | 62r | orf | Arabidopsis thaliana | | |
| 22 | km3f_reverse_tr11- | BLASTX | product: “DNAJ-like protein”; | TREMBL: AB023028_1 | 3e−11 |
| | 65r | orf | Arabidopsis thaliana genomic DNA, | | |
| | | | chromosome 5, TAC clone: K20J1. | | |
| 23 | km3f_reverse_tr11- | BLASTX | Alfalfa histone H3 (H3-1.1) gene, | TREMBL: MSHISH3A_1 | 3e−06 |
| | 66r | orf | complete cds. | | |
| 28 | km3f_reverse_tr11- | BLASTX | product: “neomycin phosphotransferase”; | TREMBL: CV35137_1 | 2e−21 |
| | 76r | orf | Plasmid pBSL99 cloning vector, | | |
| | | | complete sequence. | | |
| 29 | km3f_reverse_tr11- | BLASTX | hypothetical protein F8F6.170 - | PIR: T48423 | 2e−04 |
| | 78r | orf | Arabidopsis thaliana | | |
| 30 | km3f_reverse_tr11- | Longest orf | product: “eukaryotic initiation factor 4, | TREMBL: AB013396_11 | 0.075 |
| | 81r | | eIF4-like protein”; Arabidopsis thaliana | | |
| | | | genomic DNA, chromosome 5, P1 | | |
| | | | clone: MTI20. | | |
| 33 | km3f_reverse_tr11- | BLASTX | gene: “F15K9.23”; Arabidopsis thaliana | TREMBL: AC005278_23 | 4e−08 |
| | 85r | orf | chromosome 1 BAC F15K9 sequence, | | |
| | | | complete sequence. | | |
| 34 | km3f_reverse_tr11- | BLASTX | product: “serine/threonine-specific | TREMBL: AB016879_9 | 7e−21 |
| | 88r | orf | protein kinase-like protein”; Arabidopsis | | |
| | | | thaliana genomic DNA, chromosome 5, | | |
| | | | P1 clone: MRB17. | | |
| 35 | km3f_reverse_tr11- | BLASTX | peroxidase (BC 1.11.1.7) precursor - | PIR: S07407 | 1e−09 |
| | 90r | orf | potato (fragment) | | |
| 37 | km3f_reverse_tr11- | BLASTX | gene: “F25P22.13”; product: | TREMBL: AC012679_12 | 4e−30 |
| | 94r | orf | “hypothetical protein; 49134-52109”; | | |
| | | | Arabidopsis thaliana chromosome 1 | | |
| | | | BAC F25P22 genomic sequence, | | |
| | | | complete sequence. | | |
| 38 | km3f_reverse_tr11- | BLASTX | Arabidopsis thaliana genomic DNA, | TREMBL: AB006703_6 | 1e−04 |
| | 95r | orf | chromosome 5, P1 clone: MRH10. | | |
| 39 | km3f_reverse_tr11-9r | BLASTX | cathepsin D inhibitor precursor - potato | PIR: S52656 | 5e−21 |
| | | orf | | | |
| 83 | km3f_reverse_tr1114- | BLASTX | hypothetical protein F25G13.100 - | PIR: T10203 | 4e−09 |
| | 86r | orf | Arabidopsis thaliana | | |
| 84 | km3f_reverse_tr1114- | BLASTX | hypothetical protein F23E12.210 - | PIR: T06134 | 0.001 |
| | 89r | orf | Arabidopsis thaliana | | |
| 86 | km3f_reverse_tr1114- | BLASTX | leucyl aminopeptidase (EC 3.4.11.1) | PIR: T07850 | 2e−16 |
| | 95r | orf | (clone pBlap2) precursor, wound- | | |
| | | | induced - tomato (fragment) | | |
| 185 | km3f_t19-a02r | BLASTX | gene: “EEF13”; Solanum melongena | TREMBL: AB032753_1 | 3e−12 |
| | | orf | EEF13 mRNA, complete cds. | | |
| 186 | km3f_t19-a03r | BLASTX | gene: “FIN219”; product: “FIN219”; | TREMBL: AF279129_1 | 2e−07 |
| | | orf | Arabidopsis thaliana FIN219 (FIN219) | | |
| | | | gene, complete cds. | | |
| 188 | km3f_t19-a05r | BLASTX | disease resistance protein D - tomato | PIR: T17461 | 9e−05 |
| | | orf | | | |
| 190 | km3f_t19-a07r | BLASTX | gene: “CesA-1”; product: “cellulose | TREMBL: AF200525_1 | 0.002 |
| | | orf | synthase-1”; Zea mays cellulose | | |
| | | | synthase-1 (CesA-1) mRNA, complete | | |
| | | | cds. | | |
| 191 | km3f_t19-a09r | BLASTX | Arabidopsis thaliana genomic DNA, | TREMBL: AB012242_14 | 8e−14 |
| | | orf | chromosome 5, TAC clone: K24G6. | | |
| 192 | km3f_t19-a10r | BLASTX | gene: “F3F20.1”; Arabidopsis thaliana | TREMBL: AC007153_1 | 3e−31 |
| | | orf | chromosome I BAC F3F20 genomic | | |
| | | | sequence, complete sequence. | | |
| 193 | km3f_t19-a11r | BLASTX | hypothetical protein a - maize | PIR: T02916 | 1e−15 |
| | | orf | transposable element Ac | | |
| 194 | km3f_t19-a12r | BLASTX | gene: “T1N6.12”; Sequence of BAC | TREMBL: AC009273_12 | 3e−22 |
| | | orf | T1N6 from Arabidopsis thaliana | | |
| | | | chromosome 1, complete sequence. | | |
| 196 | km3f_t19-b02r | BLASTX | product: “osmotin”; N. tabacum mRNA | TREMBL: NTOSMOTIN_1 | 1e−06 |
| | | orf | for osmotin | | |
| 198 | km3f_t19-b04r | BLASTX | disease resistance protein - tomato | PIR: T17460 | 2e−73 |
| | | orf | | | |
| 200 | km3f_t19-b07r | BLASTX | leucyl aminopeptidase (EC 3.4.11.1) | PIR: S57811 | 7e−11 |
| | | orf | (clone TPP6) - tomato (fragment) | | |
| 201 | km3f_t19.b08r | BLASTX | leucyl aminopeptidase (EC 3.4.11.1) | PIR: S57811 | 7e−11 |
| | | orf | (clone TPP6) - tomato (fragment) | | |
| 205 | km3f_t19-b12r | BLASTX | product: “zhb0001.1”; Oryza sativa | TREMBL: OST17804_1 | 2e−08 |
| | | orf | indica(GLA4) genomic DNA, | | |
| | | | chromosome 4, BAC clone: t17804. | | |
| 206 | km3f_t19-c01r | BLASTX | gene: “cathDInh”; product: “Cathepsin D | TREMBL: LES295638_1 | 2e−44 |
| | | orf | Inhibitor”; Lycopersicon esculentum | | |
| | | | cathDInh gene for Cathepsin D Inhibitor | | |
| 207 | km3f_t19-c02r | BLASTX | IMPORTIN ALPHA SUBUNIT | SWISSPROT: IMA_LYCES | 4e−49 |
| | | orf | (KARYOPHERIN ALPHA SUBUNIT) | | |
| | | | (KAP ALPHA) (FRAGMENT). | | |
| 209 | km3f_t19-c04r | BLASTX | product: “protein phosphatase-2C PP2C- | TREMBL: AB026661_10 | 4e−07 |
| | | orf | like”; Arabidopsis thaliana genomic | | |
| | | | DNA, chromosome 5, BAC | | |
| | | | clone: T30G6. | | |
| 210 | km3f_t19-c05r | BLASTX | Arabidopsis thaliana genomic DNA, | TREMBL: AB012246_7 | 3e−22 |
| | | orf | chromosome 5, P1 clone: MRG7. | | |
| 212 | km3f_t19-c07r | BLASTX | gene: “At2g46680”; product: | TREMBL: AC005819_6 | 6e−08 |
| | | orf | “homeodomain transcription factor | | |
| | | | (ATHB-7)”; Arabidopsis thaliana | | |
| | | | chromosome II section 248 of 255 of the | | |
| | | | complete sequence. Sequence from | | |
| | | | clones F13A10, T3A4. | | |
| 213 | km3f_t19-c08r | BLASTX | naringenin-chalcone synthase (EC | PIR: SYPJCB | 6e−10 |
| | | orf | 2.3.1.74) B - garden petunia | | |
| 215 | km3f_t19-c10r | BLASTX | disease resistance E - tomato | PIR: T17462 | 8e−30 |
| | | orf | | | |
| 218 | km3f_t19-d01r | BLASTX | leucyl aminopeptidase (EC 3.4.11.1) | PIR: A48788 | 3e−10 |
| | | orf | DR57 - tomato | | |
| 220 | km3f_t19-d03r | BLASTX | vacuolar proton-ATPase chain E - potato | PIR: T07110 | 9e−07 |
| | | orf | | | |
| 222 | km3f_t19-d05r | BLASTX | hypothetical protein F20D22.10 - | PIR: T00960 | 5e−36 |
| | | orf | Arabidopsis thaliana | | |
| 223 | km3f_t19-d06r | BLASTX | CYTOCHROME P450 LXXVIIA1 (EC | SWISSPROT: C771_SOLME | 1e−05 |
| | | orf | 1.14.14.1) (P-450EG6) (FRAGMENT). | | |
| 224 | km3f_t19-d08r | BLASTX | chlorophyll a/b-binding protein (cab-36) - | PIR: S21827 | 5e−06 |
| | | orf | common tobacco | | |
| 226 | km3f_t19-d11r | BLASTX | gene: “AATL1”; product: “amino acid | TREMBL: AB030586_1 | 3e−21 |
| | | orf | transporter-like protein 1”Arabidopsis | | |
| | | | thaliana AATL1 gene for amino acid | | |
| | | | transporter-like protein 1, complete cds. | | |
| 228 | km3f_t19-e01r | BLASTX | heat shock protein 18p - common | PIR: T03958 | 2e−17 |
| | | orf | tobacco | | |
| 230 | km3f_t19-e03r | BLASTX | phosphopyruvate hydratase (EC 4.2.1.11) - | PIR: JQ1185 | 4e−29 |
| | | orf | tomato | | |
| 231 | km3f_t19-e04r | BLASTX | subtilisin-like proteinase (EC 3.4.21.—) - | PIR: T06577 | 6e−14 |
| | | orf | tomato | | |
| 232 | km3f_t19-e05r | BLASTX | leucyl aminopeptidase (EC 3.4.11.1) | PIR: T07047 | 9e−11 |
| | | orf | lap17.1a - tomato | | |
| 233 | km3f_t19-e06r | BLASTX | gene: “pgm I”; product: “cytosolic | TREMBL: STU240054_1 | 9e−07 |
| | | orf | phosphoglucomutase”; Solanum | | |
| | | | tuberosum mRNA for cytosolic | | |
| | | | phosphoglucomutase | | |
| 234 | km3f_t19-e07r | BLASTX | Arabidopsis thaliana genomic DNA, | TREMBL: AB017063_15 | 4e−28 |
| | | orf | chromosome 5, TAC clone: K3K7. | | |
| 236 | km3f_t19-e09r | BLASTX | gene: “F21F23.9”; product: “F21F23.9”; | TREMBL: AC027656_9 | 3e−10 |
| | | orf | Sequence of BAC F21F23 from | | |
| | | | Arabidopsis thaliana chromosome 1, | | |
| | | | complete sequence. | | |
| 237 | km3f_t19-e10r | BLASTX | product: “receptor-like protein kinase”; | TREMBL: AB016892_2 | 3e−20 |
| | | orf | Arabidopsis thaliana genomic DNA, | | |
| | | | chromosome 5, P1 clone: MXF12. | | |
| 238 | km3f_t19-e11r | BLASTX | retrovirus-related reverse transcriptase | PIR: S04273 | 3e−13 |
| | | orf | homolog - common tobacco | | |
| | | | retrotransposon copia-like | | |
| 239 | km3f_t19-e12r | BLASTX | gene: “At2g26980”; product: “putative | TREMBL: AC005623_4 | 9e−36 |
| | | orf | protein kinase”; Arabidopsis thaliana | | |
| | | | chromosome II section 151 of 255 of the | | |
| | | | complete sequence. Sequence from | | |
| | | | clones T20P8, F20F1. | | |
| 240 | km3f_t19-f01r | BLASTX | gene: “T32B20.f”; product: | TREMBL: AF262041_1 | 0.005 |
| | | orf | “Hypothetical protein T32B20.f”; | | |
| | | | Arabidopsis thaliana BAC T32B20. | | |
| 241 | km3f_t19-f02r | BLASTX | gene: “T13O15.1”; Arabidopsis thaliana | TREMBL: AC010870_1 | 3e−08 |
| | | orf | chromosome III BAC T13O15 genomic | | |
| | | | sequence, complete sequence. | | |
| 242 | km3f_t19-f03r | BLASTX | gene: “At2g48110”; Arabidopsis thaliana | TREMBL: AC006072_12 | 9e−18 |
| | | orf | chromosome II section 255 of 255 of the | | |
| | | | complete sequence. Sequence from | | |
| | | | clones T9J23, F11L15, pAtT51. | | |
| 245 | km3f_t19-f06r | Longest orf | hypothetical protein PH0220 - | PIR: B71245 | 0.16 |
| | | | Pyrococcus horikoshii | | |
| 247 | km3f_t19-f08r | Longest orf | product: “putative DMO orthologue”; | TREMBL: HSA290954_1 | 0.26 |
| | | | Homo sapiens partial mRNA for putative | | |
| | | | DMO orthologue | | |
| 249 | km3f_t19-f10r | BLASTX | gene: “At2g15790”; product: “putative | TREMBL: AC006438_4 | 3e−20 |
| | | orf | cyclophilin-type peptidyl-prolyl cis-trans | | |
| | | | isomerase”; Arabidopsis thaliana | | |
| | | | chromosome II section 92 of 255 of the | | |
| | | | complete sequence. Sequence from | | |
| | | | clones F19G14, F7H1. | | |
| 250 | km3f_t19-f11r | BLASTX | hypothetical protein F16A16.170 - | PIR: T04527 | 1e−04 |
| | | orf | Arabidopsis thaliana | | |
| 251 | km3f_t19-f12r | BLASTX | pathogenesis-related protein P6 precursor - | PIR: VCTO14 | 2e−36 |
| | | orf | tomato | | |
| 252 | km3f_t19-g01r | BLASTX | leucyl aminopeptidase (EC 3.4.11.1) | PIR: S57811 | 7e−11 |
| | | orf | (clone TPP6) - tomato (fragment) | | |
| 253 | km3f_t19-g02r | BLASTX | probable P-glycoprotein pgp1 - | PIR: T00558 | 4e−05 |
| | | orf | Arabidopsis thaliana | | |
| 256 | km3f_t19-g05r | BLASTX | pathogenesis-related protein P6 precursor - | PIR: VCTO14 | 3e−62 |
| | | orf | tomato | | |
| 257 | km3f_t19-g06r | BLASTX | serine/threonine protein kinase - | PIR: T50501 | 7e−11 |
| | | orf | Arabidopsis thaliana | | |
| 258 | km3f_t19-g07r | BLASTX | Arabidopsis thaliana genomic DNA, | TREMBL: AP002031_4 | 4e−13 |
| | | orf | chromosome 5, TAC clone: K3D20. | | |
| 259 | km3f_t19-g08r | BLASTX | nematodes resistance protein Mi-1.1 - | PIR: T06267 | 4e−09 |
| | | orf | tomato | | |
| 261 | km3f_t19-g10r | BLASTX | product: “copia-type pol polyprotein- | TREMBL: AP002459_3 | 2e−08 |
| | | orf | like”Arabidopsis thaliana genomic | | |
| | | | DNA, chromosome 3, BAC | | |
| | | | clone: T13B17. | | |
| 263 | km3f_t19-g12r | BLASTX | Synthetic E. coli ORF16/lacZ fusion | TREMBL: AGORFLAC_1 | 2e−04 |
| | | orf | protein, partial cds. | | |
| 264 | km3f_t19-h01r | BLASTX | product: “peptidylprolyl isomerase”; | TREMBL: AB015468_3 | 4e−57 |
| | | orf | Arabidopsis thaliana genomic DNA, | | |
| | | | chromosome 5, TAC clone: K15N18. | | |
| 265 | km3f_t19-h02r | BLASTX | nitrate reductase (NADH) (EC 1.6.6.1) - | PIR: RDTONH | 2e−26 |
| | | orf | tomato | | |
| 267 | km3f_t19-h04r | BLASTX | RIBULOSE BISPHOSPHATE | SWISSPROT: RBL_LYCES | 7e−21 |
| | | orf | CARBOXYLASE LARGE CHAIN | | |
| | | | PRECURSOR (EC 4.1.1.39). | | |
| 268 | km3f_t19-h05r | BLASTX | product: “receptor protein kinase”; | TREMBL: AB028616_8 | 0.032 |
| | | orf | Arabidopsis thaliana genomic DNA, | | |
| | | | chromosome 3, P1 clone: MMG15. | | |
| 271 | km3f_t19-h08r | BLASTX | ribulose-bisphosphate carboxylase (EC | PIR: RKTO3B | 4e−31 |
| | | orf | 4.1.1.39) small chain 3B precursor - | | |
| | | | tomato | | |
| 272 | km3f_t19-h09r | BLASTX | pollen-specific protein homolog - tomato | PIR: T07129 | 1e−08 |
| | | orf | (fragment) | | |
| 273 | km3f_t19-h10r | BLASTX | subtilisin-like proteinase (EC 3.4.21.—) 3 - | PIR: T07169 | 2e−22 |
| | | orf | tomato | | |
| 275 | km3f_t19-h12r | BLASTX | hypothetical protein F26K9.90 - | PIR: T48055 | 2e−26 |
| | | orf | Arabidopsis thaliana | | |
| 95 | km3f_t23-a05r | BLASTX | gene: “dof1”; product: “Dof zinc finger | TREMBL: STU242853_1 | 2e−15 |
| | | orf | protein”; Solanum tuberosum mRNA for | | |
| | | | Dof zinc finger protein (dof1 gene) | | |
| 97 | km3f_t23-a07r | BLASTX | class I patatin - potato | PIR: T07592 | 7e−09 |
| | | orf | | | |
| 98 | km3f_t23-a08r | BLASTX | product: “GUSA (N358Q); hexaHis | TREMBL: AF234302_1 | 3e−16 |
| | | orf | tagged”; Binary vector pCAMBIA-1381, | | |
| | | | complete sequence. | | |
| 99 | km3f_t23-a10r | BLASTX | gene: “WRKY1”; product: “WRKY | TREMBL: STU278507_1 | 9e−23 |
| | | orf | DNA binding protein”; Solanum | | |
| | | | tuberosum mRNA for WRKY DNA | | |
| | | | binding protein (WRXY1 gene) | | |
| 102 | km3f_t23-b01r | BLASTX | probable calcium binding protein - | PIR: T45708 | 1e−05 |
| | | orf | Arabidopsis thaliana | | |
| 104 | km3f_t23-b03r | BLASTX | cytochrome P450 76A2 - eggplant | PIR: S38534 | 1e−19 |
| | | orf | | | |
| 111 | km3f_t23-b10r | BLASTX | gene: “F17A9.21”; product: “putative | TREMBL: AC016827_20 | 8e−17 |
| | | orf | GTPase”; Arabidopsis thaliana | | |
| | | | chromosome III BAC F17A9 genomic | | |
| | | | sequence, complete sequence. | | |
| 112 | km3f_t23-b11r | BLASTX | hypothetical protein F6E13.3 - | PIR: T00670 | 2e−26 |
| | | orf | Arabidopsis thaliana | | |
| 116 | km3f_t23-c03r | BLASTX | gene: “Nt-SubE80”; Nicotiana tabacum | TREMBL: AB041515_1 | 2e−05 |
| | | orf | Nt-SubE80 mRNA, complete cds. | | |
| 118 | km3f_t23-c05r | BLASTX | product: “mitochondrial protein-like | TREMBLNEW: AB047269_1 | 1e−05 |
| | | orf | protein”; Cucumis sativus mRNA for | | |
| | | | mitochondrial protein-like protein, partial | | |
| | | | cds. | | |
| 119 | km3f_t23-c06r | BLASTX | hypothetical protein T26I12.30 - | PIR: T47654 | 2e−10 |
| | | orf | Arabidopsis thaliana | | |
| 120 | km3f_t23-c07r | BLASTX | ABC-type transport protein homolog | PIR: T02644 | 8e−06 |
| | | orf | F12C20.5 - Arabidopsis thaliana | | |
| 124 | km3f_t23-c11r | BLASTX | Arabidopsis thaliana genomic DNA, | TREMBL: AB025628_12 | 8e−18 |
| | | orf | chromosome 5, P1 clone: MNJ7. | | |
| 125 | km3f_t23-c12r | BLASTX | gene: “F22M8.4”; Sequence of BAC | TREMBL: AC020622_4 | 1e−07 |
| | | orf | F22M8 from Arabidopsis thaliana | | |
| | | | chromosome 1, complete sequence. | | |
| 126 | km3f_t23-d01r | BLASTX | callose synthase catalytic subunit-like | PIR: T49914 | 4e−11 |
| | | orf | protein - Arabidopsis thaliana | | |
| 127 | km3f_t23-d02r | BLASTX | photosystem II protein D1 precursor - | PIR: S33912 | 2e−12 |
| | | orf | southern Asian dodder chloroplast | | |
| 129 | km3f_t23-d04r | BLASTX | gene: “CYP81B11”; product: | TREMBL: HTCYP81L_1 | 2e−31 |
| | | orf | “cytochrome P450”; Helianthus | | |
| | | | tuberosus mRNA for cytochrome P450, | | |
| | | | CYP81B11 | | |
| 134 | km3f_t23-d09r | BLASTX | receptor-protein kinase-like protein - | PIR: T45786 | 4e−25 |
| | | orf | Arabidopsis thaliana | | |
| 135 | km3f_t23-d10r | BLASTX | gene: “Td”; product: “threonine | TREMBL: LEILV1A_1 | 3e−51 |
| | | orf | deaminase”; L. esculentum threonine | | |
| | | | deaminase (Td) mRNA, 3′ end. | | |
| 137 | km3f_t23-d12r | BLASTX | hypothetical protein T8P19.200 - | PIR: T46213 | 5e−05 |
| | | orf | Arabidopsis thaliana | | |
| 138 | km3f_t23-e01r | BLASTX | cysteine-rich extensin-like protein 2 | PIR: B48232 | 2e−19 |
| | | orf | precursor - common tobacco | | |
| 140 | km3f_t23-e03r | BLASTX | gene: “F11F12.8”; product: “Putative | TREMBL: AC012561_8 | 6e−20 |
| | | orf | transcription factor”Arabidopsis | | |
| | | | thaliana chromosome I BAC F11F12 | | |
| | | | genomic sequence, complete sequence. | | |
| 142 | km3f_t23-e05r | BLASTX | gene: “atpB”; product: “ATP synthase | TREMBLNEW: FUT400885_1 | 9e−05 |
| | | orf | subunit B”; Fendlerella utahensis partial | | |
| | | | atpB gene for ATP synthase subunit B | | |
| 143 | km3f_t23-e06r | BLASTX | hypothetical protein T26I12.30 - | PIR: T47654 | 4e−09 |
| | | orf | Arabidopsis thaliana | | |
| 144 | km3f_t23-e07r | BLASTX | cytochrome P450 71A1 - avocado | PIR: A35867 | 4e−14 |
| | | orf | | | |
| 147 | km3f_t23-e10r | BLASTX | hypothetical protein T5N23.70 - | PIR: T47630 | 4e−10 |
| | | orf | Arabidopsis thaliana | | |
| 153 | km3f_t23-f04r | BLASTX | product: “PDR5-like ABC transporter”; | TREMBL: SPPDR5ABC_1 | 2e−40 |
| | | orf | S. polyrrhiza mRNA for PDR5-like ABC | | |
| | | | transporter | | |
| 154 | km3f_t23-f05r | BLASTX | GLUTAMATE DECARBOXYLASE 2 | SWISSPROT: DCE2_ARATH | 2e−34 |
| | | orf | (EC 4.1.1.15) (GAD 2). | | |
| 156 | km3f_t23-f07r | BLASTX | hypothetical protein F20D10.300 - | PIR: T05645 | 3e−08 |
| | | orf | Arabidopsis thaliana | | |
| 157 | km3f_t23-f08r | BLASTX | product: “RNA-binding protein-like”; | TREMBL: AB026647_9 | 3e−17 |
| | | orf | Arabidopsis thaliana genomic DNA, | | |
| | | | chromosome 3, P1 clone: MJL12. | | |
| 158 | km3f_t23-f09r | BLASTX | proteinase inhibitor I precursor - tomato | PIR: A24048 | 6e−22 |
| | | orf | | | |
| 160 | km3f_t23-f11r | Longest orf | gene: “F17O14.25”; product: | TREMBL: AC012562_25 | 0.21 |
| | | | “hypothetical protein; 78375-76401”; | | |
| | | | Arabidopsis thaliana chromosome 3 | | |
| | | | BAC F17O14 genomic sequence, | | |
| | | | complete sequence. | | |
| 161 | km3f_t23-f12r | BLASTX | product: “polymerase”; Rice ragged stunt | TREMBL: AF015682_1 | 5e−05 |
| | | orf | virus polymerase mRNA, complete cds. | | |
| 165 | km3f_t23-g04r | BLASTX | STH 2 protein - potato | PIR: S35161 | 1e−33 |
| | | orf | | | |
| 166 | km3f_t23-g05r | BLASTX | product: “hygromycin | TREMBL: AF234292_2 | 3e−38 |
| | | orf | phosphotransferase”; Binary vector | | |
| | | | pCAMBIA-1200, complete sequence. | | |
| 168 | km3f_t23-g07r | BLASTX | gene: “At2g15780”; Arabidopsis thaliana | TREMBL: AC006438_3 | 7e−10 |
| | | orf | chromosome II section 92 of 255 of the | | |
| | | | complete sequence. Sequence from | | |
| | | | clones F19G14, F7H1. | | |
| 169 | km3f_t23-g08r | BLASTX | hypothetical protein F25G13.100 - | PIR: T10203 | 7e−21 |
| | | orf | Arabidopsis thaliana | | |
| 170 | km3f_t23-g09r | BLASTX | callose synthase catalytic subunit-like | PIR: T49914 | 4e−11 |
| | | orf | protein - Arabidopsis thaliana | | |
| 172 | km3f_t23-g11r | BLASTX | product: “T7N9.26”; Genomic sequence | TREMBL: ATAC348_26 | 3e−15 |
| | | orf | for Arabidopsis thaliana BAC T7N9 | | |
| | | | from chromosome I, complete sequence. | | |
| 173 | km3f_t23-g12r | BLASTX | gene: “T11I18.5”; product: “putative | TREMBL: AC011698_5 | 2e−10 |
| | | orf | casein kinase”; Arabidopsis thaliana | | |
| | | | chromosome III BAC T11I18 genomic | | |
| | | | sequence, complete sequence. | | |
| 175 | km3f_t23-h02r | BLASTX | Arabidopsis thaliana genomic DNA, | TREMBL: AB025628_12 | 3e−19 |
| | | orf | chromosome 5, P1 clone: MNJ7. | | |
| 178 | km3f_t23-h05r | BLASTX | hypothetical protein F18B3.220 - | PIR: T08415 | 5e−11 |
| | | orf | Arabidopsis thaliana | | |
| 180 | km3f_t23-h07r | BLASTX | receptor-protein kinase-like protein - | PIR: T45786 | 5e−24 |
| | | orf | Arabidopsis thaliana | | |
| 183 | km3f_t23-h11r | BLASTX | gene: “Lhcb1*7”; product: “light | TREMBL: AB012639_1 | 5e−09 |
| | | orf | harvesting chlorophyll a/b-binding | | |
| | | | protein”Nicotiana sylvestris Lhcb1*7 | | |
| | | | gene for light harvesting chlorophyll a/b- | | |
| | | | binding protein, complete cds. | | |
| 40 | km3f_tr11-11r | BLASTX | cathepsin D inhibitor precursor - potato | PIR: S52656 | 5e−21 |
| | | orf | | | |
| 42 | km3f_tr11-15r | BLASTX | gene: “CBP4”; product: “cyclic | TREMBL: AF079872_1 | 5e−09 |
| | | orf | nucleotide-gated calmodulin-binding ion | | |
| | | | channel”; Nicotiana tabacum cyclic | | |
| | | | nucleotide-gated calmodulin-binding ion | | |
| | | | channel (CBP4) mRNA, complete cds. | | |
| 43 | km3f_tr11-18r | BLASTX | receptor protein kinase-like protein - | PIR: T45899 | 1e−08 |
| | | orf | Arabidopsis thaliana | | |
| 46 | km3f_tr11-22r | BLASTX | gene: “RKL1”; product: “receptor-like | TREMBL: AF084034_1 | 7e−06 |
| | | orf | protein kinase”; Arabidopsis thaliana | | |
| | | | receptor-like protein kinase (RKL1) | | |
| | | | gene, complete cds. | | |
| 48 | km3f_tr11-26r | BLASTX | mRNA-binding protein precursor | PIRNEW: T52071 | 5e−74 |
| | | orf | [imported] - tomato (fragment) | | |
| 49 | km3f_tr11-29r | BLASTX | hypothetical protein F14P22.120 - | PIR: T45673 | 0.006 |
| | | orf | Arabidopsis thaliana | | |
| 50 | km3f_tr11-2r | BLASTX | ribosomal protein L4, chloroplast - | PIR: T01739 | 5e−17 |
| | | orf | common tobacco | | |
| 52 | km3f_tr11-33r | BLASTX | gene: “P0665D10.14”; Oryza sativa | TREMBL: AP002861_14 | 2e−07 |
| | | orf | genomic DNA, chromosome 1, PAC | | |
| | | | clone: P0665D10. | | |
| 56 | km3f_tr11-44r | Longest orf | SLP1 protein homolog - Caenorhabditis | PIR: S27790 | 0.21 |
| | | | elegans | | |
| 57 | km3f_tr11-49r | BLASTX | Arabidopsis thaliana genomic DNA, | TREMBL: AB025628_12 | 8e−27 |
| | | orf | chromosome 5, P1 clone: MNJ7. | | |
| 58 | km3f_tr11-4r | BLASTX | probable cytochrome P450, | PIR: T03275 | 4e−11 |
| | | orf | hypersensitivity-related - common | | |
| | | | tobacco | | |
| 60 | km3f_tr11-53r | BLASTX | Cf-4A protein - tomato | PIR: T07015 | 1e−52 |
| | | orf | | | |
| 61 | km3f_tr11-54r | BLASTX | product: “ubiquitin carboxyl-terminal | TREMBL: AP002543_13 | 4e−04 |
| | | orf | hydrolase”; Arabidopsis thaliana | | |
| | | | genomic DNA, chromosome 5, BAC | | |
| | | | clone: F15M7. | | |
| 64 | km3f_tr11-58r | BLASTX | Oryza sativa genomic DNA, | TREMBL: AP001081_21 | 3e−07 |
| | | orf | chromosome 1, clone: P0693B08. | | |
| 65 | km3f_tr11-61r | BLASTX | gene: “At2g26190”; Arabidopsis thaliana | TREMBL: AC004484_4 | 1e−50 |
| | | orf | chromosome II section 147 of 255 of the | | |
| | | | complete sequence. Sequence from | | |
| | | | clones T19L18, T1D16, T9J22. | | |
| 66 | km3f_tr11-68r | BLASTX | product: “receptor-like protein kinase”; | TREMBL: AB019228_17 | 4e−28 |
| | | orf | Arabidopsis thaliana genomic DNA, | | |
| | | | chromosome 5, P1 clone: MCK7. | | |
| 70 | km3f_tr11-74r | BLASTX | gene: “aak1”; product: “putative serine | TREMBL: ATH242671_1 | 2e−26 |
| | | orf | threonine kinase”; Arabidopsis thaliana | | |
| | | | mRNA for putative serine threonine | | |
| | | | kinase (aak1 gene) | | |
| 71 | km3f_tr11-77r | BLASTX | histone H4 - tomato | PIR: S32769 | 1e−18 |
| | | orf | | | |
| 73 | km3f_tr11-7r | BLASTX | product: “protein disulphide isomerase- | TREMBL: ATAB5246_6 | 2e−14 |
| | | orf | like protein”; Arabidopsis thaliana | | |
| | | | genomic DNA, chromosome 5, P1 | | |
| | | | clone: MUP24. | | |
| 80 | km3f_tr11-91r | BLASTX | gene: “PAT1”; product: “patatin-like | TREMBL: AF158027_1 | 6e−08 |
| | | orf | protein 1”; Nicotiana tabacum patatin- | | |
| | | | like protein 1 (PAT1) mRNA, complete | | |
| | | | cds. | | |
| 88 | km3f_tr1114-90r | BLASTX | gene: “At2g26190”; Arabidopsis thaliana | TREMBL: AC004484_4 | 1e−46 |
| | | orf | chromosome II section 147 of 255 of the | | |
| | | | complete sequence. Sequence from | | |
| | | | clones T19L18, T1D16, T9J22. | | |
| 89 | km3f_tr1114-92r | BLASTX | Arabidopsis thaliana genomic DNA, | TREMBL: AB025628_12 | 8e−31 |
| | | orf | chromosome 5, P1 clone: MNJ7. | | |
| 90 | km3f_tr1114-94r | BLASTX | MAP kinase [imported] - Arabidopsis | PIRNEW: T51099 | 7e−06 |
| | | orf | thaliana | | |
| 345 | km3f_tr15-14r | BLASTX | nitrate reductase (NADH) (EC 1.6.6.1) - | PIR: RDTONH | 2e−06 |
| | | orf | tomato | | |
| 346 | km3f_tr15-15r | BLASTX | gene: “GUS/NPT-II fusion”; product: | TREMBL: AGGUNPIIF_1 | 9e−15 |
| | | orf | “beta-glucuronidase: neomycin | | |
| | | | phosphotransferase II fusion protein”; | | |
| | | | Synthetic E. coli beta- | | |
| | | | glucuronidase/neomycin | | |
| | | | phosphotransferase II fusion protein | | |
| | | | (GUS/NPT-II) gene, 5′ end. | | |
| 347 | km3f_tr15-16r | BLASTX | product: “GUSA (N358Q); hexaHis | TREMBL: AF234302_1 | 8e−16 |
| | | orf | tagged”; Binary vector pCAMBIA-1381, | | |
| | | | complete sequence. | | |
| 348 | km3f_tr15-18r | BLASTX | hypothetical protein TC0130 [imported] - | PIR: G81737 | 5e−11 |
| | | orf | Chlamydia muridarum (strain Nigg) | | |
| 349 | km3f_tr15-19r | BLASTX | leucyl aminopeptidase (EC 3.4.11.1) | PIR: T07850 | 3e−10 |
| | | orf | (clone pBlap2) precursor, wound- | | |
| | | | induced - tomato (fragment) | | |
| 355 | km3f_tr15-25r | BLASTX | gene: “petB”; product: “cytochrome b6”; | TREMBL: OEL271079_67 | 2e−17 |
| | | orf | Oenothera elata subsp. hookeri | | |
| | | | chloroplast plastome I, complete | | |
| | | | sequence | | |
| 356 | km3f_tr15-26r | Longest orf | hypothetical protein PH0220 - | PIR: B71245 | 0.46 |
| | | | Pyrococcus horikoshii | | |
| 357 | km3f_tr15-27r | BLASTX | hypothetical protein F25G13.100 - | PIR: T10203 | 5e−16 |
| | | orf | Arabidopsis thaliana | | |
| 358 | km3f_tr15-29r | BLASTX | mRNA-binding protein precursor | PIRNEW: T52071 | 5e−41 |
| | | orf | [imported] - tomato (fragment) | | |
| 359 | km3f_tr15-2r | BLASTX | phosphoinositide-specific phospholipase | PIR: T07424 | 5e−11 |
| | | orf | C (EC 3.1.4.—) plc2 - potato | | |
| 362 | km3f_tr15-32r | BLASTX | AVR4 protein - fungus (Cladosporium | PIR: S41047 | 1e−11 |
| | | orf | fulvum) | | |
| 363 | km3f_tr15-33r | BLASTX | product: “F3M18.17”; Genomic | TREMBL: AC010155_17 | 6e−05 |
| | | orf | sequence for Arabidopsis thaliana BAC | | |
| | | | F3M18 from chromosome I, complete | | |
| | | | sequence. | | |
| 364 | km3f_tr15-34r | BLASTX | hypothetical protein F9G14.160 - | PIR: T48306 | 3e−16 |
| | | orf | Arabidopsis thaliana | | |
| 365 | km3f_tr15-35r | BLASTX | hypothetical protein F9G14.160 - | PIR: T48306 | 3e−16 |
| | | orf | Arabidopsis thaliana | | |
| 366 | km3f_tr15-36r | BLASTX | Arabidopsis thaliana genomic DNA, | TREMBL: AB020747_4 | 2e−09 |
| | | orf | chromosome 5, P1 clone: MPI10. | | |
| 367 | km3f_tr15-37r | BLASTX | gene: “At2g06990”; Arabidopsis thaliana | TREMBL: AC005171_6 | 3e−17 |
| | | orf | chromosome II section 39 of 255 of the | | |
| | | | complete sequence. Sequence from | | |
| | | | clones T9F8, T4E14. | | |
| 369 | km3f_tr15-39r | BLASTX | gene: “At2g18150”; product: “putative | TREMBL: AC007212_5 | 4e−09 |
| | | orf | peroxidase”; Arabidopsis thaliana | | |
| | | | chromosome II section 106 of 255 of the | | |
| | | | complete sequence. Sequence from | | |
| | | | clones F8D23, T30D6. | | |
| 370 | km3f_tr15-3r | BLASTX | Oryza sativa genomic DNA, | TREMBL: AP002481_22 | 2e−30 |
| | | orf | chromosome 1, clone: P0702F03. | | |
| 371 | km3f_tr15-40r | BLASTX | product: “glutamine synthetase”; | TREMBL: LE14754_1 | 3e−14 |
| | | orf | Lycopersicon esculentum glutamine | | |
| | | | synthetase mRNA, partial cds. | | |
| 373 | km3f_tr15-42r | BLASTX | gene: “F13F21.5”; Arabidopsis thaliana | TREMBL: AC007504_5 | 8e−05 |
| | | orf | chromosome I BAC F13F21 genomic | | |
| | | | sequence, complete sequence. | | |
| 374 | km3f_tr15-43r | BLASTX | gene: “F16P17.17”; Sequence of BAC | TREMBL: AC011000_17 | 2e−07 |
| | | orf | F16P17 from Arabidopsis thaliana | | |
| | | | chromosome 1, complete sequence. | | |
| 375 | km3f_tr15-44r | BLASTX | hypothetical protein T22P22.140 - | PIR: T48534 | 2e−18 |
| | | orf | Arabidopsis thaliana | | |
| 377 | km3f_tr15-46r | BLASTX | leucyl aminopeptidase (EC 3.4.11.1) | PIR: T07850 | 7e−11 |
| | | orf | (clone pBlap2) precursor, wound- | | |
| | | | induced - tomato (fragment) | | |
| 378 | km3f_tr15-47r | BLASTX | gene: “F21M12.36”; Sequence of BAC | TREMBL: ATAC132_35 | 4e−06 |
| | | orf | F21M12 from Arabidopsis thaliana | | |
| | | | chromosome 1, complete sequence. | | |
| 380 | km3f_tr15-4r | BLASTX | hypothetical protein F19F18.160 - | PIR: T04724 | 3e−08 |
| | | orf | Arabidopsis thaliana | | |
| 381 | km3f_tr15-50r | BLASTX | hypothetical protein F25G13.100 - | PIR: T10203 | 4e−09 |
| | | orf | Arabidopsis thaliana | | |
| 382 | km3f_tr15-51r | BLASTX | hypothetical protein F25G13.100 - | PIR: T10203 | 1e−08 |
| | | orf | Arabidopsis thaliana | | |
| 383 | km3f_tr15-52r | BLASTX | gene: “At2g38610”; product: “putative | TREMBL: AC00 [symbol] | [symbol] |
| | | orf | RNA-binding protein”Arabidopsis | | |
| | | | thaliana chromosome II section 208 of | | |
| | | | 255 of the complete sequence. Sequence | | |
| | | | from clones T19C21, T6A23. | | |
| 391 | km3f_tr15-61r | BLASTX | S-receptor kinase (EC 2.7.1.—) | PIR: T05179 | 1e−05 |
| | | orf | T6K22.100 precursor - Arabidopsis | | |
| | | | thaliana | | |
| 392 | km3f_tr15-62r | BLASTX | gene: “F18B13.10”; Arabidopsis thaliana | TREMBL: AC009322_9 | 3e−04 |
| | | orf | chromosome I BAC F18B13 genomic | | |
| | | | sequence, complete sequence. | | |
| 393 | km3f_tr15-63r | BLASTX | gene: “MS”; product: “methionine | TREMBL: AF082893_1 | 1e−31 |
| | | orf | synthase”; Solanum tuberosum | | |
| | | | methionine synthase (MS) mRNA, | | |
| | | | complete cds. | | |
| 398 | km3f_tr15-68r | BLASTX | gene: “F12F1.28”; Arabidopsis thaliana | TREMBL: ATAC2131_28 | 1e−12 |
| | | orf | chromosome 1 BAC F12F1 sequence, | | |
| | | | complete sequence. | | |
| 400 | km3f_tr15-6r | BLASTX | gene: “At2g07680”; product: “putative | TREMBL: AC006225_1 | 2e−12 |
| | | orf | ABC transporter”; Arabidopsis thaliana | | |
| | | | chromosome II section 47 of 255 of the | | |
| | | | complete sequence. Sequence from | | |
| | | | clones T5E7, T12J2. | | |
| 401 | km3f_tr15-70r | BLASTX | gene: “psbC”; product: “photosystem II | TREMBL: AF188854_2 | 1e−05 |
| | | orf | CP43 protein”; Nymphaea odorata | | |
| | | | photosystem II D2 protein (psbD) and | | |
| | | | photosystem II CP43 protein (psbC) | | |
| | | | genes, partial cds; chloroplast genes for | | |
| | | | chloroplast products. | | |
| 403 | km3f_tr15-72r | BLASTX | gene: “Glu-R1”; product: “glutenin, high | TREMBL: AF216868_1 | 1e−04 |
| | | orf | molecular weight subunit type x | | |
| | | | precursor”; Triticum aestivum cultivar | | |
| | | | 7841 glutenin, high molecular weight | | |
| | | | subunit type x precursor (Glu-R1) gene, | | |
| | | | Glu-R1x allele, complete cds. | | |
| 407 | km3f_tr15-76r | BLASTX | catechol oxidase (EC 1.10.3.1) precursor | PIR: S33544 | 7e−30 |
| | | orf | [similarity] - tomato | | |
| 411 | km3f_tr15-7r | BLASTX | shaggy protein kinase 4 (EC 2.7.1.—) - | PIR: S51105 | 3e−06 |
| | | orf | garden petunia | | |
| 415 | km3f_tr15-83r | BLASTX | hypothetical protein F25G13.100 - | PIR: T10203 | 4e−09 |
| | | orf | Arabidopsis thaliana | | |
| 417 | km3f_tr15-85r | BLASTX | hypothetical protein F25G13.100 - | PIR: T10203 | 4e−09 |
| | | orf | Arabidopsis thaliana | | |
| 418 | km3f_tr15-88r | BLASTX | hypothetical protein F6E13.8 - | PIR: T00675 | 5e−04 |
| | | orf | Arabidopsis thaliana | | |
| 419 | km3f_tr15-8r | BLASTX | hypothetical protein T22P22.140 - | PIR: T48534 | 5e−20 |
| | | orf | Arabidopsis thaliana | | |
| 420 | km3f_tr15-9r | BLASTX | product: “putative ammonium transporter | TREMBL: AF182188_1 | 3e−17 |
| | | orf | AMT1; 1”; Lotus japonicus putative | | |
| | | | ammonium transporter AMT1; 1 mRNA, | | |
| | | | complete cds. | | |
| 277 | km3f_trest-a03r | BLASTX | product: “peptidylprolyl isomerase; | TREMBL: AB026647_19 | 1e−04 |
| | | orf | FK506-binding protein”; Arabidopsis | | |
| | | | thaliana genomic DNA, chromosome 3, | | |
| | | | P1 clone: MJL12. | | |
| 278 | km3f_trest-a04r | BLASTX | product: “F12K21.25”; Genomic | TREMBL: AC023279_25 | 0.002 |
| | | orf | sequence for Arabidopsis thaliana BAC | | |
| | | | F12K21 from chromosome I, complete | | |
| | | | sequence. | | |
| 281 | km3f_trest-a07r | BLASTX | nuclear receptor binding factor-like | PIR: T47517 | 3e−05 |
| | | orf | protein - Arabidopsis thaliana | | |
| 282 | km3f_trest-a08r | BLASTX | omega-6 fatty acid desaturase (EC | PIR: T07009 | 3e−41 |
| | | orf | 1.14.99.—) defense-related - tomato | | |
| 283 | km3f_trest-a09r | BLASTX | nuclear receptor binding factor-like | PIR: T47517 | 2e−11 |
| | | orf | protein - Arabidopsis thaliana | | |
| 284 | km3f_trest-a10r | BLASTX | nuclear receptor binding factor-like | PIR: T47517 | 5e−05 |
| | | orf | protein - Arabidopsis thaliana | | |
| 285 | km3f_trest-a11r | BLASTX | product: “nucleotide sugar epimerase- | TREMBL: AP001297_1 | 0.005 |
| | | orf | like protein”; Arabidopsis thaliana | | |
| | | | genomic DNA, chromosome 3, BAC | | |
| | | | clone: F14O13. | | |
| 286 | km3f_trest-a12r | BLASTX | cathepsin D inhibitor pdi - potato | PIR: XKPODC | 1e−13 |
| | | orf | | | |
| 288 | km3f_trest-b02r | BLASTX | gene: “F9E11.4”; product: “disease | TREMBL: AC079678_1 | 5e−11 |
| | | orf | resistance protein, putative; 1096-4664”; | | |
| | | | Arabidopsis thaliana chromosome 1 | | |
| | | | BAC F9E11 genomic sequence, | | |
| | | | complete sequence. | | |
| 290 | km3f_trest-b04r | BLASTX | hypothetical protein T20K18.180 - | PIR: T06641 | 1e−21 |
| | | orf | Arabidopsis thaliana | | |
| 291 | km3f_trest-b05r | BLASTX | photosystem II protein W homolog | PIR: T10660 | 3e−29 |
| | | orf | T5F17.110 - Arabidopsis thaliana | | |
| 295 | km3f_trest-b10r | BLASTX | probable cinnamyl-alcohol | PIR: T16995 | 7e−25 |
| | | orf | dehydrogenase (EC 1.1.1.195) - apple | | |
| | | | tree | | |
| 301 | km3f_trest-c04r | BLASTX | gene: “T16N11.3”; product: “Putative | TREMBL: AC013453_3 | 5e−18 |
| | | orf | ABC transporter”; Arabidopsis thaliana | | |
| | | | chromosome I BAC T16N11 genomic | | |
| | | | sequence, complete sequence. | | |
| 302 | km3f_trest-c05r | BLASTX | Arabidopsis thaliana genomic DNA, | TREMBL: AP001307_10 | 2e−07 |
| | | orf | chromosome 3, P1 clone: MMM17. | | |
| 304 | km3f_trest-c07r | Longest orf | nucleoid DNA-binding protein cnd41- | PIR: T50786 | 0.017 |
| | | | like protein - Arabidopsis thaliana | | |
| 305 | km3f_trest-c08r | BLASTX | gene: “P69E”; product: “subtilisin-like | TREMBL: LES18931_1 | 7e−09 |
| | | orf | protease”; Lycopersicon esculentum | | |
| | | | p69E gene | | |
| 306 | km3f_trest-c09r | BLASTX | gene: “PH1”; product: “anthocyanin 5-O- | TREMBL: AB027455_1 | 5e−20 |
| | | orf | glucosyltransferase”; Petunia x hybrida | | |
| | | | PH1 mRNA for anthocyanin 5-O- | | |
| | | | glucosyltransferase, complete cds. | | |
| 310 | km3f_trest-d02r | BLASTX | gene: “At2g02090”; product: | TREMBL: AC005936_3 | 4e−20 |
| | | orf | “SNF2/RAD54 family (ETL1 subfamily) | | |
| | | | protein”; Arabidopsis thaliana | | |
| | | | chromosome II section 8 of 255 of the | | |
| | | | complete sequence. Sequence from | | |
| | | | clones F14H20, F5O4. | | |
| 312 | km3f_trest-d04r | BLASTX | gene: “gln”; product: “glutamine | TREMBLNEW: AF302113_1 | 5e−06 |
| | | orf | synthetase GS2”; Solanum tuberosum | | |
| | | | glutamine synthetase GS2 (gln) mRNA, | | |
| | | | partial cds; nuclear gene for plastid | | |
| | | | product. | | |
| 316 | km3f_trest-d09r | BLASTX | gene: “F5E6.5”; product: “protein kinase, | TREMBL: AC020580_5 | 3e−08 |
| | | orf | putative; 19229-23534”Arabidopsis | | |
| | | | thaliana chromosome 3 BAC F5E6 | | |
| | | | genomic sequence, complete sequence. | | |
| 317 | km3f_trest-d10r | BLASTX | subtilisin-like proteinase (EC 3.4.21.—) | PIR: T06579 | 2e−18 |
| | | orf | p69e - tomato | | |
| 318 | km3f_trest-d12r | BLASTX | gene: “p69d”; product: “serine protease”; | TREMBL: LES6786_1 | 5e−12 |
| | | orf | Lycopersicon esculentum p69d gene | | |
| 322 | km3f_trest-e04r | BLASTX | hypothetical protein F21B7.8 - | PIR: T00894 | 2e−06 |
| | | orf | Arabidopsis thaliana | | |
| 323 | km3f_trest-e05r | BLASTX | probable glucose-6- | PIR: T06997 | 7e−23 |
| | | orf | phosphate/phosphate-translocator | | |
| | | | precursor - potato (fragment) | | |
| 324 | km3f_trest-e06r | BLASTX | product: “cytochrome P450”; Pisum | TREMBLNEW: AF218296_1 | 6e−16 |
| | | orf | sativum cytochrome P450 mRNA, | | |
| | | | complete cds. | | |
| 325 | km3f_trest-e07r | BLASTX | heat shock protein 80 - tomato | PIR: T07037 | 2e−36 |
| | | orf | | | |
| 326 | km3f_trest-e08r | BLASTX | leucyl aminopeptidase (EC 3.4.11.1) | PIR: T07850 | 7e−11 |
| | | orf | (clone pBlap2) precursor, wound- | | |
| | | | induced - tomato (fragment) | | |
| 327 | km3f_trest-e09r | Longest orf | acetyltransferase, putative VCA0470 | PIRNEW: F82455 | 0.039 |
| | | | [imported] - Vibrio cholerae (group O1 | | |
| | | | strain N16961) | | |
| 329 | km3f_trest-e11r | BLASTX | gene: “AT4g08850”; product: “receptor | TREMBL: ATCHRIV25_4 | 3e−04 |
| | | orf | protein kinase-like protein”Arabidopsis | | |
| | | | thaliana DNA chromosome 4, contig | | |
| | | | fragment No. 25 | | |
| 330 | km3f_trest-f01r | BLASTX | hypothetical protein T8P19.200 - | PIR: T46213 | 5e−05 |
| | | orf | Arabidopsis thaliana | | |
| 336 | km3f_trest-f07r | BLASTX | product: “F15H18.11”; Genomic | TREMBL: AC013354_11 | 2e−07 |
| | | orf | sequence for Arabidopsis thaliana BAC | | |
| | | | F15H18 from chromosome I, complete | | |
| | | | sequence. | | |
| 337 | km3f_trest-f08r | BLASTX | CATALASE ISOZYME 1 (EC 1.11.1.6). | SWISSPROT: CAT1_LYCES | 3e−14 |
| | | orf | | | |
| 338 | km3f_trest-f09r | BLASTX | leucyl aminopeptidase (EC 3.4.11.1) | PIR: T07850 | 5e−16 |
| | | orf | (clone pBlap2) precursor, wound- | | |
| | | | induced - tomato (fragment) | | |
| 339 | km3f_trest-f11r | BLASTX | subtilisin-like proteinase (EC 3.4.21.—) | PIR: T07184 | 1e−25 |
| | | orf | precursor P69B, pathogenesis-related - | | |
| | | | tomato | | |

TABLE 7
Differentials annotated to the various superfamilies

| Superfamily | Reference |
|---|---|
| 1-phosphatidyl- | Kopka, J.; Pical, C.; Gray, J. E.; Mueller-Roeber, B. Plant Physiol. 116, |
| inositol-4,5- | 239-250 (1998) Molecular and enzymatic characterization of three |
| bisphosphate | phosphoinositide-specific phospholipase C isoforms from potato. |
| phosphodiesterase | |
| Alcohol | Bevan, M.; Pohl, T.; Weizenegger, T.; Bancroft, I.; Mewes, H. W.; |
| dehydrogenase; | Mayer, K. F. X.; Lemcke, K.; Schueller, C. submitted to the Protein |
| | Sequence Database, June 1999 |
| Branched-chain | Popov, K. M.; Zhao, Y.; Shimomura, Y.; Kuntz, M. J.; Harris, R. A. J. |
| alpha-keto acid | Biol. Chem. 267, 13127-13130 (1992) Branched-chain alpha-ketoacid |
| dehydrogenase | dehydrogenase kinase. Molecular cloning, expression, and sequence |
| kinase | similarity with histidine protein kinases. |
| Catechol oxidase | Newman, S. M.; Eannetta, N. T.; Yu, H.; Prince, J. P.; de Vicente, C.; |
| | Tanksley, S. D.; Steffens, J. C. Plant Mol. Biol. 21, 1035-1051 (1993) |
| | Organisation of the tomato polyphenol oxidase gene family. |
| Cathepsin d | Herbers, K.; Prat, S.; Willmitzer, L. Plant Mol. Biol. 26, 73-83, (1994): |
| inhibitor: | Cloning and characterization of a cathepsin D inhibitor gene from |
| | Solanum tuberosum L. |
| Cytochrome b6 | Sato S., Nakamura Y., Kaneko T., Asamizu E., Tabata S.; DNA Res. |
| | 6: 283-290(1999). Complete structure of the chloroplast genome of |
| | Arabidopsis thaliana |
| Cytochrome p450 | Czernic, P.; Huang, H. C.; Marco, Y. Plant Mol. Biol. 31, 255-265, |
| homology | (1996) Characterization of hsr201 and hsr515, two tobacco genes |
| | preferentially expressed during the HR provoked by phytopathogenic |
| | bacteria. |
| Cytosol | Gu, Y.; Chao, W. S.; Walling, L. L. J. Biol. Chem. 271, 25880-25887, |
| aminopeptidase | (1996) Localization and post-translational processing of the wound- |
| | induced leucine aminopeptidase of tomato. |
| Dna binding | Vian A., Henry-Vian C., Davies E.; Plant Physiol. 121(2): 517-524 |
| | (1999). Rapid and systemic accumulation of chloroplast mRNA- |
| | binding protein transcripts after flame stimulus in tomato |
| Dnaj amino-terminal | Benes, V.; Rechmann, S.; Borkova, D.; Ansorge, W.; Mewes, H. W.; |
| homology | Lemcke, K.; Mayer, K. F. X.; Quetier, F.; Salanoubat, M. submitted to the |
| | Protein Sequence Database, January 2000 |
| Carbonate | Yamada S., Komori T., Kubo T., Imasaki H.; Plant Physiol. 117: 1126-1126 |
| dehydratase | (1998). A cDNA clone for salt-stress-induced carbonic anhydrase |
| | from Nicotiana paniculata (Accession No. AB012863) (PGR98-124)” |
| Ribosomal protein 14 | Yokoi, F.; Ohta, M.; Sugiura, M. submitted to the EMBL Data Library, |
| | January 1998A; Description: Tobacco chloroplast ribosomal protein L4. |
| Ferredoxin 2[4fe-4s] | Lin, C. H.; Wu, M. lant Mol. Biol. 15, 449-455 (990) A ferredoxin-type |
| | iron-sulfur protein gene, frx B, is expressed in the chloroplasts of |
| | tobacco and spinach. |
| Glutamate- | Perez-Rodriguez J., Valpuesta V.; Expression of glutamine synthetase- |
| ammonia ligase | genes during expansion and senescence of tomato leaves; Unpublished. |
| Histone h4 | Brandstaedter J. U., Rossbach C., Thers K.; Planta 192: 69-74 (1994). The |
| | pattern of histone H4 expression in the tomato shoot apex changes uring |
| | development |
| Kinase-related | Ito, Y.; Banno, H.; Moribe, T.; Hinata, K.; Machida, K. H. Y. Mol. Gen. |
| transforming protein | Genet. 245, 1-10 (1994): NPK15, a tobacco protein-serine/threonine |
| | kinase with a single hydrophobic region near the amino-terminus. |
| Leucine-rich alpha- | Takken, F. L.; Schipper, D.; Nijkamp, H. J.; Hille, J. Plant J. 14, 401-411, |
| 2-glycoprotein | (1998) Identification and Ds-tagged isolation of a new gene at the Cf-4 |
| repeat homology | locus of tomato involved in disease resistance to Cladosporium fulvum |
| | race 5. |
| Peroxidase | Osakabe, K.; Kawai, S.; Katayama, Y.; Morohoshi, N. submitted to the |
| | EMBL Data Library, June 1992 Description: Nucleotide sequence for |
| | the genomic DNA encoding the anionic peroxidase gene from Nicotiana |
| | tabacum. |
| | Roberts, E.; Kutchan, T.; Kolattukudy, P. E. Plant Mol. Biol. 11, 15-26 |
| | (1988) Cloning and sequencing of cDNA for a highly anionic |
| | peroxidase from potato and the induction of its mRNA in suberizing |
| | potato tubers and tomato fruits. |
| Protein disulfide- | Quetier, F.; Choisne, N.; Robert, C.; Brottier, P.; Wincker, P.; Cattolico, L.; |
| isomerase | Artiguenave, F.; Saurin, W.; Weissenbach, J.; Salanoubat, M.; |
| | Mewes, H. W.; Mayer, K. F. X.; Schueller, C. Submitted to the Protein |
| | Sequence Database, April 1999 |
| Protein kinase | Bevan, M.; Peters, S. A.; van Staveren, M.; Dirkse, W.; Stiekema, W.; |
| homology | Bancroft, I.; Mewes, H. W.; Mayer, K. F. X.; Schueller, C. Submitted to |
| | the Protein Sequence Database, August 1998 |
| Protein kinase xa21 | Choisne, N.; Robert, C.; Brottier, P.; Wincker, P.; Cattolico, L.; |
| | Artiguenave, F.; Saurin, W.; Weissenbach, J.; Mewes, H. W.; Mayer, K. F. X.; |
| | Lemcke, K.; Schueller, C.; Quetier, F.; Salanoubat, M. |
| | Submitted to the Protein Sequence Database, November 1999 protein |
| | kinase Xa21; leucine-rich alpha-2-glycoprotein repeat homology; |
| | protein kinase homology |

TABLE 8
Identification of sequences (see also the sequence listing for
specific sequence information).

| SEQ. ID NO | gelcode | Marker name | Sequence reference |
|---|---|---|---|
| 1 | KM3F027R[3] | T14/m43_274.96 | Km3f_tr11-10r |
| 2 | KM3F027R[4] | T14/m44_470.66 | Km3f_tr11-13r |
| 3 | KM3F019L[4] | T12/m49_155.84 | Km3f_tr11-14r |
| 4 | KM3F022R[1] | T12/m51_263.82 | Km3f_tr11-19r |
| 5 | KM3F014L[4] | T12/m39_322.57 | Km3f_tr11-1r |
| 6 | KM3F027L[3] | T14/m48_157.75 | Km3f_tr11-25r |
| 7 | KM3F032R[2] | T13/m62_262.88 | Km3f_tr11-27r |
| 8 | KM3F034L[1] | T13/m55_152.62 | Km3f_tr11-28r |
| 9 | KM3F037L[1] | T13/m76_132.50 | Km3f_tr11-39r |
| 10 | KM3F015L[5] | T13/m40_152.17 | Km3f_tr11-3r |
| 11 | KM3F037L[2] | T13/m77_163.97 | Km3f_tr11-40r |
| 12 | KM3F029L[2] | T14/m92_360.48 | Km3f_tr11-43r |
| 13 | KM3F040R[1] | T12/m81_348.79 | Km3f_tr11-45r |
| 14 | KM3F040R[5] | T12/m85_115.69 | Km3f_tr11-46r |
| 15 | KM3F040R[5] | T12/m85_115.69 | Km3f_tr11-47r |
| 16 | KM3F018R[3] | T11/m43_132.26 | Km3f_tr11-52r |
| 17 | KM3F022L[5] | T12/m60_116.48 | Km3f_tr11-59r |
| 18 | KM3F0L9R[2] | T12/m42_202.77 | Km3f_tr11-5r |
| 19 | KM3F032L[1] | T13/m66_116.54 | Km3f_tr11-62r |
| 20 | KM3F032L[1] | T13/m66_116.54 | Km3f_tr11-63r |
| 21 | KM3F032L[2] | T13/m67_185.99 | Km3f_tr11-64r |
| 22 | KM3F032R[3] | T13/m63_257.70 | Km3f_tr11-65r |
| 23 | KM3F033R[2] | T14/m64_187.95 | Km3f_tr11-66r |
| 24 | KM3F035L[3] | T11/m78_161.83 | Km3f_tr11-67r |
| 25 | KM3F035R[1] | T11/m71_279.09 | Km3f_tr11-69r |
| 26 | KM3F019R[4] | T12/m44_191.57 | Km3f_tr11-6r |
| 27 | KM3F035R[1] | T11/m71_248.89 | Km3f_tr11-70r |
| 28 | KM3F041L[2] | T14/m69_173.70 | Km3f_tr11-76r |
| 29 | KM3F028L[3] | T12/m94_129.36 | Km3f_tr11-78r |
| 30 | KM3F028R[2] | T11/m92_289.36 | Km3f_tr11-81r |
| 31 | KM3F029L[1] | T14/m91_129.50 | Km3f_tr11-83r |
| 32 | KM3F029R[2] | T13/m92_244.73 | Km3f_tr11-84r |
| 33 | KM3F029R[3] | T13/m93_333.06 | Km3f_tr11-85r |
| 34 | KM3F038L[5] | T14/m80_190.22 | Km3f_tr11-88r |
| 35 | KM3F040R[4] | T12/m84_119.28 | Km3f_tr11-90r |
| 36 | KM3F040R[5] | T12/m85_212.74 | Km3f_tr11-92r |
| 37 | KM3F042L[2] | T14/m87_299.12 | Km3f_tr11-94r |
| 38 | KM3F042L[3] | T14/m88_228.31 | Km3f_tr11-95r |
| 39 | KM3F026L[1] | T13/m46_153.93 | Km3f_tr11-9r |
| 40 | KM3F027R[3] | T14/m43_274.96 | km3f_tr11-11R |
| 41 | KM3F027R[3] | T14/m43_274.96 | km3f_tr11-12R |
| 42 | KM3F023L[4] | T11/m59_162.51 | Km3f_tr11-15r |
| 43 | KM3F022R[5] | T12/m55_153.39 | Km3f_tr11-18r |
| 44 | KM3F022R[2] | T12/m52_132.78 | Km3f_tr11-20r |
| 45 | KM3F024R[2] | T13/m52_160.17 | Km3f_tr11-21r |
| 46 | KM3F025L[1] | T14/m57_146.95 | Km3f_tr11-22r |
| 47 | KM3F026L[2] | T13/m47_283.37 | Km3f_tr11-23r |
| 48 | KM3F031R[1] | T12/m61_423.01 | Km3f_tr11-26r |
| 49 | KM3F034R[4] | T13/m59_166.45 | Km3f_tr11-29r |
| 50 | KM3F014R[4] | T12/m34_195.62 | Km3f_tr11-2r |
| 51 | KM3F026L[2] | T13/m47_122.04 | Km3f_tr11-32r |
| 52 | KM3F030R[4] | T11/m64_116.67 | Km3f_tr11-33r |
| 53 | KM3F035R[3] | T11/m73_217.72 | Km3f_tr11-38r |
| 54 | KM3F028L[3] | T12/m94_169.15 | Km3f_tr11-41r |
| 55 | KM3F028R[1] | T11/m91_225.16 | Km3f_tr11-42r |
| 56 | KM3F038L[4] | T14/m79_179.14 | Km3f_tr11-44r |
| 57 | KM3F042L[2] | T14/m87_319.78 | Km3f_tr11-49r |
| 58 | KM3F015R[1] | T13/m31_284.99 | Km3f_tr11-4r |
| 59 | KM3F015R[1] | T13/m31_159.91 | Km3f_tr11-50r |
| 60 | KM3F018R[4] | T11/m44_322.91 | Km3f_tr11-53r |
| 61 | KM3F026R[3] | T13/m43_139.26 | Km3f_tr11-54r |
| 62 | KM3F027R[5] | T14/m45_143.31 | Km3f_tr11-55r |
| 63 | KM3F027R[5] | T14/m45_143.31 | Km3f_tr11-56r |
| 64 | KM3F019L[4] | T12/m49_149.24 | Km3f_tr11-58r |
| 65 | KM3F031L[5] | T12/m70_347.19 | Km3f_tr11-61r |
| 66 | KM3F035R[1] | T11/m71_322.22 | Km3f_tr11-68r |
| 67 | KM3F033R[2] | T14/m64_165.20 | Km3f_tr11-71r |
| 68 | KM3F036R[3] | T12/m73_130.27 | Km3f_tr11-72r |
| 69 | KM3F036R[3] | T12/m73_130.27 | Km3f_tr11-73r |
| 70 | KM3F038L[1] | T14/m76_238.28 | Km3f_tr11-74r |
| 71 | KM3F041L[2] | T14/m69_168.25 | Km3f_tr11-77r |
| 72 | KM3F028L[3] | T12/m94_129.36 | Km3f_tr11-79r |
| 73 | KM3F026L[1] | T13/m46_296.13 | Km3f_tr11-7r |
| 74 | KM3F028L[3] | T12/m94_129.36 | Km3f_tr11-80r |
| 75 | KM3F028R[4] | T11/m94_186.50 | Km3f_tr11-82r |
| 76 | KM3F035L[4] | T11/m79_198.36 | Km3f_tr11-86r |
| 77 | KM3F036L[5] | T12/m80_268.19 | Km3f_tr11-87r |
| 78 | KM3F038L[5] | T14/m80_133.82 | Km3f_tr11-89r |
| 79 | KM3F026L[1] | T13/m46_195.66 | Km3f_tr11-8r |
| 80 | KM3F040R[5] | T12/m85_212.74 | Km3f_tr11-91r |
| 81 | KM3F040R[5] | T12/m85_212.74 | Km3f_tr11-93r |
| 82 | KM3F042R[3] | T14/m83_248.13 | Km3f_tr11-96r |
| 83 | KM3F026L[5] | T13/m50_187.56 | Km3f_tr1114-86r |
| 84 | KM3F033R[2] | T14/m64_134.80 | Km3f_tr1114-89r |
| 85 | KM3F034L[5] | T12/m52_133.18 | Km3f_tr1114-93r |
| 86 | KM3F038R[3] | T14/m73_137.08 | Km3f_tr1114-95r |
| 87 | KM3F030L[4] | T11/m69_226.30 | km3f_tr1114-87R |
| 88 | KM3F035R[3] | T11/m73_237.91 | km3f_tr1114-90R |
| 89 | KM3F018R[3] | T11/m43_132.26 | km3f_tr1114-92R |
| 90 | KM3F035R[1] | T11/m71_279.09 | km3f_tr1114-94R |
| 91 | KM3F107R[2] | T26/m33_173.20 | km3f_t23-A01r |
| 92 | KM3F107R[2] | T26/m35_140.56 | km3f_t23-A02r |
| 93 | KM3F108L[2] | T23/m38_207.27 | km3f_t23-A03r |
| 94 | KM3F108L[2] | T23/m38_207.27 | km3f_t23-A04r |
| 95 | KM3F109R[1] | T24/m31_211.92 | km3f_t23-A05r |
| 96 | KM3F109R[1] | T24/m31_145.60 | km3f_t23-A06r |
| 97 | KM3F109R[2] | T24/m32_132.14 | km3f_t23-A07r |
| 98 | KM3F110L[2] | T25/m39_262.73 | km3f_t23-A08r |
| 99 | KM3F110R[1] | T25/m33_195.61 | km3f_t23-A10r |
| 100 | KM3F110R[1] | T25/m33_109.11 | km3f_t23-A11r |
| 101 | KM3F110R[2] | T25/m34_176.15 | km3f_t23-A12r |
| 102 | KM3F110R[2] | T25/m34_173.23 | km3f_t23-B01r |
| 103 | KM3F110R[2] | T25/m34_114.96 | km3f_t23-B02r |
| 104 | KM3F111R[1] | T23/m42_196.55 | km3f_t23-B03r |
| 105 | KM3F111R[1] | T23/m42_110.23 | km3f_t23-B04r |
| 106 | KM3F112L[2] | T24/m46_248.72 | km3f_t23-B05r |
| 107 | KM3F112L[2] | T24/m47_145.54 | km3f_t23-B06r |
| 108 | KM3F112L[2] | T24/m47_145.54 | km3f_t23-B07r |
| 109 | KM3F112L[2] | T24/m49_114.44 | km3f_t23-B08r |
| 110 | KM3F112R[2] | T24/m43_126.11 | km3f_t23-B09r |
| 111 | KM3F113R[1] | T25/m42_403.04 | km3f_t23-B10r |
| 112 | KM3F113R[1] | T25/m42_403.04 | km3f_t23-B11r |
| 113 | KM3F113R[1] | T25/m42_279.47 | km3f_t23-B12r |
| 114 | KM3F113R[1] | T25/m45_128.72 | km3f_t23-C01r |
| 115 | KM3F114L[1] | T26/m46_283.74 | km3f_t23-C02r |
| 116 | KM3F114L[1] | T26/m46_264.96 | km3f_t23-C03r |
| 117 | KM3F114L[1] | T26/m47_185.12 | km3f_t23-C04r |
| 118 | KM3F114R[1] | T26/m41_112.59 | km3f_t23-C05r |
| 119 | KM3F115R[2] | T26/m38_193.94 | km3f_t23-C06r |
| 120 | KM3F115R[2] | T26/m38_147.62 | km3f_t23-C07r |
| 121 | KM3F115R[4] | T26/m40_148.76 | km3f_t23-C08r |
| 122 | KM3F115R[5] | T26/m36_286.24 | km3f_t23-C09r |
| 123 | KM3F115R[5] | T26/m36_125.56 | km3f_t23-C10r |
| 124 | KM3F1161[3] | T23/m51_190.32 | km3f_t23-C11r |
| 125 | KM3F1161[3] | T23/m58_148.63 | km3f_t23-C12r |
| 126 | KM3F1161[3] | T23/m59_136.04 | km3f_t23-D01r |
| 127 | KM3F116L[3] | T23/m60_129.51 | km3f_t23-D02r |
| 128 | KM3F116R[1] | T23/m51_128.05 | km3f_t23-D03r |
| 129 | KM3F116R[1] | T23/m51_315.68 | km3f_t23-D04r |
| 130 | KM3F117L[3] | T24/m58_208.18 | km3f_t23-D05r |
| 131 | KM3F117L[3] | T24/m59_118.87 | km3f_t23-D06r |
| 132 | KM3F117L[3] | T24/m60_194.55 | km3f_t23-D07r |
| 133 | KM3F117R[1] | T24/m52_127.20 | km3f_t23-D08r |
| 134 | KM3F118L[2] | T25/m58_318.57 | km3f_t23-D09r |
| 135 | KM3F118L[2] | T25/m60_335.11 | km3f_t23-D10r |
| 136 | KM3F118R[2] | T25/m52_131.06 | km3f_t23-D11r |
| 137 | KM3F118R[2] | T25/m53_172.05 | km3f_t23-D12r |
| 138 | KM3F118R[2] | T25/m53_235.21 | km3f_t23-E01r |
| 139 | KM3F118R[2] | T25/m54_161.02 | km3f_t23-E02r |
| 140 | KM3F118R[2] | T25/m55_262.82 | km3f_t23-E03r |
| 141 | KM3F119L[2] | T26/m56_148.92 | km3f_t23-E04r |
| 142 | KM3F119L[2] | T26/m60_121.24 | km3f_t23-E05r |
| 143 | KM3F119L[2] | T23/m66_238.57 | km3f_t23-E06r |
| 144 | KM3F119L[2] | T23/m69_261.94 | km3f_t23-E07r |
| 145 | KM3F121L[1] | T24/m67_190.73 | km3f_t23-E08r |
| 146 | KM3F121L[2] | T24/m70_175.82 | km3f_t23-E09r |
| 147 | KM3F121L[2] | T24/m70_235.78 | km3f_t23-E10r |
| 148 | KM3F121L[2] | T24/m70_235.78 | km3f_t23-E11r |
| 149 | KM3F122L[2] | T25/m68_266.07 | km3f_t23-E12r |
| 150 | KM3F122R[1] | T25/m61_318.48 | km3f_t23-F01r |
| 151 | KM3F122R[1] | T25/m61_114.01 | km3f_t23-F02r |
| 152 | KM3F122R[2] | T25/m63_241.92 | km3f_t23-F03r |
| 153 | KM3F122R[2] | T25/m64_307.66 | km3f_t23-F04r |
| 154 | KM3F122R[2] | T25/m65_379.45 | km3f_t23-F05r |
| 155 | KM3F124R[1] | T23/m71_166.40 | km3f_t23-F06r |
| 156 | KM3F125L[2] | T24/m76_154.52 | km3f_t23-F07r |
| 157 | KM3F125L[2] | T24/m77_258.06 | km3f_t23-F08r |
| 158 | KM3F125L[2] | T24/m78_300.02 | km3f_t23-F09r |
| 159 | KM3F125L[5] | T24/m80_242.25 | km3f_t23-F10r |
| 160 | KM3F125L[5] | T24/m80_144.03 | km3f_t23-F11r |
| 161 | KM3F125L[5] | T24/m80_108.39 | km3f_t23-F12r |
| 162 | KM3F126L[5] | T25/m79_106.38 | km3f_t23-G01r |
| 163 | KM3F126L[5] | T25/m80_111.41 | km3f_t23-G02r |
| 164 | KM3F126R[5] | T25/m71_117.20 | km3f_t23-G03r |
| 165 | KM3F126R[3] | T25/m75_277.11 | km3f_t23-G04r |
| 166 | KM3F126R[3] | T25/m75_243.38 | km3f_t23-G05r |
| 167 | KM3F126R[3] | T25/m75_155.15 | km3f_t23-G06r |
| 168 | KM3F127L[1] | T26/m76_177.10 | km3f_t23-G07r |
| 169 | KM3F127L[3] | T26/m78_203.41 | km3f_t23-G08r |
| 170 | KM3F127L[3] | T26/m78_118.63 | km3f_t23-G09r |
| 171 | KM3F127L[3] | T26/m78_103.18 | km3f_t23-G10r |
| 172 | KM3F127R[3] | T26/m72_149.09 | km3f_t23-G11r |
| 173 | KM3F127R[2] | T26/m73_113.33 | km3f_t23-G12r |
| 174 | KM3F128L[2] | T23/m87_173.78 | km3f_t23-H01r |
| 175 | KM3F128L[2] | T23/m88_190.37 | km3f_t23-H02r |
| 176 | KM3F128R[1] | T23/m83_166.26 | km3f_t23-H03r |
| 177 | KM3F128R[1] | T23/m85_173.13 | km3f_t23-H04r |
| 178 | KM3F129R[1] | T24/m84_132.46 | km3f_t23-H05r |
| 179 | KM3F130L[1] | T25/m86_123.44 | km3f_t23-H06r |
| 180 | KM3F130L[4] | T25/m90_318.54 | km3f_t23-H07r |
| 181 | KM3F130L[4] | T25/m90_129.48 | km3f_t23-H08r |
| 182 | KM3F080L[1] | T21/m36_103.94 | km3f_t23-H10r |
| 183 | KM3F088R[2] | T19/M52_117.14 | km3f_t23-H11r |
| 184 | KM3F078L[1] | T20/m38_192.41 | Km3f_t19-A01r |
| 185 | KM3F078L[1] | T20/m38_131.93 | Km3f_t19-A02r |
| 186 | KM3F078R[1] | T20/m33_135.17 | Km3f_t19-A03r |
| 187 | KM3F078R[1] | T20/m33_99.10 | Km3f_t19-A04r |
| 188 | KM3F078R[1] | T20/m33_94.34 | Km3f_t19-A05r |
| 189 | KM3F078R[1] | T20/m35_109.43 | Km3f_t19-A06r |
| 190 | KM3F080L[1] | T21/m36_106.09 | Km3f_t19-A07r |
| 191 | KM3F080L[1] | T21/m38_236.82 | Km3f_t19-A09r |
| 192 | KM3F080r[1] | T21/m31_362.24 | Km3f_t19-A10r |
| 193 | KM3F080R[2] | T21/m32_252.41 | Km3f_t19-A11r |
| 194 | KM3F080R[2] | T21/m33_343.22 | Km3f_t19-A12r |
| 195 | KM3F080R[4] | T21/m34_117.55 | Km3f_t19-B01r |
| 196 | KM3F080R[4] | T21/m34_107.65 | Km3f_t19-B02r |
| 197 | KM3F080R[4] | T21/m35_139.69 | Km3f_t19-B03r |
| 198 | KM3F081L[4] | T22/m38_441.22 | Km3f_t19-B04r |
| 199 | KM3F081L[2] | T22/m39_153.77 | Km3f_t19-B06r |
| 200 | KM3F082L[1] | T19/m48_117.19 | Km3f_t19-B07r |
| 201 | KM3F082L[1] | T19/m48_113.60 | Km3f_t19-B08r |
| 202 | KM3F082R[1] | T19/m41_442.57 | Km3f_t19-B09r |
| 203 | KM3F082L[1] | T19/m42_280.88 | Km3f_t19-B10r |
| 204 | KM3F082L[1] | T19/m43_156.65 | Km3f_t19-B11r |
| 205 | KM3F082L[1] | T19/m45_153.53 | Km3f_t19-B12r |
| 206 | KM3F083L[1] | T20/m46_284.38 | Km3f_t19-C01r |
| 207 | KM3F083L[2] | T20/m47_321.79 | Km3f_t19-C02r |
| 208 | KM3F083L[2] | T20/m47_99.00 | Km3f_t19-C03r |
| 209 | KM3F083L[2] | T21/m46_249.52 | Km3f_t19-C04r |
| 210 | KM3F084R[1] | T21/m42_338.67 | Km3f_t19-C05r |
| 211 | KM3F084R[1] | T21/m42_220.55 | Km3f_t19-C06r |
| 212 | KM3F084R[1] | T21/m44_119.39 | Km3f_t19-C07r |
| 213 | KM3F085R[3] | T22/m45_186.69 | Km3f_t19-C08r |
| 214 | KM3F085R[3] | T22/m47_132.19 | Km3f_t19-C09r |
| 215 | KM3F087R[1] | T20/m44_254.43 | Km3f_t19-C10r |
| 216 | KM3F087R[1] | T20/m44_216.16 | Km3f_t19-C11r |
| 217 | KM3F087R[2] | T20/m45_183.03 | Km3f_t19-C12r |
| 218 | KM3F088L[2] | T19/m56_117.03 | Km3f_t19-D01r |
| 219 | KM3F088L[2] | T19/m59_197.55 | Km3f_t19-D02r |
| 220 | KM3F088L[2] | T19/m60_105.53 | Km3f_t19-D03r |
| 221 | KM3F088R[2] | T19/m51_111.14 | Km3f_t19-D04r |
| 222 | KM3F088R[2] | T19/m52_450.09 | Km3f_t19-D05r |
| 223 | KM3F088R[2] | T19/m52_195.49 | Km3f_t19-D06r |
| 224 | KM3F088R[3] | T19/m55_315.09 | Km3f_t19-D08r |
| 225 | KM3F089L[2] | T20/m60_139.30 | Km3f_t19-D10r |
| 226 | KM3F090R[1] | T21/m51_258.10 | Km3f_t19-D11r |
| 227 | KM3F090R[1] | T21/m53_164.99 | Km3f_t19-D12r |
| 228 | KM3F090R[1] | T21/m54_180.70 | Km3f_t19-E01r |
| 229 | KM3F091L[4] | T22/m58_246.81 | Km3f_t19-E02r |
| 230 | KM3F091R[4] | T22/m54_213.92 | Km3f_t19-E03r |
| 231 | KM3F092R[1] | T19/m62_142.37 | Km3f_t19-E04r |
| 232 | KM3F092R[1] | T19/m62_116.19 | Km3f_t19-E05r |
| 233 | KM3F092R[1] | T19/m62_105.69 | Km3f_t19-E06r |
| 234 | KM3F093R[1] | T20/m61_434.71 | Km3f_t19-E07r |
| 235 | KM3F093R[1] | T20/m61_178.68 | Km3f_t19-E08r |
| 236 | KM3F094L[1] | T21/m66_455.36 | Km3f_t19-E09r |
| 237 | KM3F094L[1] | T21/m66_237.97 | Km3f_t19-E10r |
| 238 | KM3F094L[1] | T21/m67_156.87 | Km3f_t19-E11r |
| 239 | KM3F094L[1] | T21/m68_273.60 | Km3f_t19-E12r |
| 240 | KM3F094R[1] | T21/m63_115.51 | Km3f_t19-F01r |
| 241 | KM3F094L[1] | T21/m64_149.70 | Km3f_t19-F02r |
| 242 | KM3F094L[1] | T21/m61_334.12 | Km3f_t19-F03r |
| 243 | KM3F095L[1] | T22/m66_142.80 | Km3f_t19-F04r |
| 244 | KM3F095L[1] | T22/m66_113.72 | Km3f_t19-F05r |
| 245 | KM3F095L[1] | T22/m70_269.73 | Km3f_t19-F06r |
| 246 | KM3F095R[1] | T22/m63_178.72 | Km3f_t19-F07r |
| 247 | KM3F095R[1] | T22/m64_307.25 | Km3f_t19-F08r |
| 248 | KM3F095L[1] | T22/m61_163.58 | Km3f_t19-F09r |
| 249 | KM3F095L[1] | T22/m65_216.30 | Km3f_t19-F10r |
| 250 | KM3F096L[1] | T19/m76_328.03 | Km3f_t19-F11r |
| 251 | KM3F096L[2] | T19/m80_253.80 | Km3f_t19-F12r |
| 252 | KM3F096L[2] | T19/m80_117.13 | Km3f_t19-G01r |
| 253 | KM3F096R[1] | T19/m73_128.53 | Km3f_t19-G02r |
| 254 | KM3F096R[1] | T19/m74_148.97 | Km3f_t19-G03r |
| 255 | KM3F096R[1] | T19/m75_150.10 | Km3f_t19-G04r |
| 256 | KM3F097L[3] | T20/m80_380.71 | Km3f_t19-G05r |
| 257 | KM3F097L[3] | T20/m80_131.76 | Km3f_t19-G06r |
| 258 | KM3F097R[3] | T20/m72_133.56 | Km3f_t19-G07r |
| 259 | KM3F098L[1] | T21/m76_182.35 | Km3f_t19-G08r |
| 260 | KM3F098L[1] | T21/m80_375.48 | Km3f_t19-G09r |
| 261 | KM3F098R[1] | T21/m72_151.64 | Km3f_t19-G10r |
| 262 | KM3F098R[1] | T21/m73_206.18 | Km3f_t19-G11r |
| 263 | KM3F099L[1] | T22/m76_263.77 | Km3f_t19-G12r |
| 264 | KM3F099L[3] | T22/m78_433.40 | Km3f_t19-H01r |
| 265 | KM3F099L[3] | T22/m78_243.16 | Km3f_t19-H02r |
| 266 | KM3F099L[3] | T22/m79_99.14 | Km3f_t19-H03r |
| 267 | KM3F099L[5] | T22/m80_170.23 | Km3f_t19-H04r |
| 268 | KM3F099R[5] | T22/m71_159.04 | Km3f_t19-H05r |
| 269 | KM3F099R[5] | T22/m72_197.82 | Km3f_t19-H06r |
| 270 | KM3F100L[2] | T19/M87_315.35 | Km3f_t19-H07r |
| 271 | KM3F100L[2] | T19/M87_205.62 | Km3f_t19-H08r |
| 272 | KM3F100L[1] | T19/M86_128.95 | Km3f_t19-H09r |
| 273 | KM3F100R[2] | T19/m83_265.73 | Km3f_t19-H10r |
| 274 | KM3F100R[3] | T19/m85_168.89 | Km3f_t19-H11r |
| 275 | KM3F100R[3] | T19/m85_265.33 | Km3f_t19-H12r |
| 276 | KM3F017L[2] | T11/m37_355.44 | Km3f_trest-A01r |
| 277 | KM3F017R[4] | T11/m34_115.00 | Km3f_trest-A02r |
| 278 | KM3F014L[1] | T12/m36_133.22 | Km3f_trest-A03r |
| 279 | KM3F014L[4] | T12/m39_131.36 | Km3f_trest-A04r |
| 280 | KM3F014L[5] | T12/m40_295.82 | Km3f_trest-A05r |
| 281 | KM3F014R[1] | T12/m33_141.33 | Km3f_trest-A06r |
| 282 | KM3F014R[3] | T12/m33_289.67 | Km3f_trest-A07r |
| 283 | KM3F014R[1] | T12/m33_141.33 | Km3f_trest-A08r |
| 284 | KM3F022L[1] | T12/m56_197.04 | Km3f_trest-A09r |
| 285 | KM3F022L[1] | T12/m56_414.48 | Km3f_trest-A10r |
| 286 | KM3F025L[3] | T14/m59_298.62 | Km3f_trest-A11r |
| 287 | KM3F032L[1] | T13/m66_159.78 | Km3f_trest-A12r |
| 288 | KM3F038R[1] | T14/m71_226.18 | Km3f_trest-B01r |
| 289 | KM3F038R[2] | T14/m72_127.10 | Km3f_trest-B02r |
| 290 | KM3F040R[1] | T12/m81_381.02 | Km3f_trest-B03r |
| 291 | KM3F050L[2] | T16/m46_246.46 | Km3f_trest-B04r |
| 292 | KM3F052L[1] | T18/m46_300.38 | Km3f_Trest-B05r |
| 293 | KM3F065R[1] | T15/m77_264.72 | Km3f_Trest-B06r |
| 294 | KM3F066R[1] | T16/m78_112.76 | Km3f_Trest-B07r |
| 295 | KM3F072L[2] | T18/m89_379.77 | Km3f_trest-B08r |
| 296 | KM3F101R[1] | T20/m82_255.31 | Km3f_trest-B10r |
| 297 | KM3F101R[1] | T20/m82_255.31 | Km3f_trest-B11r |
| 298 | KM3F101R[1] | T20/m82_253.18 | Km3f_trest-B12r |
| 299 | KM3F101R[1] | T21/m87_159.68 | Km3f_trest-C01r |
| 300 | KM3F101R[1] | T21/m89_355.66 | Km3f_trest-C02r |
| 301 | KM3F102L[3] | T21/m90_169.43 | Km3f_trest-C03r |
| 302 | KM3F102R[1] | T21/m81_130.62 | Km3f_trest-C04r |
| 303 | KM3F102R[1] | T21/m81_106.57 | Km3f_trest-C05r |
| 304 | KM3F102R[2] | T21/m82_294.56 | Km3f_trest-C06r |
| 305 | KM3F102R[2] | T21/m82_110.71 | Km3f_trest-C07r |
| 306 | KM3F103L[1] | T22/m87_209.70 | Km3f_trest-C08r |
| 307 | KM3F103L[1] | T22/m87_99.50 | Km3f_trest-C09r |
| 308 | KM3F103L[1] | T22/m89_116.52 | Km3f_trest-C10r |
| 309 | KM3F103L[1] | T22/m89_116.52 | Km3f_trest-C11r |
| 310 | KM3F103R[3] | T22/m84_176.12 | Km3f_trest-D01r |
| 311 | KM3F104L[3] | T22/m55_160.17 | Km3f_trest-D02r |
| 312 | KM3F105L[3] | T21/m92_131.87 | Km3f_trest-D03r |
| 313 | KM3F105L[3] | T21/m92_131.87 | Km3f_trest-D04r |
| 314 | KM3F106L[2] | T19/m35_286.21 | Km3f_trest-D06r |
| 315 | KM3F106L[2] | T19/m35_277.90 | Km3f_trest-D07r |
| 316 | KM3F106L[2] | T19/m35_256.65 | Km3f_trest-D08r |
| 317 | KM3F115L[1] | T21/m93_183.15 | Km3f_trest-D09r |
| 318 | KM3F115L[1] | T21/m93_180.82 | Km3f_trest-D10r |
| 319 | KM3F109R[3] | T24/m35_190.42 | Km3f_trest-D12r |
| 320 | KM3F111R[2] | T23/m43_242.15 | Km3f_trest-E01r |
| 321 | KM3F112R[2] | T24/m42_265.39 | Km3f_trest-E02r |
| 322 | KM3F123L[1] | T26/m69_396.15 | Km3f_trest-E03r |
| 323 | KM3F124L[1] | T23/m79_379.60 | Km3f_trest-E04r |
| 324 | KM3F124L[1] | T23/m80_206.00 | Km3f_trest-E05r |
| 325 | KM3F124R[1] | T23/m71_390.80 | Km3f_trest-E06r |
| 326 | KM3F130R[1] | T25/m83_230.64 | Km3f_trest-E07r |
| 327 | KM3F130R[1] | T25/m83_230.64 | Km3f_trest-E08r |
| 328 | KM3F130R[1] | T25/m83_178.42 | Km3f_trest-E09r |
| 329 | KM3F130R[1] | T25/m84_186.74 | Km3f_trest-E10r |
| 330 | KM3F130R[1] | T25/m85_171.99 | Km3f_trest-E11r |
| 331 | KM3F131L[1] | T26/m89_121.75 | Km3f_trest-F01r |
| 332 | KM3F131R[1] | T26/m82_152.12 | Km3f_trest-F02r |
| 333 | KM3F132L[1] | T24/m91_118.71 | Km3f_trest-F03r |
| 334 | KM3F132R[1] | T23/m94_140.81 | Km3f_trest-F04r |
| 335 | KM3F133L[1] | T26/m93_478.98 | Km3f_trest-F05r |
| 336 | KM3F134R[1] | T26/m62_208.72 | Km3f_trest-F06r |
| 337 | KM3F042R[5] | T14/m85_156.61 | Km3f_trest-F07r |
| 338 | KM3F043L[2] | T13/m87_152.46 | Km3f_trest-F08r |
| 339 | KM3F043L[3] | T13/m88_228.56 | Km3f_trest-F09r |
| 340 | KM3F043R[1] | T13/m81_122.23 | Km3f_trest-F11r |
| 341 | KM3F043R[3] | T13/m83_171.96 | Km3f_trest-F12r |
| 342 | KM3F050R[2] | T16/m43_123.27 | Km3f_tr15-10r |
| 343 | KM3F053L[2] | T15/m48_129.14 | Km3f_tr15-12r |
| 344 | KM3F062L[1] | T16/m66_127.57 | Km3f_tr15-13r |
| 345 | KM3F055L[1] | T17/m39_103.29 | Km3f_tr15-14r |
| 346 | KM3F055R[1] | T17/m32_138.72 | Km3f_tr15-15r |
| 347 | KM3F055R[1] | T17/m32_137.81 | Km3f_tr15-16r |
| 348 | KM3F056R[1] | T17/m42_284.69 | Km3f_tr15-18r |
| 349 | KM3F056R[1] | T17/m42_259.96 | Km3f_tr15-19r |
| 350 | KM3F046R[1] | T16/m36_104.54 | Km3f_tr15-1r |
| 351 | KM3F056R[1] | T17/m42_139.28 | Km3f_tr15-20r |
| 352 | KM3F057L[3] | T14/m59_171.85 | Km3f_tr15-21r |
| 353 | KM3F057L[3] | T14/m51_141.13 | Km3f_tr15-23r |
| 354 | KM3F058L[1] | T16/m56_241.58 | Km3f_tr15-24r |
| 355 | KM3F058L[1] | T16/m53_152.10 | Km3f_tr15-25r |
| 356 | KM3F058L[1] | T16/m54_194.58 | Km3f_tr15-26r |
| 357 | KM3F059R[1] | T17/m54_262.44 | Km3f_tr15-27r |
| 358 | KM3F060R[2] | T18/m53_268.40 | Km3f_tr15-29r |
| 359 | KM3F046L[2] | T16/m38_177.24 | Km3f_tr15-2r |
| 360 | KM3F060R[2] | T18/m53_199.18 | Km3f_tr15-30r |
| 361 | KM3F061L[1] | T15/m66_201.29 | Km3f_tr15-31r |
| 362 | KM3F061L[1] | T15/m67_253.60 | Km3f_tr15-32r |
| 363 | KM3F061L[1] | T15/m69_111.04 | Km3f_tr15-33r |
| 364 | KM3F061L[1] | T15/m61_167.19 | Km3f_tr15-34r |
| 365 | KM3F061L[1] | T15/m61_167.19 | Km3f_tr15-35r |
| 366 | KM3F061L[1] | T15/m62_225.54 | Km3f_tr15-36r |
| 367 | KM3F061L[1] | T15/m65_224.11 | Km3f_tr15-37r |
| 368 | KM3F062L[1] | T16/m66_130.52 | Km3f_tr15-38r |
| 369 | KM3F062L[1] | T16/m66_130.52 | Km3f_tr15-39r |
| 370 | KM3F046R[1] | T16/m31_213.31 | Km3f_tr15-3r |
| 371 | KM3F062L[1] | T16/m66_129.60 | Km3f_tr15-40r |
| 372 | KM3F062L[1] | T16/m66_126.64 | Km3f_tr15-41r |
| 373 | KM3F062L[1] | T16/m66_126.64 | Km3f_tr15-42r |
| 374 | KM3F062L[2] | T16/m61_105.18 | Km3f_tr15-43r |
| 375 | KM3F062R[2] | T16/m63_206.19 | Km3f_tr15-44r |
| 376 | KM3F062R[2] | T16/m63_119.24 | Km3f_tr15-45r |
| 377 | KM3F063R[1] | T17/m64_294.32 | Km3f_tr15-46r |
| 378 | KM3F063R[1] | T17/m64_211.16 | Km3f_tr15-47r |
| 379 | KM3F064R[1] | T18/m61_210.04 | Km3f_tr15-49r |
| 380 | KM3F048L[1] | T18/m38_152.26 | Km3f_tr15-4r |
| 381 | KM3F064R[1] | T18/m61_181.90 | Km3f_tr15-50r |
| 382 | KM3F064R[1] | T18/m62_180.75 | Km3f_tr15-51r |
| 383 | KM3F065R[1] | T15/m73_202.18 | Km3f_tr15-52r |
| 384 | KM3F066R[1] | T16/m80_119.58 | Km3f_tr15-54r |
| 385 | KM3F066R[1] | T16/m73_131.08 | Km3f_tr15-55r |
| 386 | KM3F066R[1] | T16/m74_315.97 | Km3f_tr15-56r |
| 387 | KM3F066R[3] | T16/m75_239.27 | Km3f_tr15-57r |
| 388 | KM3F068R[3] | T18/m76_169.66 | Km3f_tr15-58r |
| 389 | KM3F048L[2] | T18/m39_108.03 | Km3f_tr15-5r |
| 390 | KM3F068R[2] | T18/m78_111.26 | Km3f_tr15-60r |
| 391 | KM3F069R[2] | T15/m84_107.06 | Km3f_tr15-61r |
| 392 | KM3F070L[1] | T16/m87_118.01 | Km3f_tr15-62r |
| 393 | KM3F070L[1] | T16/m89_219.49 | Km3f_tr15-63r |
| 394 | KM3F070R[2] | T16/m82_249.46 | Km3f_tr15-64r |
| 395 | KM3F070R[2] | T16/m82_158.21 | Km3f_tr15-65r |
| 396 | KM3F070R[2] | T16/m82_158.21 | Km3f_tr15-66r |
| 397 | KM3F070R[3] | T16/m83_304.26 | Km3f_tr15-67r |
| 398 | KM3F070R[3] | T16/m84_170.22 | Km3f_tr15-68r |
| 399 | KM3F072L[1] | T18/m87_150.02 | Km3f_tr15-69r |
| 400 | KM3F048L[2] | T18/m35_173.35 | Km3f_tr15-6r |
| 401 | KM3F072L[1] | T18/m87_132.45 | Km3f_tr15-70r |
| 402 | KM3F072L[2] | T18/m89_106.44 | Km3f_tr15-71r |
| 403 | KM3F072L[4] | T18/m88_433.55 | Km3f_tr15-72r |
| 404 | KM3F072L[4] | T18/m81_372.94 | Km3f_tr15-73r |
| 405 | KM3F072R[2] | T18/m82_258.08 | Km3f_tr15-74r |
| 406 | KM3F072R[2] | T18/m82_140.67 | Km3f_tr15-75r |
| 407 | KM3F072R[2] | T18/m84_243.08 | Km3f_tr15-76r |
| 408 | KM3F073L[1] | T17/m87_196.39 | Km3f_tr15-77r |
| 409 | KM3F074L[2] | T17/m85_105.75 | Km3f_tr15-78r |
| 410 | KM3F074L[2] | T17/m85_96.86 | Km3f_tr15-79r |
| 411 | KM3F050L[2] | T16/m47_107.03 | Km3f_tr15-7r |
| 412 | KM3F074L[2] | T15/m91_288.03 | Km3f_tr15-80r |
| 413 | KM3F074R[2] | T15/m92_115.90 | Km3f_tr15-81r |
| 414 | KM3F074R[2] | T15/m92_115.90 | Km3f_tr15-82r |
| 415 | KM3F075L[2] | T18/m93_178.23 | Km3f_tr15-83r |
| 416 | KM3F075L[2] | T18/m93_149.84 | Km3f_tr15-84r |
| 417 | KM3F075L[2] | T18/m93_181.25 | Km3f_tr15-85r |
| 418 | KM3F055L[1] | T17/m39_103.29 | Km3f_tr15-88r |
| 419 | KM3F050R[2] | T16/m43_224.52 | Km3f_tr15-8r |
| 420 | KM3F050R[2] | T16/m43_153.87 | Km3f_tr15-9r |

1-12. (canceled)

13. A nucleic acid molecule that hybridizes under stringent conditions with at least a part of a nucleotide sequence as defined in one of SEQ ID NO 1 to SEQ ID NO 420.

14. A nucleic acid molecule according to claim 13, having a length of 10-50 nucleotides.

15. A collection of at least two different nucleic acids molecules of claim 13.

16. A collection according to claim 15 in the form of an array, wherein the nucleic acid molecules are immobilized to a solid support whereby each different nucleic acid molecule is attached to a distinct part of the solid support as an array of nucleic acid molecules.

17. A method for analyzing a plant genome comprising testing the genome for the presence or absence of nucleic acid sequences that hybridize with the nucleic acid molecule of claim 13.

18. A method for determining whether a plant can manifest a property related to disease resistance, comprising testing the plant for the presence or absence of a nucleic acid molecule according to claim 13.

19. A method for cloning a full-length ant gene or plant cDNA molecule, comprising:
(a) screening a genomic- or cDNA-library for clones comprising a partial or full-length copy of the plant gene or cDNA molecule with a hybridization probe comprising the nucleic acid molecule of claim 13; or,
(b) amplifying a partial or full-length genomic or cDNA-fragment of the plant gene or cDNA molecule from a primer comprising the nucleic acid molecule of claim 13; and,
when said copy or said fragment is partial, optionally assembling partial copies or partial fragments obtained in (a) or (b) or both (a) and (b) into a full-length copy of the plant gene or plant cDNA molecule.

20-21. (canceled)

22. A nucleic acid molecule according to claim 13 comprising a functional coding sequence for a plant polypeptide, which coding sequence:
(a) is part of a plant transcript that hybridizes under stringent conditions with at least a part of one of said nucleotide sequences; or
(b) encodes a polypeptide that has at least 40% amino aid sequence identity with a polypeptide encoded by the coding sequence of (a).

23. A nucleic acid construct that comprises
(i) the nucleic acid molecule of claim 22, and
(ii) an expression regulatory sequence operably linked to the coding sequence that is capable of directing expression of the coding sequence in a suitable host cell.

24. A host cell comprising a nucleic acid construct according to claim 23.

25. A host cell according to claim 24, that is a plant cell.

26. A host cell according to claim 24, that is an Agrobacterium cell.

27. A plant comprising a host cell according to claim 24.

28. Cultivation material for a plant, that comprises a host cell according to claim 24.

29. Plant material obtained from a plant according to claim 27.

30. A polypeptide encoded by the nucleic acid molecule of claim 22.

31. A method for producing a polypeptide comprising growing a host cell according to claim 24, under conditions conducive to the expression of the polypeptide, and optionally recovering the polypeptide,
wherein the coding sequence for said polypeptide:
(i) is part of a plant transcript that comprises a nucleotide sequence that hybridizes under stringent conditions with at least a part of a nucleotide sequence as defined in one of SEQ ID NO 1 to SEQ ID NO 420; or.
(ii) encodes a polypeptide that has at least 40% amino acid identity with the polypeptide encoded by the coding sequence of (i).

32-35. (canceled)


SEQUENCE LISTING (420 sequences)

SEQ ID NO: 1 - DNA, 271 bp, Lycopersicon esculentum
ttaaatatacaatttggaaagtgggagattacaatgaaactctaggcggggtattattggagactggaggaagcatagggcaaagagatagcagctatttcaagattgttccatcaaaacttggttacaacttagtcctttgcgatcctactcctattttctgtccattttgccgtaagggtcagttatgtgtaaatgtgggtgtagttttccaagatggaagaaggcgtttggctcttactaaggatcagcctcttgatgtattattcga

SEQ ID NO: 2 - DNA, 125 bp, Lycopersicon esculentum
ttaaaggagctgctcctctggtgactacctgaatatggagtgtgtgtagcagtagcaccactgacaaacaattttgtggataacataagatctactagtagtcctcactgcccacatctcttcga

SEQ ID NO: 3 - DNA, 83 bp, Lycopersicon esculentum
tcgatgcaatatgttacacccacgattcactacctaattagtgtaaattctaggttgtggagtgatggttgctacgcttttaa

SEQ ID NO: 4 - DNA, 149 bp, Lycopersicon esculentum
tcgatggagtattttggtaatataggttggaagactaggtaaattacggctgctgcgatgcctatgagtacgattagtaagaaaaataggcaacatgtccaacataggcatcttacgcagcagcctcttttcttttttggtgattttaa

SEQ ID NO: 5 - DNA, 146 bp, Lycopersicon esculentum
ttaaaattagagaataaaaaaactcagtctaacgctacaggatcccgaaagctctgttccatgagcttcaaaaattcagaaaattcaatttttccatctctgttttcatcaaaggccataatcatcttcttgcaatgctccatcga

SEQ ID NO: 6 - DNA, 88 bp, Lycopersicon esculentum
tcgatgaagctctctttcaaatgatgttgtactattttcttgtgaataccatgtagcttctgaaaagctctcaagcctgcaattttaa

SEQ ID NO: 7 - DNA, 169 bp, Lycopersicon esculentum
tcgatatcactttcttcaaacttgttgtttgatcctactgcttttgatatctctgttttcacttcagccattgcttgtggatggcgcaaaagctctgttagtgcccattcaacgctactgcttgtcgtctctgtaccagctagaaacaattccagtatgaatactttaa

SEQ ID NO: 8 - DNA, 90 bp, Lycopersicon esculentum
ttaaagtgggcaccacagttggaaatattggggcatttatctacggggtggatttatgaactcactgtgtgatggaattcatgtattcga

SEQ ID NO: 9 - DNA, 174 bp, Lycopersicon esculentum
tcgatcattcatcccgttacgtaaagccttttcgagctggcgcctctgtcaaatacttttaatccctgacatgttatggtctcgggccgaaactgcagatgcgatgctttaagtaaatagcgaaatctgcagcgtacgaaaacagttgctccgaagagggattcccgaatttaa

SEQ ID NO: 10 - DNA, 118 bp, Lycopersicon esculentum
tcgatccaaatagagcatgaagtctcacacagtaatactttgccataaagatctttgctttgtccttatggttcttctccacatagctaactacgagttttcccactggtattgttaa

SEQ ID NO: 11 - DNA, 117 bp, Lycopersicon esculentum
ttaacaatggtccagattggatgggtttgttctcagtaattcctccatctccattgtgatgggattcaattacagtgtcaacaggttccatattcatcttgggatcagtgtgatcga

SEQ ID NO: 12 - DNA, 88 bp, Lycopersicon esculentum
tcgatcaacaagtccccggcatggaacacacatgtataaatattggtgttgcccaaccacattccattccccatggtcctgctgttaa

SEQ ID NO: 13 - DNA, 137 bp, Lycopersicon esculentum
ttaagtgatttccggtcgtccaacaatgtttcaaccggtgaaacacctatccaacattattcaagccaatgaaatgcatgtccaaaacaatgtctaacaaaactaaaatgatcataactttttactccgaacttcga

SEQ ID NO: 14 - DNA, 100 bp, Lycopersicon esculentum
tcgatccacaacatcaacacaacaataaaggggccaaaggatgattcaaagatttactagagttcttacttcttttttatgaatagggagtaatatttaa

SEQ ID NO: 15 - DNA, 373 bp, Lycopersicon esculentum
tcgatgaagtttatggaagtgaatcctccattattggaagcctgaaatctgtcaacgatttcaacccagttgaagttccatcaaatcggcccgttatctttgataataacatgcttgaggacaatttgcagcagccattggctgaaagcgataatgccacagaaaaccttgttagtgagaacggagatgagccaatggactcgggagaaggtgatggagcccagacaaggggcaaaagtgctagtagtagacaaaatgagaagttatacggtgaggaaggcatgttgaacactaaacagaagaaagctgagaagaaaaggaggaagaaagacaagccctcaactgccattgatatggacggtgatgactttaa

SEQ ID NO: 16 - DNA, 221 bp, Lycopersicon esculentum
tcgatggaatgatatggagtggatgtgcaaatggattacttgtccaatgggatagaaatggcaatcgcttgcaagatttccagtatcacacattctccgttcaatgtttatgcacatatgggtcacggatttgggcaggttatgctagtggttatattcaggtattggatcttagtggtaatctacttggaggatggataggtcatagtagccccgtacta

SEQ ID NO: 17 - DNA, 255 bp, Lycopersicon esculentum
ttaaatgtgccaccaccatccaccgatagagaaccacactaagacaaccatcaaacaatctcatccccacttcgctccggtgaaaccaccacaaacaacaaccaaacaactcatttctttctcttctttctccaacacaccagcaataagaagccacgaactccgaagaatcggcgagctcaaagaagcgaatcagatttgaccacaaatcaaagcaaatcagcacacaacaacgaagtaaactaacaccatcga

SEQ ID NO: 18 - DNA, 104 bp, Lycopersicon esculentum
  misc_feature (39)..(): n can be a, c, g, or t
tcgatggcttagccttcatgggaaatcgcttcactgganaatatagcctcatatcctctttggagaacactcacatcacttgagtacttggatatttcatttaa

SEQ ID NO: 19 - DNA, 254 bp, Lycopersicon esculentum
tcgattgaagttgtgttgaacatgcaagtcttcacccttgctttgtcaccgggcgaacacgttgtagccggggcgggagggggatggctatggctatgtggtggaggtggtggtgatcgtggtggtggaggtggtgggcatggttgtggttgtggtcgtggtcgtggtcgtggtcgtggagggcatggatatggtcgtggacagggacatggccatggtaaataacatggaatatcccatggccataaatttaa

SEQ ID NO: 20 - DNA, 237 bp, Lycopersicon esculentum
tcgattcacacatagtattttcgttctgtttatcatgaatctcaactgttttcaattgaaattttcaggagtatatttgccagatttgtgcagcaaacagaggagcagcccacaccgcgcaatgattgagttattgagcagtggagctgctgctgctactgatgcttctgctccgatttcttctgaagatattattgttggtgctcctgatatttcccctgcttctttctaatttaa

SEQ ID NO: 21 - DNA, 162 bp, Lycopersicon esculentum
ttaacaagtgtatcaagcatggcaatcctactagaatatggcttttttgatgcatatgctggagaatcaaaggatcattcacccgactggtgaaattctacacatgggaacattgtttccatgaaactctgcctctttctcccatcacgacatgccaatcga

SEQ ID NO: 22 - DNA, 88 bp, Lycopersicon esculentum
tcgatttaccagttcccggagtgtccataaagcaagtaacctcttttccatgctttaccaatcctgctatagtactctttccctttaa

SEQ ID NO: 23 - DNA, 166 bp, Lycopersicon esculentum
ttaaactccttgacctaacattttcatgaaataaatcagacaaacaattcccaatattatctggtgaaaattttactaacacctccaaatctttttccccactcaaaactaccatatagtactcataaaaatccctataaaaaggcaccaatttccttgcaatcga

SEQ ID NO: 24 - DNA, 104 bp, Lycopersicon esculentum
ttaattattcaataattctatattgatgtatttgttatttgactttgattacaattttgttggagcgtgcctttgattttttggtttggttttgcactattcga

SEQ ID NO: 25 - DNA, 121 bp, Lycopersicon esculentum
tcgattacaaacacgattttgtgggatacgttgcagtcattcttgttggaatatcagttgcttttctcttcatttttgcttactcaatcaaagcattcaacttccaaaaaagatagtttaa

SEQ ID NO: 26 - DNA, 104 bp, Lycopersicon esculentum
ttaaagccacctagatctccaagtccaagaccgagtggtttgaaatcctcagcaactgcggaagtaacatcagcagttactggaggcaacgatgtggcaatcga

SEQ ID NO: 27 - DNA, 239 bp, Lycopersicon esculentum
ttaaaccagagaagcgtcatggagtatacttgagaaaaatgttcctccttctgcatctgattctcaaacttatagcgtctttggtggtgcaaacttcacattcatactaaacagattcataaatatatcattggttacaaagtcaggtaaacctaccaaccaaattgtagagaaccgtgtacactatgttacaatgagctggttgctaagggagttcaactgctatcctcctcaatcga

SEQ ID NO: 28 - DNA, 76 bp, Lycopersicon esculentum
ttaaaccacactaagcatataaaaacatgatcccttacctagactataagtctatgtaaaatatcacatgaatcga

SEQ ID NO: 29 - DNA, 164 bp, Lycopersicon esculentum
ttaacgccaatgggtgcagtatttgcaatgtacttgaaggttttcaaagcagcaaatccagcttggttttatatacatgttgcttgtcaaacttctgcatatattgttggtgttgctggatggggaactggtctcaaacttggcagtgattctaccggtatcga

SEQ ID NO: 30 - DNA, 123 bp, Lycopersicon esculentum
ttaacgtcatcatacaacatgtagaactgatccaagtacttctgttgcatccgcattctagccttcagcagcttggattcaacaactgaaaaggattacaatggctacgaattagtgtatcga

SEQ ID NO: 31 - DNA, 110 bp, Lycopersicon esculentum
tcgatagccttatacccctccatcaaatcatcatcctgggccatgtcaaggaaagattgaagctctagagctctgcggtaatacatcatccctctcacagtcctagttaa

SEQ ID NO: 32 - DNA, 100 bp, Lycopersicon esculentum
ttaacacttgggctgatatcatcaaccgtgctaaccttggtatggaagttatgcatgaacgtaatgctcataacttccctctagacctagctgctatcga

SEQ ID NO: 33 - DNA, 101 bp, Lycopersicon esculentum
ttaaccactacatgagtatactatttgtggaactatgacactgtctgaacaaacaacagaactagggctactactataaaagaaatctaaatctttatcga

SEQ ID NO: 34 - DNA, 288 bp, Lycopersicon esculentum
tcgatatggccctgttcttctcctccaattcggttcccggaaagttcttctcgtttcatcgcccgccggagcggaagagtgtttcacgaagaacgacgtcgttttcgcaaatcgccctcatttgatggccggaaaacatctaggttataattttacatcgttggcttggagttcttatggagatcattggagaaatctacggagaattacttccgttgagatgttttcaactcatcgtcttcaaatgcttcacggaattcgtgtggatgacgtgaaatctatggttaa

SEQ ID NO: 35 - DNA, 320 bp, Lycopersicon esculentum
ttaatagaatgggagaattgttagagcttccactttctacacaaaaccatctgaaaagatcagatgatgcctgcgaactctcacccattgtaggtccaaaaatgggatcctttgctttctccccagtcccagaaactggataagctttcacaaattttcctttgttgagccattcatcggccggacttggctcgtctgaatacttccctccactaaaagaaaagccgtccccaactgaaacaggagcatccagcaaataatcctccttatcatcactactggatcggcaattgaatcgcaaaatccttgcctgagttcga

SEQ ID NO: 36 - DNA, 181 bp, Lycopersicon esculentum
tcgatctagtacgaacaccaagagaagaagactccataacatggccactttttctcaagtattttcccatgaatgttttgtaatggacaacagaggagggaaaataaaaagggatgagtttttaggtaaagttagggttgaaacgggatacttatgactcaacactcaacagtaacgttaa

SEQ ID NO: 37 - DNA, 92 bp, Lycopersicon esculentum
ttaactagatagtctacctaaaggtgtagacccgtcatacagacacaaacctctccaacatgatgttcgcaagatagtaggaatgggatcga

SEQ ID NO: 38 - DNA, 167 bp, Lycopersicon esculentum
ttaactctttgtacaaagtaaagatctctattgaaatctttcatgttttgaattatccttcatgatgaaaacctcaaacctacaataccagaaaatccaaatatcctaaaaccacttgagcagccagcaatatacaaaagctatcgtcgtgtatgtgttttgatcga

SEQ ID NO: 39 - DNA, 101 bp, Lycopersicon esculentum
tcgatcccaactggtacataagggtggcagctcaggggagaaagcctagcatgggccaatcccaaattgtgtaattatgaggaccgcaacccacgggttaa

SEQ ID NO: 40 - DNA, 101 bp, Lycopersicon esculentum; identical to SEQ ID NO: 39

SEQ ID NO: 41 - DNA, 306 bp, Lycopersicon esculentum
tcgatgatccaggagtcatcaaaggccaaggtacaattggaacagagataaaccgtcaactgaaggacattcacgctgtgtttatacctgttggtggtgggggtttgatagctggtgttgctacctttctcaaacaaattgccccaaatacaaaaattattggagttgagccatatggtgcagcttcaatgacattgtcattgcgtgaaggacatagagtgaaattatcaaatgttgatacttttgctgacggtgtagctgttgcactagttggtgaatacacttttgcaaaatgccaagagttaa

SEQ ID NO: 42 - DNA, 134 bp, Lycopersicon esculentum
tcgatgcaaagcgaagaaccttaccagtggcttgacatgccgcgaatcctcttgaaagagaggggtgccttcgggaacgcggacacaggtggtgcatggctgtcgtcagctcgtgccgtaaggtgtcgggttaa

SEQ ID NO: 43 - DNA, 161 bp, Lycopersicon esculentum
ttaaccgactgtttgtagggggtgacagtgctggaggcaatatagtttataatatgatcatgagagcggggagagaaaaattgatcggagatgtgaaaattttaggtgcaatacttggatttccttatttgatgataccatcggtacgcagtctacatcga

SEQ ID NO: 44 - DNA, 207 bp, Lycopersicon esculentum
ttaaccgaacagtgcacacccctaatttcatccttactaggagtatttgaaaagatttataaagatgataacttaccctacaaggggtgctaacatcacaataagcttcaagagtgatcaagacttgattgtccaaatcctcagcatacttataacaagggcaactagttccaagtatgctttggaatgttggacaacactcatcga

SEQ ID NO: 45 - DNA, 134 bp, Lycopersicon esculentum
ttaacctctatacacttcaaagcacaaaataaatgcagcgaaaggagccgagttgatagaagtttgcgtatctttgttgatgaaattcaccatctacttggcagaataccagtttttagcacatttagcatcga

SEQ ID NO: 46 - DNA, 83 bp, Lycopersicon esculentum
ttaatcgggcatgtctggtttaggattggattcggatgtgtaggtctcaggaaaattatccaatcctaagggtatgggttcga

SEQ ID NO: 47 - DNA, 235 bp, Lycopersicon esculentum
ttaacgatgacttgcatgtaagacaatagttccaagccagttggttctttgcacttcaattttttatatatgatgcttcctgacgatagtagtcgtgctctaatcccttccaacacgtatgcacccaatcgttccatcggttccccagacacagacacccgttgctctaaagcactcataagagcttcagcagtagagatatcagcatctgacactgcttcagcacaggcatcga

SEQ ID NO: 48 - DNA, 129 bp, Lycopersicon esculentum
tcgattggaatttgtttgcatgattgtccgctatctgtgtcgctgtcgtggagaagagaggagacgaaagcagcgctatttcaccttgtttgttgttattttgattatgatttactatgattgcgttaa

SEQ ID NO: 49 - DNA, 94 bp, Lycopersicon esculentum
ttaactctttgggcggtttcgtaatgttcctcaccaacgatccgaggttgaagcatggttgaccttaaatcttagtgtactgacccctaatcga

SEQ ID NO: 50 - DNA, 207 bp, Lycopersicon esculentum
ttaagatatcactgtctagaaagattgtttcgggttatcggatcttttacgaaaagtacagggaatcactgagaagtggcggagtgaagtcagttgtcagatttgcccctgacgatttgcaaaattacttatcggatttattctatggcactgggttgtcggagcaagcagcgacggcgtacggctctgcttccccgtcggtatcga

SEQ ID NO: 51 - DNA, 235 bp, Lycopersicon esculentum
tcgatacagccgaaaaattcctcaagacttatgatcatatctttgctacaaggcctcataacgaggccgctaagtatttgtcatatggtcataagaatttggtgtttggaacatatggaccatattggcgcaacatgcgcaaattggtcactctagaccttttgacacatcaaaagatcaattcatttcaatccgtcagaacagaacaagttgatcttatgatccaatcgcttaa

SEQ ID NO: 52 - DNA, 165 bp, Lycopersicon esculentum
tcgatcctaagggtcgggggaaccccgacagatagcgcgtttcgcgcgtactccgaaagggaatcgggttaaaattcctgaaccgggacgtggcggttgacggcaacgttaggaagtccggagacgtcggcgggagcctcgggaagagttatcttttctgcttaa

SEQ ID NO: 53 - DNA, 149 bp, Lycopersicon esculentum
tcgatccaaagttcaagaagatgacatctggactttagtcagcattctgagtaaatgcgctcggatgtgaattccgtcagcgtggttagctcagaaatatcaagtttggtctagattgaccctttgttcggggctcacaattagcttaa

SEQ ID NO: 54 - DNA, 209 bp, Lycopersicon esculentum
tcgatcaaactctctactttcgcactaatattagtgaaagttgttgcagatcccttcaaatgtagcagccgaattgaactatccatgctattgaagaatttttccaacatttcatacttttccggcagctttaccgagttcacatgcttcttccgttcagaaaccggcgatccagctaccaatggagatttcacttcaccgtagcttaa

SEQ ID NO: 55 - DNA, 210 bp, Lycopersicon esculentum
tcgatcctgattcagtgtatgaaacatggcatattttgtattcgggagctactaatatggattcggccacttatattcctgatactttggagacagaaacaagacaagacttgcagttcactgtagataaaccttctaacctgtctcagcatggggtcaaacagaatgatggattggttgaagtattgcttgatcaatctattagcttaa

SEQ ID NO: 56 - DNA, 240 bp, Lycopersicon esculentum
ttaagcctaaaaacaagcggaaaaagaagaaatcgtctaatactgccgcagtttagagaatgcatgtgtgatctatcttcttgaggtacgtagttgattatgattgaaattactctgcacggtaatagtaaattctgtgactcgttgcacagttggagaggcctcatgaagttccagatgcaagcagttgtatcattgtttctcaaattgaaggacatgaattgaaaccattgccatcga

SEQ ID NO: 57 - DNA, 83 bp, Lycopersicon esculentum
ttaatcgcgtcaagatctggattctgcaggcgggaagtgatcaagaccacgtcatctccctcctccctaatcaagtgggtcga

SEQ ID NO: 58 - DNA, 293 bp, Lycopersicon esculentum
tcgatggatcagaagaaacggttagccctagaaagtaaaggtgatctgttacctgacaaaactgctggaaggaaagaagcgggagttccttcccaggacaatgctcagaaggatctaaattctcttccagccaaatcaattgatgatgagtacagacagagggccagcagtgatatagatgtccgtaggggtgattcagatgagcttatggacaaaagaactgtcaagaaagaggaagatggcacttttttgaagcctaaatctgatgccaaatcagctgatgcaacagttaa

SEQ ID NO: 59 - DNA, 87 bp, Lycopersicon esculentum
ttaactgaatatggaacgtctgatctggacgtaagagtcagacattgcttttggcaatgtggatactactattattgcgcccatcga

SEQ ID NO: 60 - DNA, 232 bp, Lycopersicon esculentum
tcgatgaaaagcttcgtgttgaaatgtctccacctttggccttgttgatttcagaaactctatatgcttgcttactcttgagcaacatggtccacggacagccctatgagaatattccagagatcacgtagaaaaggaacagtgggtttctgccgacggacccggtcaagcttcacatcagctatgaactgcaagtggcagctcgatgaactggggatttgcgttgctttaa

SEQ ID NO: 61 - DNA, 280 bp, Lycopersicon esculentum
tcgatgtgataaacctataaggctcatccttcttagcccaatattgttgttgatccttctttgatgttacctcttgcaagaaatctgccacgcctttcctctcggggcatttgaatcccatggattcaaagaagtcaagaacatcttcccgaggaccctgatagacaatttgtgcatctgacagcagaattatgtcatcaaacaagttgtaagtctcgggggctggctgtaacagagatattacagcagttcccttcaagagctgcaccgattgtcttaa

SEQ ID NO: 62 - DNA, 375 bp, Lycopersicon esculentum
tcgatgtgtgaatatgatagcacgtttattcaatgcgcctttagaagaggaagaagcagcaattggtgttggcacagtggggtcatcagaggccataatgttagcgggcctagccttcaagaggaactggcaaaacaaacgcaaagctgagggaaagccttatgataagcccaatattgttactggcgctaatgttcaggtgtgttgggagaaattttgcaacttactttgaagtgtgcaattcgagcactctagctcctcgaggacgggtgagtacgacgatgacgccaaaccaccagccaccgccagaaaaaaggtcaaacgcgacaacaaccaaaggcgtggacaagcaaaaacgagggtccaaacccttaa

SEQ ID NO: 63 - DNA, 102 bp, Lycopersicon esculentum
  misc_feature (98)..(): n can be a, c, g, or t
tcgatcccaatttgtatataagggcagcagctcaggggcaaaagcctaagatgggcaaatcccaatttgaggaattatgaggacagcatcccaggggnttaa

SEQ ID NO: 64 - DNA, 117 bp, Lycopersicon esculentum
tcgatcacataggcatgtttctggtcctgcccgcagcttgattgatactcttcaagctgctggtttaggcgctactggtgttatgtctgtcctaataaagcaatctggtggacttaa

SEQ ID NO: 65 - DNA, 231 bp, Lycopersicon esculentum
ttaagtgacagtatcttatcattgatagcttgcattgtggacgtaatgctcatggagccatcatcttcagggcgaccaagcttgcttatatcctgataaaacctaaataccagccctggagaatggtgcagtatatgataatactgctgcacaaatgcatttcccacaacttgagcagatacaggtagttgagccgccgccgccgccactgccgccgccatcgccgatcga

SEQ ID NO: 66 - DNA, 273 bp, Lycopersicon esculentum
ttaagtttgctcacatcattgttttctttcttcttgcgacttcctttgaaactctcatggcacgaaaagaaattgatgggccagaagtcatagaacttctaaaggaatttgactctaacttgatgtgcgaaggacaacaaatgtggcccagaacttattggtgtaccaacaaagcttgctaaggaaataattgagaaggaaaatccatccataactaatattccaatattattgagtggttctccaattacattagattatctatgtgatcga

SEQ ID NO: 67 - DNA, 217 bp, Lycopersicon esculentum
ttaataccactaaacatgtcagttgatttgtttcgtgtagtgtcatatgcacacatcaggtcaagtaaaaccttcatatgcctggtgatatatgttacctgcaccgagtttggaaaatttagatcttgaaacttattgtaagtgcgagtattgacatgtgcaggttgatgttccctcttgctttgttggtctggtattggaattggagaatgatcga

SEQ ID NO: 68 - DNA, 104 bp, Lycopersicon esculentum
ttaaataaatacatgtggtggcgtgatttctccaaacaaaatgtttctctatgagcaaggttttctttcaatttcagcattgaaccagaatgacatggtttcga

SEQ ID NO: 69 - DNA, 117 bp, Lycopersicon esculentum
tcgatcccattatggttctttcagtcctatttcaccatttccttcaatgacatgtgacttgaggggacctaatgcaatggcagaggatgaaaaggcggaaaccttggtgggtattaa

SEQ ID NO: 70 - DNA, 82 bp, Lycopersicon esculentum
ttaatacgcgatagaaaacaaaatatagcgcgcaaactaggataaattatcgcgcgcggtgtcatctatgttactagatcga

SEQ ID NO: 71 - DNA, 80 bp, Lycopersicon esculentum
tcgatggagctttcaggacctagatgatgacatctgcttctattcttgccttgaaaatctgcctctgcctgtattattaa

SEQ ID NO: 72 - DNA, 85 bp, Lycopersicon esculentum
tcgatgagtggacgagagaatgaaaagcaccaccagtaccagcagctctgtttctgtataatacattgttatctatgggtattaa

SEQ ID NO: 73 - DNA, 89 bp, Lycopersicon esculentum
tcgatgctagaggtgtcgaaaagttaccacagggataactggcttgtggcagccaagcgttcatagcgacgttgctttttgatccttaa

SEQ ID NO: 74 - DNA, 253 bp, Lycopersicon esculentum
ttaagtacttgaagcacaagattcatgtgattgatgacaagaatttggtaacaaaatattcacttattgaaggtgatgttcttggtgacaaattggaatcaattgcctatgatgtcaaatttgaagctgctggagatggaggttgtgtttgcaagacaacaactgagtatcacacaaagggtgatcatgttgttagtgaagaagaacacaatgtaggcaaagggaaagccattgacctattcaaggccatcga

SEQ ID NO: 75 - DNA, 216 bp, Lycopersicon esculentum
ttaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcga

SEQ ID NO: 76 - DNA, 127 bp, Lycopersicon esculentum
tcgatgcagacacaaatataaacccagatatgtggaaataggaacacaataacaatggaggaactaagtgtattgataaacgacatattctagagcctccccttctttcgcactctctcatacttaa

SEQ ID NO: 77 - DNA, 150 bp, Lycopersicon esculentum
tcgattatatggattgggctcgcaagaatggccctttcttcgtaaatgatacattagttttcaagtatgatgcaccaaacgcaaatggaggatttccacacagtgtatacttattaccaaactattggagctttatcaagtgcgacttaa

SEQ ID NO: 78 - DNA, 182 bp, Lycopersicon esculentum
ttaagttcaagtggaaaggttatagatataactcctggagttagcgccatgtggacctttgcggtgaagaaattgaccttctcaaagaaacagctggtgccactgcttctgattccgaagaaggaaaatctggaattgattgttgggcttgtgaaggaagggaaactgaaaacagtaatcga

SEQ ID NO: 79 - DNA, 90 bp, Lycopersicon esculentum
ttaacgcctacattttatgattgcacttgcccaaatgtcatacacattgtacgtgatgttatggagcaaattcaacgtagagatgttcga

SEQ ID NO: 80 - DNA, 92 bp, Lycopersicon esculentum
tcgattccattatggccaccctgatatttttgacagacttttccaccttacaaggggtgggattagcaaagcttctaagattatcaacttaa

SEQ ID NO: 81 - DNA, 77 bp, Lycopersicon esculentum
tcgattggcggtgcgaacaatggaaatgaagccatttgtgatgaatgcgattgaattagcaatttgctgaaacttaa

SEQ ID NO: 82 - DNA, 121 bp, Lycopersicon esculentum
tcgattggcagaacaatcaaaatgaaggatcaagggatagagcacctggtttagggacagaggccattgagccaccaaattggccattacaattctgtgaagtttgggataccggccttaa

SEQ ID NO: 83 - DNA, 90 bp, Lycopersicon esculentum
ttaagggccgtcagggagacttttacatcttggtcatggacatgctgggacccagtttgtgggatgtctggaattctttaggccaatcga

SEQ ID NO: 84 - DNA, 147 bp, Lycopersicon esculentum
ttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcga

SEQ ID NO: 85 - DNA, 164 bp, Lycopersicon esculentum
ttaatgccaatgggtgcagtatttgcaaggtacttgaaggttttcaaagcagcaaatccagcttggttttatatacatgttgcttgtcaaacttctgcatatattgttggtgttgctggatggggaactggtctcaaacttggcagtgattctaccggtatcga

SEQ ID NO: 86 - DNA, 150 bp, Lycopersicon esculentum
tcgataacagtggatatcattatcaatttgtctttgtggattccagacaacaattacttttgagaccacgccgcactgatttcctcagtatattacatgctgctcctttcttgagttccatcaccaccaagttatatagaacctgattaa

SEQ ID NO: 87 - DNA, 145 bp, Lycopersicon esculentum
ttaatcgcccaattcagctgcaccagtccattctcatagtattttcttctcagaagggaccacactcatattgtatacaattgggggactctttataatattcagcaagagaaatgaaggcgctttctcacccttcttgtatcga

SEQ ID NO: 88 - DNA, 106 bp, Lycopersicon esculentum
ttaatccctaagtaattgttagcaagaagtttgaatccacaaggtgtgcaatatgtcatgtgaatgtgcatgtccattctaccaggtcgtaacaatgcaggatcga

SEQ ID NO: 89 - DNA, 97 bp, Lycopersicon esculentum
ttaatctgttgatctttgtgagagtcttttgttttgagctatgtgatcaatgacggaactagagcggcgaactggtattcacctgagcccccatcga

SEQ ID NO: 90 - DNA, 164 bp, Lycopersicon esculentum
ttaaatcatcagcttttgagagaagacatggcaaacaaatcttacgaggaggccattgtttccctccagaaccttatcagtgagaagggagagctgggaccatttgtagcagaaagaattgatgaaatgacagctgagttacaaacaagcagtaaaccgttcga

SEQ ID NO: 91 - DNA, 323 bp, Lycopersicon esculentum
tcgatgtaactgcgtaccgatgtaactgcgtaccgatgggagatgtgctgtggaatcttgagtttgctctgcaacttcaggagagtgcagaggagtgtggaaagggttttgggaaaatggacattgaagaagggtttgatgatgttacatgcaaaggaaagaaggatttgaatgaatccccaggttacgatgcaagcatgactgattcaagaagcagtggaatatctatgagcattggcggccgcagccttgccagcgaagactcagatgggctaacacctagtgctgttttctctcaaatcatgaatccaaaaggacattaa

SEQ ID NO: 92 - DNA, 104 bp, Lycopersicon esculentum
ttaatgtttggatacaggtagcccagcgacgtgggtgcgctgcaagtcgtgcaccaaaggctgcgaatcaaacaatcgtttgtacgatttttcaatatcatcga

SEQ ID NO: 93 - DNA, 78 bp, Lycopersicon esculentum
tcgaggtgaaccagtttttggttgtgacttctcggtattttcggtgacagctaagttctataacaagagatggtttaa

SEQ ID NO: 94 - DNA, 90 bp, Lycopersicon esculentum
tcgagaacatagctaatcgtccattcttgatttccttgaccttcaactcagcaaatgcctccggatcatcagcaaggcccagtgggttaa

SEQ ID NO: 95 - DNA, 165 bp, Lycopersicon esculentum
tcgagctcgtcgcatagtgtgatttgaacggttctatttagctgtttgacattattctgagttacatatccttataaggatggaaagctaaagacaaaggactaacggccctaattagcacagccattgattttcacctatagaagtgagatacatttcttttaa

SEQ ID NO: 96 - DNA, 107 bp, Lycopersicon esculentum
ttaaaagtacgagctccaacattttctccgatcaaatcactccattgatctccaatattaccaataattctatatccagccttcaccaacgctgttctcttgctcga

SEQ ID NO: 97 - DNA, 103 bp, Lycopersicon esculentum
tcgagccgagtcactgtaccatccataagattagccatgtcaaaattgttgaagcctgatccggaattggctgaaacaatttatagcaaatgctcaagtttaa

SEQ ID NO: 98 - DNA, 72 bp, Lycopersicon esculentum
ttaaacttcgtcaattgacattcctatcgccctatatgcctatacattgtggttgcttcaccatgagctcga

SEQ ID NO: 99 - DNA, 68 bp, Lycopersicon esculentum
tcgagcacagatagattttgcaatgatgctggtatatgaccttccaagacattatgagataagtttaa

SEQ ID NO: 100 - DNA, 82 bp, Lycopersicon esculentum
ttaaagctactaacaaaagaatgctcatggatatgcgacttagctctgaaatagagtcccagcagtacaaggatgagctcga

SEQ ID NO: 101 - DNA, 90 bp, Lycopersicon esculentum
ttaagatgcacgccctccgcgtcttttctcttctagaattgcagcctcattcaacatgtcagctgcatctttgtgcatgtctagcttcga

SEQ ID NO: 102 - DNA, 79 bp, Lycopersicon esculentum
ttaaacccagtcaaaatatcttagttgacagacccatatatccacccaatctctttaccccattcaatcttgtcctcga

SEQ ID NO: 103 - DNA, 208 bp, Lycopersicon esculentum
tcgaggactttttcacctttgctgagcagcaacaacaacgcctttttatggacaagtacaactttgatgtggtaaatgatgtaccgctttctggccgttatgaatggattagggtgagtcactaagctggtggggctgatgaaattgctggatatttgatgttctctcccataactaaactattgtataaataggtggtgtagtttaa

SEQ ID NO: 104 - DNA, 368 bp, Lycopersicon esculentum
tcgagggaagaattcatgccatttataaacaccgatgcgaactctcatgatatcttttgttggacctacaagaggttgtatctctacgtaaaaactgaatgccaattttgcagccaggaggagaacccaaagtagtgtgtatttgaaaagagaaaatgcatcttcatgcatgcctcttccaacataaagccgaggctgggaccaccacattaccaaactcacaatcttgtaatctgacctctcaagaaaacggcggatgaaagggaacacaaacagcaatgcagatagcatgtttggtgacaggtaaaaaagaaccgctatgatgaatagagaaggcgaacttgaaccattcccaaaccagtttttaa

SEQ ID NO: 105 - DNA, 224 bp, Lycopersicon esculentum
ttaaaacacatattcataatcacttgctcacatctggatcacttagcatgcataaactattacaaccaaggctcatctgtcaacaaacataagacacattgctcatggagaggagccacttgctacatcttcattattcttagaaaattctattgcgtcttcatcctcatccatatgattctcatgatttgttgcagcagcaataacagagtctagcacctcga

SEQ ID NO: 106 - DNA, 316 bp, Lycopersicon esculentum
tcgaggtgaaaagaagtaccactccttttctccatacaatgccaaatctaaaacacaaaacacaaacaaaaaaatttagatccatctgaatttcagaaattttcaactgttcagtactaacaaaaactaatttaccaggaagatcccatggatcaaacttgtagagatcaatttctgctacaattggtacagcaattggctgtgaagtgcattttctgcataaataatgcgtcacaagttcttcatcagttggatgaaatctgaatcctggaggtaactgcaattcagctgctgtcattttttctttttcttttaa

SEQ ID NO: 107 - DNA, 89 bp, Lycopersicon esculentum
tcgaggaggtcgactaagctgttttcgctcctcaatcctcatgtttttggtttcgtcagttagttttatccccaaaaactccattttaa

SEQ ID NO: 108 - DNA, 82 bp, Lycopersicon esculentum
tcgagtgccaaagatgggtcatcaatgcgccgaggggcactaatatggcacgtatatggggtcgtactacttgcaatgttaa

SEQ ID NO: 109 - DNA, 112 bp, Lycopersicon esculentum
ttaaacacaaaaagaagaagcaagaccccttttgaagcttcaggtctacctagtcataaaatgcctcttggaggcaacctatgattcagacactacttttgaatggcctcga

SEQ ID NO: 110 - DNA, 413 bp, Lycopersicon esculentum
tcgagtactgataaattttgaaatgatgccggtatatgaccttccaagacattatgagacaaattcaacgtacgaagtccaacaagatctccaatagtgcttggaatacgaccttcaaatctgttctttgagagattgataatcatgttagaagtaaaaattctaacagaatcataatcttgtccctttgtagtaattgtcgtcaaataattgtaataaatatcagaaatatactctggtgttcttgtactctcatcaatttttttcatggcttgcaaattccccaaaatactttcgggtaaattcccactaaatccattagatgatagatcaagaatttgaagacgcgtaaacaagtttgtattccctgaagatttgatgggaccatgcaacttatttgatctcaagtttaa

SEQ ID NO: 111 - DNA, 127 bp, Lycopersicon esculentum
tcgagtaaaaccaaggagtgccctatctatgagcagctgtgcacaatatttgctgactcgggtgctgatgggaagtatgcccaatcaagtcactatgaaggaggagccaagttgccactgtctttaa

SEQ ID NO: 112 - DNA, 438 bp, Lycopersicon esculentum
ttaaaggttgggtggaaatttcttttaccgatttctctaggtaatctattattgacaacctcgttccaacttctttcactgtaaaagaccacaatatcctagattcacgacttgtctcaaacaagagaaagaaacaagcataaaatcttttttatagatattcaacatatgctccctatgataactgaattcataaattatggtcaacaaacaatacgagccgccagatatatcggccaaggtttcatgattaccctgtcccacgcaaatcgtttacctgtaactattcaatacccctacgaaaaattgatcacatcggaacgtttccgaggccgaatacactttgaatttgataaatgcattgcttgtgaagtatgtgtgcgtgtatgtcctatagatttacccgttgttgattggaagttggaaactgatattcga

SEQ ID NO: 113 - DNA, 220 bp, Lycopersicon esculentum
ttaagagaccccctccttgctttgtgaatactccacactttaaacagtactcctcattgagactgattatatagccgtatctaccttatgtcccccacactgacgcccaattccacatccctgtggagggccccattagcgctataagatatggccccacaatgtcgtgtgccccggtttacaactgtctcacgtgaaaaaccctccccccttaaatcga

SEQ ID NO: 114 - DNA, 91 bp, Lycopersicon esculentum
ttaacacggggcctggtaatggcggtgctataactggtgctctcttcctcaaacaatttgttgacgagaaggttcagtggttgcatctcga

SEQ ID NO: 115 - DNA, 91 bp, Lycopersicon esculentum; identical to SEQ ID NO: 114

SEQ ID NO: 116 - DNA, 415 bp, Lycopersicon esculentum
ttaaaggaaatctgatctcccttgtgtggagcatatttttagttgctaataatttatgtgctgttgatgccctttgcataatgtagtcactgccatagaccttgtttccttggcatctattgctgtcttctgagttctctgcattgcatggagtgtaccatgaagtctttcaacatcatcaatggtgcctagttcacatgctacacttcctagtttgtgtacattagggtagattttagtttgttgcttgaaaatgttattatgtgttatcttgattggcttggaaagatgagtctcttacatagcttttctcttctctttttttttcttcgtatgaatccgctccgagtccataatggatttcttctgttacattctctcgttgagccgtaaaggctcaaacctctactctcga

SEQ ID NO: 117 - DNA, 252 bp, Lycopersicon esculentum
ttaaagtcattccgaaacatgttggggaactttggaacgtacaaaccctcattgtcaacacacaacagatcaaccttgatattcaagcagacatattgaacatgccccggctgaggcatctgctcaccaacacgtctgctaaattgcctgcgcttgctaaccccaaaacaagtaagactaccttggtaaatcaaagcctgcaaaccctctccacaattgcaccagaaagctgcactgagtatgttctctcga

SEQ ID NO: 118 - DNA, 130 bp, Lycopersicon esculentum
tcgagaacatatagtctaccttttgacctaataagaaaattatcccactattcctttgatatctaccagattattagctaaatgtccttgtacatagagtacggtcagacttctcctcaaggttatttaa

SEQ ID NO: 119 - DNA, 127 bp, Lycopersicon esculentum
tcgagagtctggacctatcccacaacagcttgaatgggacaattcctgttgacctacttgagctacactttttggcaatattcagcgtagcatacaataacttatctggtgcagtaccaccatttaa

SEQ ID NO: 120 - DNA, 256 bp, Lycopersicon esculentum
tcgagctatcgcattatttccacattttggggtgcgttaggtggtgatgtatacctaggtaaatctccaagatcatctgccccttgtctagatggtgtgtttcgttacaattctgatgttggaactgtcggtacacccgttagattcattcctttatctggaggtatatttgaagatcaactaatgaacctacaattcaacattgcaacagtgaaattgtgtgttagttatacaacttggaaagccggtaatttaa

SEQ ID NO: 121 - DNA, 289 bp, Lycopersicon esculentum
tcgagcagacaaaagcggcactccccactcttggtcgccttatccactcaaatgatgaagaagtcttgacagatgcatgctgggctctttcatatctttctgatggaactaatgacaaaatccaggctgttattgaagctggagtgtgttcccgccttgttgagctgctacttcattcttcaccatctgttctcattcctgccttgcgcactgttggtaacatagtcacaggagacgatatccaaactcaggttatgattgaccatcatgctcttccctgccttgttaa

SEQ ID NO: 122 - DNA, 74 bp, Lycopersicon esculentum
ttaacaaaaagaactgtgtttttgacaatttcgtgatggagaccgaagtgcttgtaaccttatactgggctcga

SEQ ID NO: 123 - DNA, 117 bp, Lycopersicon esculentum
tcgagctgcagacgaacgggatgaagcagatgcaacaatctttacaacttcatcatttgtaagtacatcccatatcccatcagttgccagaacaatgaactcatcttttcctgttaa

SEQ ID NO: 124 - DNA, 158 bp, Lycopersicon esculentum
ttaagccaaaacaagaaaccgaagcatgatgttccaagctctagccgtgcaaatggttctgaagactcaactattactagtaaagatccggagaatatgatgacgacaaaagattaccctctagctctcttaggattcccgttgttagagtccttcga

SEQ ID NO: 125 - DNA, 310 bp, Lycopersicon esculentum
ttaaagtttggtgctctgagttccatgttcccgcaaatcagacatgtgatggttgagcaatcaaaagcgacacctagtgtcttctcaagatatggagttcacagctttccatcaatattgattgtgactcagacatcaagagtgcgatatcacggccagaaagatcttccctcccttgtgaacttctacaagaggactacagggcttcatcctatggttgaagtgaccgaaagtcaaattacctacaaggcagatggtcgcaaagcttttcagaaatggaaagggtcctctttgaaggaactatcctcga

SEQ ID NO: 126 - DNA, 192 bp, Lycopersicon esculentum
ttaaagtctcaatgataatctcatctcataccatctaaacgaatatcacaatttgggcataacccttacatcaattcaacaagaccatgtataggtcgtaaaaaagaacaatattcaaataacaagaaaaggaatgatagagtcaataagctcataacaaaatccaaagctacattatggcactgtcctcga

SEQ ID NO: 127 - DNA, 92 bp, Lycopersicon esculentum
ttaaatcattagaaaccatgtttgaaacagagaccaaacttgaaccaagaaagaagcttcaactagccagagaactcggattgcaacctcga

SEQ ID NO: 128 - DNA, 159 bp, Lycopersicon esculentum
tcgagtgcatgtgttttattcatattggatgagataagaaaaagatctgctagagatgggctgaagactactggagatgggctggacttgggagtccttttatcatttgggcctggccttacaattgagaccgttgtgcttcgtagtatgcccatttaa

SEQ ID NO: 129 - DNA, 105 bp, Lycopersicon esculentum
ttaacaaataacaactctagaatgcaacactgacaaagggcaagaagattacctggttctgcgcagcgacgagtcttccaagacgactccacctcttcaactcga

SEQ ID NO: 130 - DNA, 227 bp, Lycopersicon esculentum
tcgagccgactcataatcatgtccctttgttgtaattgtggtcaaataatcataatgacgagcgaattgatcagaaacataatgtggggttcttgcactcttatcaatttttttcatggcttgcaaattcccaaaaagacttattggtaaattaccactaaatccattggatgatagatccagaagttgaagctgcgtaaacaagtttgtattccctgaagatttaa

SEQ ID NO: 131 - DNA, 189 bp, Lycopersicon esculentum
tcgagcacactttcaaacaaccaactagtgatgtccaccaacatattgggtatttgcatcagcactgccctgaacctgatgtacagcataaggcgcagggtacagattctctccatatcctgtttcaggatttccataattggcagaatatgtaaaattcggagttgctgcagaaatctggcgatttaa

SEQ ID NO: 132 - DNA, 159 bp, Lycopersicon esculentum
tcgatccatgtgaagggtcagcgcgcatcatccctaaacaccttcagattcatgctgagcttgggatgaagttagtgacttgatgttcttctgtctaagcaaagcacttatatattgaagttgactacccgtgatatcgacttatccccttccatttaa

SEQ ID NO: 133 - DNA, 91 bp, Lycopersicon esculentum
tcgagatgcaaccactgaaccttctcgtcaacaaattgtttgaggaagagagcaccagttatagcaccgccattaccaggccccgcgttaa

SEQ ID NO: 134 - DNA, 170 bp, Lycopersicon esculentum
ttaactacataaatcaatgcgagtagcataataccgactatggttcagcgtacccaattattcccaaaactactagtcaagtatgttgtaaatccctctatgggcgatcagtttagtgatcacgccagcatgcctttatacctctggtagggtatattgggtcctctcga

SEQ ID NO: 135 - DNA, 228 bp, Lycopersicon esculentum
ttaagaaggactattacgcgattcttggtctatagaagggttgttcagttgaggagattaggaagtcgtacaggaaactgtcattgaaagttcatcccgacaaaaataaggctccaggctctgaagaggcattttaatttctgtttgcatcaactggggtagagttttttctcctgtcaaacgcacagttttggaaggtgttgactggggatgtgtaatgtccttcga

SEQ ID NO: 136 - DNA, 78 bp, Lycopersicon esculentum
tcgagatccaagaggaatacgtgaaggacgaactgaagaatctccgccgtgaacatttacgggcacaggaagagttaa

SEQ ID NO: 137 - DNA, 83 bp, Lycopersicon esculentum
ttaaccataatcatcatggattttgatgccggaatttccttgtctgtgactgaaggaccttcctctttgtcaccgtctctcga

SEQ ID NO: 138 - DNA, 423 bp, Lycopersicon esculentum
ttaacccatccaggacatctagtgaaatgctacccttggttgaactctgagcagaagcgctctcctcatcaccaaagtcttccacatgagaagcaggtgttccttccagcagataccgaagaatatcccgcttggataaacagacaccagcaccagcctcataatacaactcaacagcatcacgagtgtaagcattttctttgtcccaaggaagaggtggacaaccttctgaaaacatcatatcaagatgagctgaaaacatgtcggtctcacagaactcctctatgaagtcactggacatgacctctggatacagaagaagaactgcccaatgaagaatattatttttgtctaatgttggttttcttagtccagtgagttcttgatacatgggcttcgcaatcttcaatcctctagtctcga

SEQ ID NO: 139 - DNA, 170 bp, Lycopersicon esculentum
tcgagaacccattttcaatgtgaaaattgaatcgtatttggtttgagatctctaatgtattggaaaaactgcttaccggagccggcgacttgaaagaggtttccgacgaccggccaacctggaggtccgggaggtagatttagggttttggatttgggttttagggttaa

SEQ ID NO: 140 - DNA, 90 bp, Lycopersicon esculentum
tcgagaacatagctaatcgtgcattcttgattaccttgaccttcgattcaccaaatgcctccggatcatcagcaaggcccattgggttaa

SEQ ID NO: 141 - DNA, 133 bp, Lycopersicon esculentum
tcgagagggtgggattaccatcgtggtccatgcctatgggatcacggaagccagtggaccagccctggttgtgtgagatttcagtaccatgtggaacaattttagcgtggagaagatcactcccaggttttaa

SEQ ID NO: 142 - DNA, 230 bp, Lycopersicon esculentum
tcgaggtcacaatcttgtattggaaatacagtggacattgccttcaaatccaaaacatccaacaaagacaagaatgtggagaggagtgatggcatcttactcttttgttgcaatgtgtgtatttcccttggcaattggaggatattgggcttatgggaatatgatgcctggaaatggaattatgggtgcaattgcaaagtaccaccaagagagtacaccaaaatggttaa

SEQ ID NO: 143 - DNA, 138 bp, Lycopersicon esculentum
tcgaggaaatcaagtaacaaatgttgaaatttgcagctagactatactagctacctacctactatgttgtaaaataaacacctgctaagggatatttagcatggttttctaaataaactatctttccttctcggttaa

SEQ ID NO: 144 - DNA, 154 bp, Lycopersicon esculentum
ttaacctcctctttgtttatcccaggaacatccactttgaagacgtgagcttgtggggtctctttccaatcaattttcgcatttgcaaaagcagagatttcacgagcagaggatggatcgttggcaatggtggttgaaattgggaagccctcga

SEQ ID NO: 145 - DNA, 220 bp, Lycopersicon esculentum
tcgagtcggcctgcgcggaagatgtaacggggctcaaaccatacaccgaagctacgggtatcatcttcggatgatgcggtagaggagcgttctgtaagcctgtgaaggtgagttgagaagcttgctggaggtatcagaagtgcgaatgctgacatgagtaacgacaatgggtgtgaaaaacacccacgccgaaagaccaatgtttcctgcgcaacgttaa

SEQ ID NO: 146 - DNA, 632 bp, Lycopersicon esculentum
  misc_feature (319)..(): n can be a, c, g, or t
ttaagacagatccgaggttccatagcagtgttattgatggtttacaagaggatgcggagggctactttgcggggcgctttgaaaagaccattctttggggccgccctgcgacgagggggactattatgctccgggatattatggttgctgagaggagtgtgtaccctctaaacgggggcgcgttcttaattactggtggggggggcctatatgggggctttaatggggctgggtaaccacagtgtggagtgaggggcgggttttattacacagctgggggggggggggagaagaattcacctctcgcgggtgaaatatntntcctacgagtattttgagggtcacacacctcggaccattttatcttgcactgtatcggtggcgtgtggaaagaagattttctgtgcgttgtcangtgatgtgctgctcatgcgacttcctcacttcgttctattctgctcgcgcttgctttgtggtataatcggctacggactatgcctggagcgcgtcggtgagatcgatatgacgctgccagtttgctacactgataaatctttgtagttgtgggatctgtggttacttggcgatcactgggtttttgtcgtatactcgatattgcggtctattgtcga

SEQ ID NO: 147 - DNA, 187 bp, Lycopersicon esculentum
tcgagtaccctattgtttcaattgaagatccatttgaccaagatgactgggagacctatgctaagctcactgctgagattggggagcaagtacagattgtcggtgatgaccttcttgtcaccaaccctaagagggtcgccaaggcaattgcagagaagacttgcaatgctcttcttctcaaggttaa

SEQ ID NO: 148 - DNA, 116 bp, Lycopersicon esculentum
ttaactttcacgacaacgcctttaggtgaagctatctgcaccgtgtaagatgatttggcatcaccaacattggtcacagttctggtgtatgtctgaggagttgatccaagtctcga

SEQ ID NO: 149 - DNA, 91 bp, Lycopersicon esculentum
tcgagatgcaaccactgaaccttctcgtcaacaaattgtttgaggaagagagcaccagttatagcaccgccattaccaggtcccaagttaa

SEQ ID NO: 150 - DNA, 79 bp, Lycopersicon esculentum
tcgagaatgtcaatgctgatggtgctaaggatcttatggctcatttggtgaagctgcaatcttccattgatgaagttaa

SEQ ID NO: 151 - DNA, 412 bp, Lycopersicon esculentum
ttaactgttctttccgggccttacgaacatcagtggaattagtatctttggttactttaggttcatacgccctgaatcttctttttggagctccacatactgggcagaagtatttgtcaggtaatttctcaaaaggagttttgtcattgtaaatatacccgcaatcccggcaaatataggcttgcttggaggtaacgcgcatggagattttcggggcagcagcaggagctgctctagagaatgatggaggagggagcaagagatgaattgatggagaaaaaaatgaagattttagggctaaaccatttggtgcctgtctgaggccaccattgggggctaaattagctggaattctagtgggtacttgcagggcggccattgcttatacaaattgccctttcttctagctcga

SEQ ID NO: 152 - DNA, 154 bp, Lycopersicon esculentum
tcgagcatggaaacttattcaaaactagattgaataacatacatgcttcactaacatcgccttacaagggttatacatcatgtttatcgcaacacaatgccgcggtcccaagtttccctagaaggttcattacaacaggtgctacttcagttaa

SEQ ID NO: 153 - DNA, 431 bp, Lycopersicon esculentum
tcgaggaaaagaactctagtagtatccccctcggtgtgcatggtcattttgcaaatggagaggcaagttttggacctgcatctggtttgatttcttactcaggtcacatagcacattcgggtaacatctcccttcgttcagatagcagcacaaccagtgccagatccttcgccttcccagtattacaatctgaatggaacagtagtccagtaagaatggcgaaggcagaacggagacattataagggttggaggcaaagccttctctgttgtaaattctaagtcctttttgtttcttttcgtagagttttcagttcaaaaatatacatcatttcatcatcctttgtcagttacaagttgatattcttaggaatgaatcacatgtcattggtaataggttcaaagggctctttgagtgtgtaatcatcttaa

SEQ ID NO: 154 - DNA, 211 bp, Lycopersicon esculentum
ttaagattgctatgggtgctgcacgtggactggatttccttcatacatctcaaaacagggtgatacatcgtgatatgaaaacctcaaacattttgttggatgaaaactgggaaagtaagatttcagattttgggttgtccaaaatggggcctggaaatgaatcagctactcatgttagtacacaagtcaaaggcacatttggttacctcga

SEQ ID NO: 155 - DNA, 130 bp, Lycopersicon esculentum
tcgaggatgaagataaagccatagtgctcctaaactcgttgccatcttcctacgatactttatcaacaaccattttacatggtaaggattctattgagttgaaggatgtcacatcagcccttttgcttaa

SEQ ID NO: 156 - DNA, 246 bp, Lycopersicon esculentum
ttaagccattcatcctccaaaatttcaggcacggtaatacgtgtcgtaggattcggatccaagatgcgagtaatcaacttgatagcaccaaaggagatccaaggtgggcacgtaaattcagcagcagagattttgttatagaggttcataagattggagtcatcaaaaggcaagtaacctgcaagcagtacaaagagtatgactccgcacgaccagaggtctgctgttgccccatcgtatcctcga

SEQ ID NO: 157 - DNA, 35 bp, Lycopersicon esculentum
ttaagtagacagatatcactgattgcttctttcga

SEQ ID NO: 158 - DNA, 89 bp, Lycopersicon esculentum
ttaagaagaaggatagtatcatgtgaatgtgtatagattactgccaattgaatagaatcactattcagaacaagcatcccttgcctcga

SEQ ID NO: 159 - DNA, 122 bp, Lycopersicon esculentum
tcgagggatagggaatttattgtcttctaccgagggaaggacttcttgccctctgcagtctcttcagcaatagaagaacggagaaagcaagtttttgaagaggaaaagcggaatggtcttaa

SEQ ID NO: 160 - DNA, 305 bp, Lycopersicon esculentum
ttaactgcaatcaactcatctaacaaagttcataccaccttctacaatcatttcagcagtggcacccatagcgccaataccactactactcaaaagagccaacgctaactcctcctcgccccactgtttcaaacccatactgatactcttcaaaacctctacatccacctctagtaaccagtttggtgtgcatctaactatcaagctgatgagcccggagacataagatttccaagttgatttgttgcaaccgagggatattttcccatgtagaacacctgcaacgaactccaagtgtgcctcga

SEQ ID NO: 161 - DNA, 117 bp, Lycopersicon esculentum
tcgagtcaacgcgtttcggagaatgatgaagaaccaacctattgctcggattgataaagaaaaacaataagataaatcttgaaatgtgcttaggatgctgagaattgtacatcttaa

SEQ ID NO: 162 - DNA, 87 bp, Lycopersicon esculentum
ttaagatttcgctcaagaacatcctagactatgcaatatattctcagaaccaatgatgcaagcaagaactgaatcccatttactcga

SEQ ID NO: 163 - DNA, 242 bp, Lycopersicon esculentum
tcgagtaggacggagcacgagaaactttgtctgaatatggggggaccatcctccaaggctaaatactactgactgaccgatagtgaactagtaccgtgagggaaaggcgaaaagaaccccggagaggggagtgaaatagatcctgaaaccgtatgcgtacaagcagtgggagcagactttgttctgtgactgcgtaccttttgtataatgggtcagcgacttattttcagtggcgagcttaa

SEQ ID NO: 164 - DNA, 151 bp, Lycopersicon esculentum
tcgagttgaagaggtggagtcgtcttgggagactcgtcgctgcgcagaaccaggtaatcttcttgccctttgtcagtgttgcattctagagttgttatttgtttagattcaatatgattagtggtgtgaatgacagtggaagtcttcttaa

SEQ ID NO: 165 - DNA, 279 bp, Lycopersicon esculentum
ttaagacgagctctgaaacagtatacccctcgtatagaagtggatttgatgaattttttgcaaaataaaaaagaaagacaacgttccaagtacgatccatatgacattactggcacatcttctgaagagggatatgtaggagcttcaaagaaaaataatttatttggaaggtattctgctggttcagttgatagtgatggtgccagaaagtggaattctgttccagacccgacctatatggctagttctgtaggtcactcattgtcggatgatactcga

SEQ ID NO: 166 - DNA, 137 bp, Lycopersicon esculentum
ttaactgcctgcaacggtatccttccccattccaattatctttgcttttattccagtgaaaaacttgaacaagaatcaccaacgaatctgctttctctgaagtattggtaaaattccttccagacatctccactcga

SEQ ID NO: 167 - DNA, 185 bp, Lycopersicon esculentum
tcgagtagttgtgatgaaaaactgagatccatttgtgttaggtccagaattggccatagataacatccccttcctttcatgcttcaattcaaaattttcatcctcaaattttagcccatagatggattctcccccagtaccatctccagcagaaatatcaccaccttgcaccatgaagctcttaa

SEQ ID NO: 168 - DNA, 251 bp, Lycopersicon esculentum
ttaaggaaagtaaagacctcaaaacacaaaatgctactccacttcaaaacaactttacataatatgcttgtactattatgtagtctatcttgctgtgacttgccttggagatagtccctcctctgaaacatcagaaaatagatcaatacttttagacaacgcatacacaatttcaatcatgcctggccttcttgaaggatctttattcaaacatgaaatagccacagacatcacattcacaacactttcga

SEQ ID NO: 169 - DNA, 301 bp, Lycopersicon esculentum
ttaagtcgtctcgtcgtaaaatccgtgttgttgacattggttctttggagaaaatcagatctgggaaagttgaggttgttcctggaatattcaacaagttttcatgtgctactagtactggcttacgctctgacccgagtagcatggggcacgtggaatcccgtgtgaatcagcaaggaccacgcaatgttccgtattggccaaagtttaggttcagattgttgaagatttttgggaggcgtcattgttgaaggcttggattcttgctttcatccaaggagggtaggtcctcaactctcga

SEQ ID NO: 170 - DNA, 226 bp, Lycopersicon esculentum
ttaatactagcaacatgtcataagtatacatcattttagtaaggacgttgtccgatccagttgcctacaggatcatagttgcaagaaatgaaccaccatccgttgttgcaacgtgcccgaccacaacctagtcggactgagttgcgccagactacttgagtataatgtctacacttttttccaccaacacattggttggtagcgtagttatagcttggcctctcga

SEQ ID NO: 171 - DNA, 91 bp, Lycopersicon esculentum
ttaatacggggcctggtaatggcggtgctataactggtgctctcttcctcaaacaatttgttgacgagaaggttcagtggttgcatctcga

SEQ ID NO: 172 - DNA, 103 bp, Lycopersicon esculentum
  misc_feature (19)..(): n can be a, c, g, or t
tcgagaaccaccatccttnctggagacttgttgagcttagccttgcagagtggctttatgctctactagggagcacgggcgctgcaatatttggttcccttaa

SEQ ID NO: 173 - DNA, 123 bp, Lycopersicon esculentum
tcgagaatgaggttgagttcactaacataaatggcctcaatttggtgttgatcttccattcatgttatcccgaggattctcaggaggttgagctcctgaaaacatctccataacagaccttaa

SEQ ID NO: 174 - DNA, 131 bp, Lycopersicon esculentum
tcgagatggacggccaaaaataaagttactctcccagtgtgttggtcgtgccttgagcgtaactctgtgctaggtggtgatttgttgaatctaatattggataacctatagctttgtcactctgtacttaa

SEQ ID NO: 175 - DNA, 351 bp, Lycopersicon esculentum
ttaatactagcaacatgtcataagtatacatcattttagtaaggacgttgtccgatccagttgcctacaggatcatagttgcaagaaatgaaccaccatccgttgttgcaacgtgcccgaccacaacctagtcggactgagttgcgccagactacttgagtataatgtctacacttttttccaccaacacattggttggtagcgtagttatagcttggcctctcggacacccacaattgcacggctgccctccccgtgaagtcaccaccacccttggcaagattctccccagcaccagaatgaatcaagttacaatcaccagctcttgagttggcatagttttgtgctcga

SEQ ID NO: 176 - DNA, 105 bp, Lycopersicon esculentum
tcgagcatatgcatagctttgatcctccttatgcacataatgatgtcaaacctggtaatgtcctgctaactcatagaagagaacaaccacctcttgcagtattaa

SEQ ID NO: 177 - DNA, 107 bp, Lycopersicon esculentum
ttaaggcacggtgaatgactatcaatgtttttagagcaaccgcccaattatgtgtctttgacaaccttctcgcaagagcatgaatgcagtatgcaacatcagctcga

SEQ ID NO: 178 - DNA, 155 bp, Lycopersicon esculentum
ttaagtcatttgcaaccttgagccacaaggacgacttcctatccatttttgcaagcattccagcaatcagaacaaccacaagcggtaatcccttacagtgtagggcaacttgcaaccctgcttccagtagatcggggggacaactctctcctcga

SEQ ID NO: 179 - DNA, 265 bp, Lycopersicon esculentum
  misc_feature (30)..(): n can be a, c, g, or t
ttaaattgaacaattgatgcattgatttanaaccgcgaccaatatgagatgaattggcatatggcatagcagggtttgtatattaacaaaaaggaaagattttttagtgtgaggatcgggtaatacgaatgcaaagctctggctttcaggtgcagcaaaagggtggtctcacaagtcacaacatccggcccctcaacatgggttgtgctttccttacctgcttcaggggggcaacgatgaaaaccttgtgtggcggtcccttcga

SEQ ID NO: 180 - DNA, 347 bp, Lycopersicon esculentum
tcgagggctggagatctctcttatccttgctggaaacaatccaaacaccggtttataatctctctgacaagttctaccaaggccaggatctgaaagagaggattcactttccgaaattccaagtgacatgattcctccttcagaagcttcatcttcctctgatcttgtacagagtaagagtagttcatattgttctatcacatttaggagcctttagtagagtactttatgttattagtaattattgtaaaatctggtaatgtacagttttcagatatgtaaaatcataattttgactctacatgatccctcattattattgcactgaacattatggtttgtattaa

SEQ ID NO: 181 - DNA, 125 bp, Lycopersicon esculentum
ttaaggcaagatttgctgattgtcattttgatgaatcagtatacccaacattagggggagaacataagtcattgggaaaagagatagattggaattcatcatctctatctcatttggatcctcga

SEQ ID NO: 182 - DNA, 179 bp, Lycopersicon esculentum
ttaagggcgacagactatactgatatagtgcagggaacgacatttgtgctgcaaagggaaactgaaccaaggcaaaagttgctttcgtccatttcaacgggtacaagggtagaaaaggcgattgacgagtttatttccttgaaccaaaatgttgtgaatcctgaactggaactcctcga

SEQ ID NO: 183 - DNA, 236 bp, Lycopersicon esculentum
ttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcga

SEQ ID NO: 184 - DNA, 405 bp, Lycopersicon esculentum
tcgagtttacgacagatgaggagcaagttattgatgggttggatagagctgctatgtcaatgaagaagggtgaagtggcactgctaataattgcacctgattatgccttcggtctatctgaatcaaagcaagacttaggtgttgttcctcccaactcaactgtatattatgaggttgagctggtttcctttgtcaaggaaaaggaatcatgggatatgaatacccaggagaaaattgaggccactggtaaaaagaaagaggaaggaaatgctatcttcaaggctggtaagtatgcaagagcatcaaagagatatgagaaggctgctaaatttattgaatatgatactgactttagtgaagaggaaaagaaacaagctaaagctttgaagatcagctgcaacttaa

SEQ ID NO: 185 - DNA, 215 bp, Lycopersicon esculentum
ttaagttgatcgtgttactgatggaggacggttactagtaagtaactggtaatgttgatatactcttctttagtgttggaggagccactgctgatatctccaagtgtctctcctttgccatccatccacccgattgatttccaggttgagtcggatgctcaaacactataccaatctctcccttgtgaggtttgcacacattcatcttcactcga

SEQ ID NO: 186 - DNA, 72 bp, Lycopersicon esculentum
ttaataatagtggtttcatgtctgaggaggagctaaggtctgatcctgcttacttatcttactattactcga

SEQ ID NO: 187 - DNA, 143 bp, Lycopersicon esculentum
tcgagtagctctagaagcatgtgtaaaagctcgtaatgaaggacgtgatcttgctcgggaaggtaatgagattattcgcgaggcttgcaaatggagcccggaactagctgctgcttgtgaggtatggaaagagatcgtattaa

SEQ ID NO: 188 - DNA, 133 bp, Lycopersicon esculentum
ttaaggatattagatttggatgcctcaacttcagcttctagtatcagattatgcaccagaaggaagtttacaagccatactgcacgaaaggccttcgtcttcaacttcccttcccctgtcttggtccactcga

SEQ ID NO: 189 - DNA, 171 bp, Lycopersicon esculentum
ttaaggccctcaagcctaaagcttgagtgcaagttcccactcacaactttgcgtaccaatattcttgtgcctattattcttattgcggaccaaataaagaagcttcattgcttggagctcttagaatccatgaaagtatgtagacacttgctttggtctccctccactcga

SEQ ID NO: 190 - DNA, 221 bp, Lycopersicon esculentum
ttaaggatacaacttcaaacatgaacagaatctggtttgttggaaaaataagtttactaacacaccacaacctcaaatcaaacaaataaagaatcacaatcttgaatttttacattgacatggattgtacttgaggagcttgattcacttgttggttcccatagtgtcgctgagcaactataccattccaccctagagcgggatcaagtcccctgtttcga

SEQ ID NO: 191 - DNA, 297 bp, Lycopersicon esculentum
ttaagtactggttagatgtgttcaacaccctttccatcgtattattgctgcatggtgtgcgtgatgaactcaccaactcacttctgtagccatctccaaaacgtaacgtagtgttgttttgggaatgactatgataccgactaacaactccggtaccctagtcttcccaccgtctctgctcacctactgtctcagaagtggtaacgaatgtcgtgtgtgatacactcacgccaacctgtcgctgatgatcgctgagcaagggtgtcttcaacatcatcatctccactcacttctcga

SEQ ID NO: 192 - DNA, 178 bp, Lycopersicon esculentum
ttaatgatacggacccatgcctgtgggtaagccttctttgcctcctgcacctcagccaagacctgggttgcatcagtgcacccgaacatgggcaacttccacatggtccagtatctgccatcgtagtatcctggtgacttatggttctcacggtacacaaatccgtgctcagtctcga

SEQ ID NO: 193 - DNA, 102 bp, Lycopersicon esculentum
ttaatctgcttgcagctgaccattgtccaccatccatccctacgacaaagaagatatggccatcaatgtgccatgactggacagtatcttctggattctcga

SEQ ID NO: 194 - DNA, 228 bp, Lycopersicon esculentum
ttaatcagcaataatgaccactgccaaccctatggataacactcagaagcgaatcaccgtagctactaccaacgaaccaacaacaccatacggcattggatcagggctcattgatcccaacagagcacttgatcccgggctaatatacgatgcaactcctcaagactatgtcaatcttctatgctccaagaatcaccacaactgctcaaatccatcagatgatctcga

SEQ ID NO: 195 - DNA, 142 bp, Lycopersicon esculentum
ttaatcgggaaaacaaacatcaacaacgtactcagtatactcctataagtggagtctagagaaggtagggtgtacaatgggcttaccactacctttgtattgtagaagggttgttgccgatagacccccgactccgtctcga

SEQ ID NO: 196 - DNA, 238 bp, Lycopersicon esculentum
tcgagattggtttgggataccagaaaatggaagaatacactctcaggacatcttgaatggtgtaaaattgaatgtacagcggctattgaaccacgcagatactcaacatcaagagtaatcgcaacatgaacaagcgaaggatcgcaaaccccagttttccccaattttcttctttcattagaagtgcattcaccggcatttcggaatacaggggctttacgaaatgagaaacgattaa

SEQ ID NO: 197 - DNA, 65 bp, Lycopersicon esculentum
tcgaaacttttcagtgataaaaagcttgagagaaagtgaaaatctacaggtaaaagatgacactg

SEQ ID NO: 198 - DNA, 88 bp, Lycopersicon esculentum
ttaaaatgaaattgatcggcaagctacaagatggcacagtcttcataaaaaagggccacaatggagaaaatgaagacgagcttttcga

SEQ ID NO: 199 - DNA, 107 bp, Lycopersicon esculentum
ttaaaccttgggtggaaataaattgcgaggcacaattccagcaaatattggtaatttacgaaagttgcagcaactacaattggagaacaattacatggtgggttcga

SEQ ID NO: 200 - DNA, 105 bp, Lycopersicon esculentum
ttaaagaagaaaaaagaagggggaatagagttgaagaaaggtaaattgattatgaacatggaaataagtagagtgatagatggatttgttgtcgttgaggttcga

SEQ ID NO: 201 - DNA, 147 bp, Lycopersicon esculentum
ttaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcga

SEQ ID NO: 202 - DNA, 268 bp, Lycopersicon esculentum
tcgaacccttacggccaacaatacaagcatatcctttttgaaagttggaactatcacttgagtttgtacagggtttcctctcagtttctaatgaagatggggcaactttttccacttgggtggtaattttatcttttacctcactctgcaaattagatttactgccagcaaattcgtcctgtgacgtatctataaaatcactctgatgaattttggggaccaaaattgcatatctcattagtgaaaccccagctgtcatcagctttaa

SEQ ID NO: 203 - DNA, 153 bp, Lycopersicon esculentum
ttaaagcaaggtggcactatgtgttacatatggacgggatgtcaaagaagccaattactgtatctactacacattttatattcaaggatatttcattgaaaggatttctgttacgagaaaagaatttagatgaagcacaatataagtgttcga

SEQ ID NO: 204 - DNA, 262 bp, Lycopersicon esculentum
tcgaacgatctctcgtaaaatcattttcgtttcttattcaggatctcatactcgtctacatctttaattacatcgccgacacttactttcacctcattccaaccccatatagttatgtagcatggaccacttactggattgctcaaggttgtgtaggggaaggaatatggatcctagctcatgaatgtggtcatcatggctttagtgattaccaatgggtagatgacactgttggtcttatccttcactcttcacttattaa

SEQ ID NO: 205 - DNA, 151 bp, Lycopersicon esculentum
tcgaacacttatattgtgcttcatctaaattcttttctcgtaacagaaatcctttcaatgaaatatccttgaatataaaatgtgtagtagatacagtaattggcttctttgacatccctccatatgtaaccatagtgccaccttgctttaa

SEQ ID NO: 206 - DNA, 155 bp, Lycopersicon esculentum
ttaacgcaatggtggcactatgtgttacatatgtgagggatgtcaaagaagccaattactgtatctactacacattttatattcaaggatatttcattgacaggatttctgttacgagcaaaagaatttacatgaagcacaatatacgtgttcga

SEQ ID NO: 207 - DNA, 55 bp, Lycopersicon esculentum
ttaacccgtaaacagaacttgaactcgcccaaacaatggcgggttgtgggttcga

SEQ ID NO: 208 - DNA, 272 bp, Lycopersicon esculentum
tcgaataatacatcaagaggctgatccttagtaagagccaaacgccttcttccatcttgagaaaactacacccacatttacacataactgacccttacggcaaaatggacagaaaataggagtaggatcgcaaaggactaagttgtaaccaagttttgatggaacaatcttgaaatagctgctatctctttgccctatgcttcctccagtctccaataataccccgcctagagtttcattgtaatctcccactttccaaattgtatagttaa

SEQ ID NO: 209 - DNA, 130 bp, Lycopersicon esculentum
tcgaaggataggatgaagatcatgtaaatagttcactagaatctgatgtatttctgaagatcaatgggcattctatctggggaagaacataacgtttgagtgactataagttacattcagtacatcttaa

SEQ ID NO: 210 - DNA, 199 bp, Lycopersicon esculentum
ttaaggaggagatattaccaagccaggacggtatgttacctgacaaattgttgtaactcaagtccaatgtacgtaaaggagcagtggataacgtctgtggaattggtccggagaagttgttcccatccaagtataagtaattgaaattgttgatgttggctgaagcaggaaatatttcccctttcaacatgttattcga

SEQ ID NO: 211 - DNA, 101 bp, Lycopersicon esculentum
ttaaggcgatgacacagtgcatggaatcgctctcgtctttactcatttgaaccaataaaatctccaagaaacttgcaagaggaataatcatatggattcga

SEQ ID NO: 212 - DNA, 82 bp, Lycopersicon esculentum
ttaattttaggcaaggaaagctttgagagcactaaagggactagtgaggcttcaagctattgttcgtggtagggctgttcga

SEQ ID NO: 213 - DNA, 352 bp, Lycopersicon esculentum
ttaatagaatgggagaattgttagagcttccactttctacacaaaaccatctattgcacaaaatcacattcaatgggtaaccctgaaaagatcagatgatgcctgcgaactctcacccattgtaagtccaaaaatgggatcctttgctttctccccagtcccagaaactggataagctttcacaaattttcctttgttgagccattcatcggccggacttggctcgtctgaatacttccctccactaaaagaaaagccgtccccaactgaaacaggagcatccagcaaataatcctccttatcatcactactggatcggcaattgaatcgcaaaatccttgcctgagttcga

SEQ ID NO: 214 - DNA, 219 bp, Lycopersicon esculentum
ttaacaaacttagcattgacatcaactgacgttagaaccccttcttcgtcaatcatgtagaatccagtgatatcccctacttcaccagatgaatcaaatacggagggttgatcaaacctgaatatagccataccatttgttccatcccttgactttgttagtttcacatctggtattgtttgctcatcagttccttgtatgaactgaatttttggtcga

SEQ ID NO: 215 - DNA, 275 bp, Lycopersicon esculentum
ttaaattatagagtaaaccaactaacaatacaaacatatcaaggattatattgcttcccttgcatggtggttcctctgatgaacaggtggacgtgcatccattgatgataacttatgagagatgaagtgcattgttggccttgagtttggagtttggagcgaacaagagcatgctagattgatgataaaaaccaaaacatcttttactcctatcttcaggatatggaaggcgttcagtccagcaaaatgctaagctgctcaggatctagactcga

SEQ ID NO: 216 - DNA, 236 bp, Lycopersicon esculentum
ttaagtgcagtgtacatttttcaaccatatgataaaaacacgacaaaagttctcaagatcccccaaaacacacacacaaaaaccaggaaaggaagtatcaagaccccccaaaacacacaaacaaaagcacttcacacatgataataaatcaaggaatcaagttgcaggaaactactctgatgatgcacagttctcaaatttctcatttgggattcatgacagcccaatggtgtcga

SEQ ID NO: 217 - DNA, 86 bp, Lycopersicon esculentum
tcgacccatgtgaccatactgtacctctactatcgctcttccatttcaatttgtttttcttatttatttcttctacttcaacttaa

SEQ ID NO: 218 - DNA, 351 bp, Lycopersicon esculentum
ttaatggttgagagggttgcacactattctgatatattgaagatattgcgtgacctttatcctactatgcaacttccagaaaagtgtgctgatgacaacccattgatgcaaaattatcaagtatcaaaggagaaggcaaaaagcttgggtattgagtttactacccttgaagaaagcatcaaagaaactgttgaaagtttgaaggaaaagaagttttttggaggttcatcttctatgtaaaaggcttctcaaagcttttatggttttgttgaacaatactacccaccccaccctaccctacacactttttttttttacttctttaagctaattatagaatcaagaagtcga

SEQ ID NO: 219 - DNA, 258 bp, Lycopersicon esculentum
ttaatatgatttgttggattatatgtgttgttggtattgttgtggataatttgggttgttgttggattgggcatgcacagtcacagcacacacatcaggggccagtgctgcctggcattttcgttcagcattcattcacctagcttcacatacgtcagtccagcgcgcaccatttatctcaattgctggcacgcattgttgcattgttatagactttatacctataagacagtaacatattggacagtacgagctcga

SEQ ID NO: 220 - DNA, 229 bp, Lycopersicon esculentum
tcgagcaattgtgcttagtagtagcttgtgaagaatgtaaaacttcacggtccagttccagataagtctacttttagcaaatcctagtagaagacagtagcattatgagaatctgatgattctcttttacatcaagtactttagtatgtgtactatatgttctttagggtctttacatatgcactaatggtagttcatgaagcttgaaactgttcttgtactatattaa

SEQ ID NO: 221 - DNA, 227 bp, Lycopersicon esculentum
tcgagccgtacgtccaatatttactgctcaaggtataaatctataacaagcaacaatcgtgccgcaattagataaatgtcgcgcttactacttatgtaagctagctaataatctaacgaaaatccggcagcacttcccctattttctttacttcatcccaatccaacaacaacccaaattatccacaacaataccaacaacacttataatccaacaaatcatattaa

SEQ ID NO: 222 - DNA, 134 bp, Lycopersicon esculentum
ttaatgaaagcagttatgaagtgaaatccttttctttgtggtcctccacttcccatcagttgcacggagcctaaagagataccgaaatcacctcttgaaccagaatgttgtgaagacgatactggtttcctcga

SEQ ID NO: 223 - DNA, 129 bp, Lycopersicon esculentum
ttaacagcatagtgtagtgaattccaaccccatacatcatattcttcgcataaatctcccttccaatgcaatagtgccatcgcgcattccgcgtgttcttgaatcacagctgcatgtagaggtgttcga

SEQ ID NO: 224 - DNA, 95 bp, Lycopersicon esculentum
ttaattcacagtttgcaagaactcaaatatctcccaacctagggggaggacctgctcctacacttgtcaaagctgagctaccgtggtcagttcga

SEQ ID NO: 225 - DNA, 331 bp, Lycopersicon esculentum
tcgagtgcatgagagaacatcacaaaataaaactgcagcgagtatgaaaagagatgcacaacaaaagagccctatggtaaaaacaaattttaccgttgtgtggcagaagacaattgccaggagaagcttctcccatcccccaacacagaagttatgcattttatgttcacagttcaaaggattatgtattatgacgaacaagcttcagttatcttcactaacagatctcctttgaccagaccccatttacatgtcatccgaactaggaaatttcagtcttattaggtatacatgtgtcttaacatagtccctaaaaaaggttggccattaa

SEQ ID NO: 226 - DNA, 142 bp, Lycopersicon esculentum
ttaatgtagccaccggttttcctaccagctaagacatccataagtgtggtcttaccagctccactaacacccattagagctgtcaaaactcctggtctaaaagcaccacttacccctttcaagagttcaagtcggtcctcga

SEQ ID NO: 227 - DNA, 105 bp, Lycopersicon esculentum
tcgaggcctagtctgaggatcctgccttatgcacaaagatgcagcatgcaccatgcaatacacctcgtgctccttgtagctgcttcctagacgagcatctattaa

SEQ ID NO: 228 - DNA, 79 bp, Lycopersicon esculentum
tcgaggcaaaggtgggaaatcctcacagaactcaaaggtccataagcaattgagtggttggtgaagtatacactattaa

SEQ ID NO: 229 - DNA, 266 bp, Lycopersicon esculentum
ttaatatcagctcgttgctaccaaaaccatactgccaagaatctacctcaggcccagcaatcggatcgcagaaattgaaaatcgtaccaagacatggaccctgctatccaaatgctaaaactccatcgtccgattccgagcaacttatgaattgggataaagttagagttcgttcaatcaataaaaaacctaaaactactccacttgttcttagtaattatgattacggaagctatactgtaaaaataggtcttggcacacctcga

SEQ ID NO: 230 - DNA, 84 bp, Lycopersicon esculentum
ttaatatcctcgctgcttggcctacttctgtggatgacaacaaggacaccaaatccacattcaatattatatcaggcacctcga

SEQ ID NO: 231 - DNA, 182 bp, Lycopersicon esculentum
tcgagttgtgaacaccatggtacaatttttcctattttctccaactcttcaatacaaccaattacatttttctcattttctttgtttttttcatcttgttttataatccataaaaatggtctccctatacctatcaacccttttgatatctcctccatttggcttattgatggattcattaa

SEQ ID NO: 232 - DNA, 73 bp, Lycopersicon esculentum
tcgagtagtgaagccaatgtcgtctgatacagttccgtctttcatctggtcagcttcaagttctactcattaa

SEQ ID NO: 233 - DNA, 90 bp, Lycopersicon esculentum
ttaatggagttcttgaaagtggctagttatgtaaacttgatggctattgatgcaaacacaacaatggttcattcatgtccacttactcga

SEQ ID NO: 234 - DNA, 91 bp, Lycopersicon esculentum
tcgagtatgccatcaaaggcaagtgaggtcttctcttctaacttcaatgctccaacattagcttcaagatgatcagttcccgtgccattaa

SEQ ID NO: 235 - DNA, 102 bp, Lycopersicon esculentum
ttaagggcttccccttcaatacattcttgatatggtactggatgactgtattccttgccctgagacagagcattatcttctgtgacttccccgttcattcga

SEQ ID NO: 236 - DNA, 157 bp, Lycopersicon esculentum
tcgagtatagctgcatccaatgtaagttttcgctttgcaatctcatatacattctcatcaacagtattcctagtaaccagcctgtatactgtaactggtttgttttggcctatccggtggcagcgatcttcagcttgccgatctatctgtggattaa

SEQ ID NO: 237 - DNA, 144 bp, Lycopersicon esculentum
ttaacgagtgcctggaatattagaaggaacagtaacaggttcatacttgtgcagcaatttccagacgctcccatgtcaaccttcgtctctccgctgctacatttgactttgcattttcttcttcaagcttgtgttggcactcga

SEQ ID NO: 238 - DNA, 104 bp, Lycopersicon esculentum
tcgaggaggtatctagcacaccagatatgatctccaccttcaattccaacactatgacctacttgaaatgctcactacaatgcttgcctgtatgctggaattaa

SEQ ID NO: 239 - DNA, 104 bp, Lycopersicon esculentum
tcgaggaggagctgaagaccaactctactgatccaactgcacctttggtttctctcaccaaaaacctatgattcagacactacttttgaatggccttgaattaa

SEQ ID NO: 240 - DNA, 258 bp, Lycopersicon esculentum
tcgagacgggtgtccttgtgacacgaagcaatcggaagtggttccaagaaaagcctctaagcttcagtcatacgagaccgtaccgcaaaccgacacaggtgggcgagatgagtattctaaggcgcttgagagaactcgggagaaggaactcggcaaattggtaccgtaacttcgggataaggtacgccccggtagcttgactgctttactgcagaagagtgaaagggttgcaataaactggtagctgcgactgtttaa

SEQ ID NO: 241 - DNA, 252 bp, Lycopersicon esculentum
tcgagactttagctagtactggaatacatgtagcacctataaatacatttccgtgaaccattattcccatacatatgttctaaggctgttctcttggttgggttgaatcctccgtctggtcatgcacatcttagaagatagttgaagcaataagctcttgaagaaacacagctctttgccttttctcttcatactgttcttccaaggctggaccacatactggcattttgctgctttgtatcatatgtttaa

SEQ ID NO: 242 - DNA, 229 bp, Lycopersicon esculentum
tcgagagctgttggcatagtgagccacagttacgcccaacattcctagagttggtagataaactcaaggatatgctgaggcagtatgccattcatgctcaggcagcacgtaatgttgcaggaggtaacagccagaaggaactataaataaagatcgtgttatttgcgtgaagcttttgtggaatagccagaagatacactgataacccagagccttcagtgctgtttaa

SEQ ID NO: 243 - DNA, 157 bp, Lycopersicon esculentum
tcgagggcaaatgatccatgactagtttatgataccccatttgaggattatgtaccttatttatgtggtttgaactacacaaatccacaggtaggtaaattgttacgacgcatggtgaattgctcggaggttgaaagtatccctgaagcacaattaa

SEQ ID NO: 244 - DNA, 158 bp, Lycopersicon esculentum
tcgagggcaaatgatccaggactagtttatgataccccatttgaggattatgtaccttatttatgtggtttgaactacacaaatccacaggtagtgtaaattgttacgacgcatgctgaattgctcggaggttgaaagtatccctgaagcacaattaa

SEQ ID NO: 245 - DNA, 57 bp, Lycopersicon esculentum
ttaaacagaaaagataactcttcccgagtgctacccgccgacgtctccggacttcct

SEQ ID NO: 246 - DNA, 320 bp, Lycopersicon esculentum
ttaaggggcctctttctatcttaagacaaccaatctaagcacatcaataagaaatgatatgggagatgcatgtgatgaacacatgactatcttacaggatatatcatgcgagtagggcactcttttctctaaaaaaatattatcatgaatgcgcaagaataatgggttaagggacaacaaaaagattcttcagaggaatcgggtagggggttgtcttctgggatccgcttacaaggcgggtaggtggagccttcctggccggggggtacaaatgagggcccgctaacggggggaccacaaatcctaaaacattcagtatt

SEQ ID NO: 247 - DNA, 214 bp, Lycopersicon esculentum
ttaaataaatgttgattgacataagaacaattcatattgcaaaacaaagaaaaaattcacttattatgaataattcctataataggtataaaatttccctgtaaagtatacagaacttatgcctcattacatcacagattagtcaataatgcaatggtatcaaccaaaactaaaacaacaccgcgattttttcaatctgatatcagtatatcga

SEQ ID NO: 248 - DNA, 238 bp, Lycopersicon esculentum
tcgatcacaagcaatgttctttgataagaagcaaacatccaatattatagagctgcagctttccagctctacatttacaatacaattattccgggctgctgggagcattgacttgcactatctaaggttcttacgcacttcccaaaccagtaaaggggctgctaggtgtagcttcttatatattctcctgttctgagttgacctccacccttgcaccctccatgctattatactttaa

SEQ ID NO: 249 - DNA, 372 bp, Lycopersicon esculentum
tcgattggatggacctgcaacatgtgttggttctttgcaaactttcaagatttcaatcaaagcatcatgaaaacctgactccgctgccagataaagtggtgtctcctctgccttatttgctgggaagtcgtgttcaggatctagtaataaattcatcaagatcctcacaatgtcaatgtgtccacttctaacggccttgtgcaaggccgtatctccaatgtcatccgtcatcctcacaagtactgtctcactgtccttgtatgctaagcagttcatttaccacttcactttgtcctacattagctgcaatgtgaagcgcagaaacattaaacattatcttataaagtaacaaagcccgggggtacgccttaa

SEQ ID NO: 250 - DNA, 352 bp, Lycopersicon esculentum
tcgataccaatcaacattgagtttggccaagaagctcaggctgctgctactcagaagctcaagattggactctattttgcaacatggtgggctttgaatgttgttttcaatatttacaacaagaaggttttgaatgcatttccatttccatagcttacttctactctttctctggctgctggctctcttatgatgtaggtttctatgggctactaagattgctgaaactccaaagactgactttgattcttggaaagccttgtttcctgttgctgtggctcacacaattggccatgtggctgcaacactgagcatgtcacaagttgctgtcatcattcactcatattattaa

SEQ ID NO: 251 - DNA, 179 bp, Lycopersicon esculentum
tcgatataaggtaacttagagaagtcctcttcttctacccatcgttctcttccaatggctctgtcaagttcttgttgtgccttctccgtgatgtttggactccgcaaaagttcttgaaatgcccattctattgttgctgctgaagtatcggttgcacctgatatcaaatcatgtattaa

SEQ ID NO: 252 - DNA, 373 bp, Lycopersicon esculentum
tcgatactcagaccaagcttcagcatcctgtgaattctgttgccaaatgtgtttggctcatcaaggctaaaacctgaagtgagaagagcagtctcaaacaacaatagaaccaaatccttgacggacttgtcattcttatcagcatcagctctcttcctgagctcatccatgatggcattctcaggattgatctccatggtcttcttgctagacatgtatccagccatgctaaagtccctgagagcttgtgccttcatgattctctccatgtaggcagtctaaccatactctccagtaaccaagcagcaaggagaatccactacacggtcagagaccaccactttctcaacctagacacctaaaacatccttaa

SEQ ID NO: 253 - DNA, 203 bp, Lycopersicon esculentum
tcgatgtagctggccccgtatggagcgatgaaaagaaaaacgccacgggttatggtgtttcaactctggtggaatgggtgctgaggaactagtcaagatgttgatggctaaacatattagggactaatgatgtttggaaaaataaatgcatcaagttgtatgaataaaggcaatagtataggctttcttgttatcttgattaa

SEQ ID NO: 254 - DNA, 205 bp, Lycopersicon esculentum
ttaatcagtgcatcagactaccggatgaaatcacaggaatgaaaccaaagggaataggagctgattttgctaggggatacttgagtaatgtatgcgttgctggagaattgcaaagaaatggcctcggctacgctcttatttgtaaagcaaagacggttgctaaagacatgggaataagtgatctatacgtccatgttgccatcga

SEQ ID NO: 255 - DNA, 168 bp, Lycopersicon esculentum
tcgactgcccttgctggattcaatagtatttttgctacctgcagaaacttgagaggacaaaatacttgacgattacttgtatcggaaacgtggttgtgccttctgaacggacacccttaccgctactcttgaaaaccgcgactgacttattactacaaacttgattaa

SEQ ID NO: 256 - DNA, 160 bp, Lycopersicon esculentum
ttaatcccagtcaatgcccttacttcatttatgaagcttttgggatgtgtattctgaaatgaagaatgaagtctcttcacagctatatctcctaataatggaaggtttaccttgtaaacacttccctgacctccttgcccaatgcaaaattttgcatcga

SEQ ID NO: 257 - DNA, 305 bp, Lycopersicon esculentum
ttaaggggaatcttgacatgaaacagtggacactatatatagacattacaacagcctatgtgagaaaattattagctccaaaacgaaaagggaaagggacttcacctgttatgaaacatggagattttggatatcaattcgcaatgacatcagtttcttctcaataattgtcaattcttctgcagtctctaaaagttcatccagtctgttttgagctctaacttgatacggcttactgatgagttttacaaagtcctccacattactagtcgtgtccaagagctccttctgcatagcctcttcga

SEQ ID NO: 258 - DNA, 144 bp, Lycopersicon esculentum
ttaatcgactgtttgtagggggtgacagtgctggaggcaatatagtttataatatgatcatgagagcggggagagaaaaattgatcggagatgtgaaaattttaggtgcaatacttggatttccttatttgatgataccatcga

SEQ ID NO: 259 - DNA, 94 bp, Lycopersicon esculentum
ttaatggatatatactggaagtatgagacccgacccacttgtgaatcatccgaatcttgttccccatcggagtacctccgtcaccagaaatcga

SEQ ID NO: 260 - DNA, 124 bp, Lycopersicon esculentum
ttaatatggggtggagtgactcaaacttggtaagtttttcatataaattttttcttagttagaagaaagtaaacagagtcggagtcaacatttgagttgctgattctaaattcgggtgaatcga

SEQ ID NO: 261 - DNA, 92 bp, Lycopersicon esculentum
tcgatcccattcctactatcttgcgaacatcatgttggagaggtttgtgtctgtatgacgggtctacacctttaagtagactatctaattaa

SEQ ID NO: 262 - DNA, 116 bp, Lycopersicon esculentum
tcgatattgtaaacatttagtgtgtttgttacttcattagcattggtgcaagataacgtctagaccctcttgtttgtacttctctcttcagagaattgttattacgaataaattaa

SEQ ID NO: 263 - DNA, 245 bp, Lycopersicon esculentum
ttaattgcaaggcccaaatgaccaaatgcacctgccgtagaagccactgagcccactgccgtggaaatgggaacattgaatttagctgctttatctattatatcacccacattacatgttacaccaaccacatcaccaaacctattcattttttctagcgcaccacggagacgatgctccgacgatgggtctgaatcaacaataccgcgaggatccttgaggtacctgtacaagacgtagcaaag

SEQ ID NO: 264 - DNA, 181 bp, Lycopersicon esculentum
ttaacttgattcagcaataacttagctctcttctcattatcaaattcactagtctgaatctcttttagagtatccaaaacttcaaccatagtaggcctcatgtccctatctgtctgtaagcatagaaaagccaactctgctactgaactagtcatttcccaaatattggtatctgaatcga

SEQ ID NO: 265 - DNA, 132 bp, Lycopersicon esculentum
tcgaatgtatgcaaactctcagtgaagtgaacgagaagaaatcaaggatcctccagttctcctgaatgtgtgactttggatttggtttcaatgcacgaatcgtgtcagggaacgacttagcatcacgattaa

SEQ ID NO: 266 - DNA, 125 bp, Lycopersicon esculentum
ttaatgagctctctaccgattataacacctgcacatacatgttctgcatacttgattttcctctctatttcaggtccagttcccagtccaagaatatccaaagattccaaagttgatttcttcga

SEQ ID NO: 267 - DNA, 201 bp, Lycopersicon esculentum
tcgaagttgaaccctccgagctaaatttctccgagttgaaccagaagttgacataccaagtgacattttccaagacaactaatagctcaaaccctgaggttattgagggattcttgaagtggacttctaataggcactcagtgagaagtccaattgcagttgtgtctgcctagtcaaaaattggctatataagtgcattaa

SEQ ID NO: 268 - DNA, 163 bp, Lycopersicon esculentum
ttaatacacaggttgctgatttcgggctgtcaagggaagaaatggtcagtaaacatgcatccaacatccgtggaacattcggatatcttgatcctgaatatatatcaactaggtccttcactaagaaaagtgatgtttacagctttggggttttactattcga

SEQ ID NO: 269 - DNA, 111 bp, Lycopersicon esculentum
tcgatctgttgcttgtgatactgccatatctattccataatctcctttggttgagattgtgaagtgcgaaatgttcgatcctcttcgctatcagcgaacagagattattaa

SEQ ID NO: 270 - DNA, 145 bp, Lycopersicon esculentum
ttaatcaacaaaaggtgtaagaaataagagcttcgggaatgacatgcttgatttttcccattgataatggaaaacgaacttagagtaggatgtaagagttctgcagaacccagtcactttgatctaaaactgttgttgccttcga

SEQ ID NO: 271 - DNA, 97 bp, Lycopersicon esculentum
tcgacctgattgattacttccaatttttctacaaacactatgtctcagtcccaaacaagttgggcttggctatatgaatactcacttctttatttaa

SEQ ID NO: 272 - DNA, 103 bp, Lycopersicon esculentum
ttaacacttaccccggtgagtctcgcctggtttcactgcatcgccggagctgctggctgcactgccttgctagttctctccgattattggtgcttactgtcga

SEQ ID NO: 273 - DNA, 101 bp, Lycopersicon esculentum
ttaagatccgtacaacacctgcaagtatgtgaggacttgaggaaagtaaggaagtagaggccttgcatgaactagatcatgatgatgatgatgatggtcga

SEQ ID NO: 274 - DNA, 76 bp, Lycopersicon esculentum
ttaaagagcttgttcaaacaccaacaaggagtgtagctctcatcccaagggaaaaaatcccttgcaaactcgtcga

SEQ ID NO: 275 - DNA, 112 bp, Lycopersicon esculentum
tcgacggcctgtgggcattcagtctggatcgcgaagactgtggaattgatcagcgttggtgggaaagcgcgttacgagaaagccgggcaattgctgtgccaggcagttttaa

SEQ ID NO: 276 - DNA, 112 bp, Lycopersicon esculentum
tcgacggcctgtgggcattcagtctggatcgcgaaaactgtggaattgatcagcgttggtgggaaagcgcgttacaagaaagccgggcaattgctgtgccaggcagttttaa

SEQ ID NO: 277 - DNA, 259 bp, Lycopersicon esculentum
ttaaagtggtacgcgagctgggtttagaacgtcgtgagacagttcggtccctatctgccgtggacgtttgagatttgagaggggctgctcctagtacgagaggaccggagtggacgaacctctggtgttccggttgtcacgccagtggcattgccgggtagctatgttcgggaaagataaccgctggaagcatctaagcgggaaacttgcctcaagatgaaatttcactggaaccttgagttccctgaaggggcgtcga

SEQ ID NO: 278 - DNA, 233 bp, Lycopersicon esculentum
  misc_feature (28)..(): n can be a, c, g, or t
ttaaagtggcaattttattcaggcattntattcatctagaaaacaagaaagcctattctattgcctttattcatacaactggatgcatttattttctcaaacatcatgagcccctaatatgcttagccttcaccatcttgactagtccctcagcacccattccaccagagttgaaacaccataacccgtggcgtttttcttttcatcgctccatacggggccagctacgtcga

SEQ ID NO: 279 - DNA, 91 bp, Lycopersicon esculentum
ttaatgctgccgcacaactccaatgcaactgctccgccacgctaactgattccgatttgcaacaattggatactactcctactatgttcga

SEQ ID NO: 280 - DNA, 78 bp, Lycopersicon esculentum
tcgaccccgaactctttgctcaacttgattatgttttgatgaatgtctccataatctcatggaatgtgaggggtttaa

SEQ ID NO: 281 - DNA, 60 bp, Lycopersicon esculentum
tcgacgagaaagagacaggaagccatttttttgtgtgtgggggggggagataaaaagttt

SEQ ID NO: 282 - DNA, 145 bp, Lycopersicon esculentum
ttaactaagtctctgaaagcaacaagagtgattacaatcttgtcttgtaataattatctatttggttttagcgaaaaatatcttctgctaaatgaaactggagttgaaattgttcatttcatgtagggtatgcccattatgtcga

SEQ ID NO: 283 - DNA, 114 bp, Lycopersicon esculentum
ttaaccatttgctctcttgactgcacagttgcaagacccatgcttcggtaccttgcagctaatgcagcacttatagccacatcattggtccgtgtaggcctcagtttctgtcga

SEQ ID NO: 284 - DNA, 214 bp, Lycopersicon esculentum
tcgaccatcatgatttcatgactgaggtgcacatttggaggcagacatgacaaaaccatctcaagggacctcctcttgtttatcctttatgggctacttgcacttttcatgaatatatgtaatcattttgacttcaattgtttctagtttccatctgttttagcatattcagctaaactaacatttgcaacaccaatctgggtttttgcgttaa

SEQ ID NO: 285 - DNA, 122 bp, Lycopersicon esculentum
tcgaccaaaggtgatcctattacaggaatagcgtcagggacacctgttactattttcactgcccaatagccaatttggtcccaaggtaaggaataaccagttacgccaaaagatgcggttaa

SEQ ID NO: 286 - DNA, 168 bp, Lycopersicon esculentum
tcgaccgatagtgaaccagtaccgtgagggaaaggcgaaaaaaaccccgggaggggagtgaaatagatcctgaaaccgtgtgcatacaaacagtcggagcctcttcgtggggtgacggcgtaccttttgtataatgggtcagcgacttacattcagtggcgaggttaa

SEQ ID NO: 287 - DNA, 236 bp, Lycopersicon esculentum
ttaacctcagaacgattgacaagggcctgaagagctgtaagacctgcaacaataagacctgcaccttttgcagctgatactttagcgagtcttggaacagtcaaactctcattagccaccgcatattcagccgatccacctccattcatggcgttgagcatagccacaaccttgttaccacccttgaatctttttacattagatcctacctctacaaacctctcctcccacgtcga

SEQ ID NO: 288 - DNA, 242 bp, Lycopersicon esculentum
tcgactaatacatgaccagcatcagctttcacagcatccccttcaacatgaggaggttcatctgtgggcttgtagattccagcgctgctgataaacaaaaattgcttgacaccagaactcttggcccagtcagctacaggacttacagaatccaagtctttaccattgttgtccaaaacagcatcaaatacttcaccctctaaaatctttccaacatctgcaggattaccccatacggttaa

SEQ ID NO: 289 - DNA, 150 bp, Lycopersicon esculentum
ttaaactaatcaaatgtttgaaagccataaaggaggatgccttctcattttctgaatatcctgttatactcacttttgaagaccaccttcatccttatccacaccttcaagaaaaagtagctcagatggtgaagagcacatttgggtcga

SEQ ID NO: 290 - DNA, 95 bp, Lycopersicon esculentum
ttaatagttctgctgctgaagaagtggagaatttgcctgttgtatcaaccagtgtagcttatggtgtttatatggcagtttccagcaaccttcga

SEQ ID NO: 291 - DNA, 172 bp, Lycopersicon esculentum
tcgacttctgcactggagagctgtgtgacggcaagcttgtgcgactgagagctgcgacaccctttggtctctgttgctgccgaactctgcgacctgctgctgaggctgcgccggtagtgaatgatcatgcaagagaacttattgaaacaaaaagctcaaatagatcggttaa

SEQ ID NO: 292 - DNA, 177 bp, Lycopersicon esculentum
ttaagatcaggaaatattctaagggtcaagcaggcaaattatggtcaaagctagcttttattagtaggtagtatgagaagatttaggagctcccttcaggcacttcactattcagttggcaaagaaatggagttgcgaaactagatagtatgcggaagtacttctatctggtgtcga

SEQ ID NO: 293 - DNA, 85 bp, Lycopersicon esculentum
tcgacaccaagtgtatgggtcccaaggattgtctctacccgaaccccgacagttgtacaacctacatacagtgtgtaccgcttaa

SEQ ID NO: 294 - DNA, 140 bp, Lycopersicon esculentum
tcgacaacaatcaatttgttctgattttcatccaccgcataacccaacaagttcaccaactgaggatggtgcactctagaaagaatctcaatctcattttccgccggagaattactagaactgttacggggttcagttaa

SEQ ID NO: 295 - DNA, 201 bp, Lycopersicon esculentum
tcgacaggcataccaggtttccatccagaaggcactacgatatttggagggacaaggccctgaattgctgaaagatttgcaagtgagttggtgtctgcaagtggttcaatgagtggctcaacaatcttcttgccttcatctgttttaggtaaaccaacatcaagatcagcaaagatatataattgagatgcacgcagttaa

SEQ ID NO: 296 - DNA, 201 bp, Lycopersicon esculentum; identical to SEQ ID NO: 295

SEQ ID NO: 297 - DNA, 199 bp, Lycopersicon esculentum
ttaacttggttcctctggaggataagtttgataacaccacagctatgctgatctatgaaagattctggaaaaagaagatgcctctcaatagaacgatgtttggggattatgatgtgatgcatattatgtatccagggctgcctttttctccaccgtctggcattggttctggtaacgggccaactggaagtgctgtcga

SEQ ID NO: 298 - DNA, 153 bp, Lycopersicon esculentum
ttaagagctgagctaactaaaccattacaacagctgcaagatactgcaagaagaatagcagagatacaacgtgaatgcaagttggagataaacattgaagagtatgtagaggcatcagtacggccattcttgatggatgtcatctactgtcga

SEQ ID NO: 299 - DNA, 103 bp, Lycopersicon esculentum
tcgacccccatcagaagcagaaaggcttggaggatttacagtagatgaatccgcatataaatgatctttgtcatcaagatacaaggcaggaggatgatcttaa

SEQ ID NO: 300 - DNA, 104 bp, Lycopersicon esculentum
tcgaccacttcgccccgtctatggtgaagatggggaacatatctcctttgacaggttccagtggggaaatcaggaagacttgcaggaagatcaactcatcttaa

SEQ ID NO: 301 - DNA, 272 bp, Lycopersicon esculentum
ttaatgagatgttcaaatccggtgtggctttggacgctgtaacattcaacactatgatctttatttgtggaagtcatggctacttggaagaggcggaagcattgctgaataagatggaggaaagaggaatatctcctgatactaaaacatacaacatcttcctatctctttatgctaatgcagctaagattgatagggctcttcagtggtatagaaagataaggaggacgggactttttcctgatgctgtgacttgtaaggctattattcga

SEQ ID NO: 302 - DNA, 186 bp, Lycopersicon esculentum
ttaaaaaccgtcaacatccatgtagcattctcaacttcttgaggattagatttgacataacgatgattgaaggtacagccatcatgcatatcaactttgtgatcatcttttaggtgtgcaacgaggtactgaatatcaccggtaacagcgcattcagatcctgcatatgggcaattgtatggtcga

SEQ ID NO: 303 - DNA, 102 bp, Lycopersicon esculentum
tcgaccccaagcctattccgggcgactggaatggtgcaggtgctcacacaaattacagcaccaagtctatgagggaagacggaggctatgaaataatcttaa

SEQ ID NO: 304 - DNA, 101 bp, Lycopersicon esculentum
tcgaccatagaccacaactacatgtaacatacacaccatgctatccagaaccccccccccacctaaaacgaagtctattgtaaacaatgtctctatcttaa

SEQ ID NO: 305 - DNA, 140 bp, Lycopersicon esculentum
tcgacctcaatcagtgagttgtcttactgtgcacatgagaggaagctttatatcaccctgcttccttttcttgctggtgttgctattgctggcatcttactcaggactcatcgtcttattcttgcctacattgatcttaa

SEQ ID NO: 306 - DNA, 78 bp, Lycopersicon esculentum
tcgacccgacccatattcgtattctattccttgagcttcacagagcttaccgagtaaaccaccaatccaaccagttaa

SEQ ID NO: 307 - DNA, 179 bp, Lycopersicon esculentum
ttaagaagacttgtggctggagttggggttgaaaacctatttcatctgtactctcccaacctaaaggagataaaagtgttggacaagaaaagagtgaggagggccaagctatactatctcagggacaaaatgaatgccctgaaaaaacactaacaagctagctcatgtggaagggtcga

SEQ ID NO: 308 - DNA, 93 bp, Lycopersicon esculentum
ttaagaaccaacaggatgaagttctctacaacccttgtgtttgtgattttagccttgttgctcactacaacttatgcagaacaatgtggtcga

SEQ ID NO: 309 - DNA, 266 bp, Lycopersicon esculentum
ttaagacgggaataataaatttcatttgaaaaggtaaagtggcaattttatttaggcattttattcatctagaaaacaagaaagcctattctattgcctttattcatacaacttgatgcatttattttctcaaacatcatgagtccctaatatgcttagccatcaacatcttgactagttcctcagcacccattccaccagagttgaaacaccataacccgtggcgtttttcttttcatcgctccatacggggccagctacgtcga

SEQ ID NO: 310 - DNA, 183 bp, Lycopersicon esculentum
ttaagaccaacaatgagaaatgtagtcaaaatgctggaaaatgcagaaccttgcagattagttgggataattgtaagcaaagatgatggtagtaacaagacagaacaattgaaggatcacaccaagatgtagcctttgttcgtttcactcttcaacctgatctctgtcctcatggaacgtcga

SEQ ID NO: 311 - DNA, 182 bp, Lycopersicon esculentum
tcgactcccacgagtacggaatagaaggcatcgtgtgttagacgagtttgtatttgtgtttttcccataacattattatggtcagagccaagagttctcgtccatcggctttcgtgaaattatctactgctgtatgattgatacttttatagagtaaatagaagtaatgagtttccagttaa

SEQ ID NO: 312 - DNA, 204 bp, Lycopersicon esculentum
  misc_feature (9)..(): n can be a, c, g, or t
ttaatgcantacaagaccatttatcctgccctgttgaggaacctgttgcagagccccaaaagcatacttatgcttctattctacaagttactaaagggaattctgcacaaggactgggacagtcttctctcaacaaatctacacctcctccttcagagtggcaacatgtgccacagcctcctgctttgccatcagttcattcga

SEQ ID NO: 313 - DNA, 123 bp, Lycopersicon esculentum
ttaaactgcaagtatgctatatgacttttgatagcacatacgaagttgcaacagcttgtgccttagctctaggagcagaaaaactaatttgtatcataaacggcccaattcttgatgagtcga

SEQ ID NO: 314 - DNA, 155 bp, Lycopersicon esculentum
tcgactcaaaatttcctctgagcaaggctgaagatgcttggagcaagagtattgatggacatgccaccggaaagattattgtggagccatagagcagtgttctcccaaattatgtaataaaatgtttgcgaggcttgtgcagtatgaccagttaa

SEQ ID NO: 315 - DNA, 153 bp, Lycopersicon esculentum
tcgactccagatttcctctgagcaaggcagaagatgcttggagcaagagtattgatggacatgctaccggaaagattgttgtggagccatagagtactggtctaaaaaggcttgtgtgcagtttgtcatctggctaggaaaccaaaaagttaa

SEQ ID NO: 316 - DNA, 175 bp, Lycopersicon esculentum
ttaagggtttccggaatgctgccgaaccaaggatttggtgaactagacagattgcgacatagaagtcccagtcctatgggttcagcaaaccttatgtcaaatgttactggagcaggattgagtggttggaatggacttccccaggagaggttgagtggacctcctggaatgtcga

SEQ ID NO: 317 - DNA, 93 bp, Lycopersicon esculentum
tcgacccccgggtatagaaaagatcatgttgggagcagaatgggccctgcaatgcacaaattgggattagtcgggctacaatgtatgtattaa

SEQ ID NO: 318 - DNA, 104 bp, Lycopersicon esculentum
ttaagggggtgttctctttgtaataaaaagagaagaaataaagttgttgtaacaatatataatgcctatctatttggttgaggttttatgggtttttgggtcga

SEQ ID NO: 319 - DNA, 288 bp, Lycopersicon esculentum
tcgaccggcatgagcttacggagcaagtccttcaccctcaccggcgcaccttctcccgaagttacggtgccattttgcctagttccttcacccgagttctctcaagcgccttggtattctctacccaaccacctgtgtcggtttggggtacggttcctggttacctgaagcttataagcttttcttggaagcatggcatcaaccacttcgtgttctaaaagaacactcgtcatcagctctcggccttagaatcccggatttacctaagattccagcctaccaccttaa

SEQ ID NO: 320 - DNA, 212 bp, Lycopersicon esculentum
tcgacccctccaccattctggaaagaaatgggaagtagtatctgccacatgatagagttagcgtatcaattatttcggaatattgtgagagaagagcaataatttggtgttcaatttgcatattgtcttcatatctcataacaatagagaataaaagaagagaaaacagactgttttgtactttggattgtatctggaaaattgttacttaa

SEQ ID NO: 321 - DNA, 142 bp, Lycopersicon esculentum
ttaagtcccaatatcaagtggagaaagataccatgggcttgccgatgaaaagcaaaacatatgggcacgaagtgtgttgaacatgagacatgaataccctccaattacaagatgtcaattggcgccaagatcttggagtcga

SEQ ID NO: 322 - DNA, 80 bp, Lycopersicon esculentum
ttaagatgtgcttttggagcactatacctttcatagaccgatgcacgcctcctacttattacgcgttcaatatgagtcga

SEQ ID NO: 323 - DNA, 271 bp, Lycopersicon esculentum; identical to SEQ ID NO: 1

SEQ ID NO: 324 - DNA, 84 bp, Lycopersicon esculentum
tcgactagtgatttgtttttttgacttgatttggtgttgaaaaaaaattcctactatatttggtttgggttggtttcaacttaa

SEQ ID NO: 325 - DNA, 105 bp, Lycopersicon esculentum
ttaagggcaatccaagtcggtctattatgcgtgcaacaatgtcctgaaaataggccaaatatgtcttctgtcattatgatgttgggtaacagccatgggagtcga

SEQ ID NO: 326 - DNA, 91 bp, Lycopersicon esculentum
ttaatgatgaacggtatctaaagaatgtgttttggattgattccagatcaagagcggcatatggttattttggtgatgtggttgtggtcga

SEQ ID NO: 327 - DNA, 192 bp, Lycopersicon esculentum
tcgaccaatcatctatggtgatgtgagccgccctaagccaatgactgtcttctggtcctcaaaagctcaagagatgaccaagaggccaatgaagggaatgcttactggccctgtcaccattctcaactggtcttttgtcagaaatgaccaacctaggtttgagacctgctaccagattgctttggccattaa

SEQ ID NO: 328 - DNA, 222 bp, Lycopersicon esculentum
tcgacccgaagctccgcaagtactacagaaattggaggagataaaaccagagatgatgttagcagctactagttctggagatgatggcacaacagttcaagaaaagagtgatgaataaactctgctgcagttttggaatttttgagcttttcacttgatatctatttgtttattctttcatttatttttacagttagtagtagaaatgttgtttcatattaa

SEQ ID NO: 329 - DNA, 133 bp, Lycopersicon esculentum
ttaatatctttctcggcggtctggagacgggtaaacattttgacagtgaaggactctgtataaaattgttttatatgctttttacaggctgacataaacttttcaggaccaatttcattttcatatgggtcga

SEQ ID NO: 330 - DNA, 132 bp, Lycopersicon esculentum
ttaatattcctgcaccgtcgtatgatgcgatggggggacggatcgcggaaggttgtccgactgttggaatagacggtttttgactcatagaaggtgcttatgcaaatccgggcgcgtaattcaaggggtcga

SEQ ID NO: 331 - DNA, 276 bp, Lycopersicon esculentum
tcgacctgctatgaagagtcactttctatggttagaggggaattcaattgaagaaatcaccatcaaatgacattgaaatggatagttttgaagttattgttcatgaggcaagtggggtcaaaagttcaaaaagtattctattattgctcaagggattaggaacaaagttgcatcttgatgcccagattcccctttcacaacaaattgatggagtaatacaaaacattgtaagtggcaaatgagcgagttgaaatggattgagctgtgattgattaa

SEQ ID NO: 332 - DNA, 144 bp, Lycopersicon esculentum
tcgaccttgcatatccagtgagagccagccaaagtgttgcaatgttatttgaacccctcatgagataatacaatcctccaatggaacacaaagcatacatactctcagtatatattgttgagtagaatatggaggcgggattaa

SEQ ID NO: 333 - DNA, 124 bp, Lycopersicon esculentum
tcgactatgaataactccggagccctgacaatcaaaaacaaactgaacgggtaacaccggagacgcggtcgaagtacccaatcaaaatcaactatctagaccatggctcggaataagagattaa

SEQ ID NO: 334 - DNA, 236 bp, Lycopersicon esculentum
ttaaccaactcaaaaaccatcaaatgcagatgtgttggcacatagggaaatgtaaagtttgggtccccataaatgttgaccttaggtgcacttccatattcgcgcaagcaaatagaacgggcatcctctgtagcgttgcgtgcaacctccaagggggacatttttgtatgtatatagcccacacaatcaggaggtggattaggatcgtgtagagcgacatgctgccctgtgttcga

SEQ ID NO: 335 - DNA, 271 bp, Lycopersicon esculentum
tcgaataagacatcaagaggctgatccttagtaagagccaaacgccttcttccatcttggaaaactacacccacatttacacataactgacccttacggcaaaatggacagaaaataggagtaggatcgcaaaggactaagttgtaaccaagttttgatggaacaatcttgaaatagctgctatctctttgccctatgcttcctccagtctccaataataccccgcctagagtttcattgtaatctcccactttccaaattgtatatttaa

SEQ ID NO: 336 - DNA, 146 bp, Lycopersicon esculentum
tcgactaggaaatacttggattcatggagcgctttcttatgggcaacaacacctacacttttttactcgttcacattgagactatatactttgaagggccatcaacttgatgcatcgacggttgttacttgtggcgcactgtttaa

SEQ ID NO: 337 - DNA, 106 bp, Lycopersicon esculentum
ttaatgaaactttagccttagctggtcatgaccacgatactacatgtttcgttaggtgggccaggaatgcccgacaaatcaatttatctggtaaactactagtcga

SEQ ID NO: 338 - DNA, 79 bp, Lycopersicon esculentum
ttaatggtgataactatcagatttgggcggtaaggatagaaacatatctagatgccatggatatgtgagaggcagtcga

SEQ ID NO: 339 - DNA, 403 bp, Lycopersicon esculentum
tcgactggacatgattttgcatcccaggccttcctatatatgcttgagattgttgaggttgtgagggaggtggtaaggtggcattttgctgcggttgaatatacccatgcaaactttgaggcaccacattaggaagaccttgtgggcgaaattgagactgcaacggctgctggtgagtaaaatgaccttgctggtgagattggcttggaaatggaccaggaaatgtatgatgactgtactgttgagacattggctgttgattagactgaccaggatgttgctgaattgagtgtcctgcttgctgagcaataggataaagttgagattgtgcaggaacttgaccttgtgaaggtaccagccccggttgctgattgccgattagacccagaggtgggcgcattaa

SEQ ID NO: 340 - DNA, 343 bp, Lycopersicon esculentum
ttaatagcatacgcagccgacgaggctgccactacacaatacacaatgatgttcacaaccgacaaggctgcacttgtacaaatggacatcccaaacaaaccatgtccagaccattatccaaaacataattatacaacccacacgatgtctacagacctctaagagtatagtagtgaaacttgacgggacagggccccgccatacccatagacaagcaaaagactacataagaagttatgtaccaaaacgactgggctccggaacagtggagctctcccaaacagcagagtagatatcctaggcgggaggatcaccgaactgagcgtctacaccgaatagtcga

SEQ ID NO: 341 - DNA, 230 bp, Lycopersicon esculentum
tcgactcagcagtagatcaagccagtcttcactccatcggaaattcagggtgagctaatgcgtctagcttggactgggtcttcttcttcaagtcttgaagccttgaacttccggcatggactagatttttcgttatttttagcttcttagatattcttagatagtttagtttagtaatttgattttagatgttcttgtgatgatgacttccagattttggggaatattaa

SEQ ID NO: 342 - DNA, 115 bp, Lycopersicon esculentum
ttaatatgacgaccggtgagggacttaacgaaagactgcctcttttgcaaacagccttgtaaagcaatcaactgtatatgttatttttgactttccatatcagtttccaagtcga

SEQ ID NO: 343 - DNA, 169 bp, Lycopersicon esculentum
ttaatgatccaacttttgctttaccttactggaattgggatcatccaaaaggtatgcgtatacctcccatgtttgatcgtgagggttcatctctttacgatgagaaacgtaaccaaaatcatcgcaatggaactattattgatcttggtcattttggtaaggacgtcga

SEQ ID NO: 344 - DNA, 79 bp, Lycopersicon esculentum
tcgacgacccatctgaaaatcttgctgaattttgagttatggctgttgaaattgttgattcggatgaaaacccgattaa

SEQ ID NO: 345 - DNA, 71 bp, Lycopersicon esculentum
ttaatcggcaactaatcgtcggtggccggagcgggcgacgacggtccctttaggtgcaatttttccgtcga

SEQ ID NO: 346 - DNA, 97 bp, Lycopersicon esculentum
ttgggtggaaatttcttttaccgatttctctaggtaatctattattgacaacctcgttccaacttctttcactgtaaaagaccacaatatcctttaa

SEQ ID NO: 347 - DNA, 261 bp, Lycopersicon esculentum
  misc_feature (244)..(): n can be a, c, g, or t
ttaattatgtacagagaaaaggtacaatgttcatggtgaaatggtattcacagatagagttctatttcctcttccacaagtatcatactgttccattggacaaatctaaactaggtatttcattacaaggacgaatgcaatacaactacagtatcaatctccatttgatatccctatctcagagtccttggcaggctgggaatttatgtttgtcagcggactagctacgctcccacaaactgangctgcagttgctgtcga

SEQ ID NO: 348 - DNA, 76 bp, Lycopersicon esculentum
tcgaccacagaaaaggatgagctttatctaaatttggttctagaatttgtacctgaaactctctaccgtgtgttaa

SEQ ID NO: 349 - DNA, 261 bp, Lycopersicon esculentum
ttaattatgtagagagaaaaggtacaatgttcatggtgaaatggtattcacagatagagttctatttcctcttccacaagtatcatactgttccattggacaaatctaaactaggtatttcattacaaggacgaatgcaatacaactacagtatcaatctccatttgatatccctatctcagagtccttggcaggctgggaatttatgtttgtcagcggactagctacgctcccagaagctgaggctgcagttgctgtcga

SEQ ID NO: 350 - DNA, 96 bp, Lycopersicon esculentum
ttaattcccctttgcatgaacgactatatcgtcattctactctcaagatttcaactcggatctctcaccttcccaaacatcatattgttgtgtcga

SEQ ID NO: 351 - DNA, 95 bp, Lycopersicon esculentum
ttaattcataacattatgcagcaacctgatgttagaaaaacatcaagaatttacaatctgagcacgaaatcattcacacgctggtttcgtgtcga

SEQ ID NO: 352 - DNA, 155 bp, Lycopersicon esculentum
ttaattggtcatactgcacaagcctcgcaaacattttattacataatttgggagaacactgctctatggctccacaataatctttccggtggcatgtccatcaatactcttgctccaagcatcttcagccttgctcagaggaaattttgagtcga

SEQ ID NO: 353 - DNA, 124 bp, Lycopersicon esculentum
ttaattggtatgaatcttgggagttatctaggggtgagggtgctcctccaccggtatgggatgaatttgtggaggctttccagggccacttcctgcctccagagatgaggcgagctatagtcga

SEQ ID NO: 354 - DNA, 155 bp, Lycopersicon esculentum; identical to SEQ ID NO: 352

SEQ ID NO: 355 - DNA, 77 bp, Lycopersicon esculentum
tcgacgcttgaaggcaaatgacattgcatttgaaggcaaggaaatgctaggaataaatgtcatttgctcatctttaa

SEQ ID NO: 356 - DNA, 197 bp, Lycopersicon esculentum
ttaaatactacattcagactaagaagacttgtggctggagttggggttgaaaacctatttcatctgtactcttccaacctaaaggagataaaagtgttggacaagaaaagagtgaggagggccaatctatactatctcagggacaaaatgaatgccctgaaaaaacactaacaaactagctcatgtggaagggtcga

SEQ ID NO: 357 - DNA, 135 bp, Lycopersicon esculentum
tcgaaatcggaacactactggaggaaatatgagggtctaaagcccatgtcaaaagctcatcaccacagaagtcacccgccttgagatgaacagagttgaaaaaccactccttccaccatatgtgggcgtagttaa

SEQ ID NO: 358 - DNA, 128 bp, Lycopersicon esculentum
tcgacccggtttacccgggtatacttgatccacaaactcccctttagcgaataacccggtaaaaattattccccatgcaccacaacccccatgtaattgtgctgcttcaagtggatcgtcgtatttaa

SEQ ID NO: 359 - DNA, 127 bp, Lycopersicon esculentum
tcgaacatgcttactgacaatatgacgacaattgatctgtcatacaacaacctcaacggaactattccttcaaacttttcaagccttccacatttgcagaaactgtcgctagaaaacaattcgttaa

SEQ ID NO: 360 - DNA, 37 bp, Lycopersicon esculentum
tcgaaactgacacgtgtcccttcctgttgaacgttaa

SEQ ID NO: 361 - DNA, 133 bp, Lycopersicon esculentum
tcgaagcaacgcgaagaaccttaccaggccttgacatccaatgaactttccagagatggattggtgccttcgggaacattgagacaggtgctgcatggctgtcgtcagctcgtgtcgtgagatgttgggttaa

SEQ ID NO: 362 - DNA, 120 bp, Lycopersicon esculentum
tcgaatcaactcaggatcaaacacttgtgttgtccccctctcttgaactatacatttcacccatttaggcaggtcaattccctcgtcgttgatgacactactaggatttttacccgttaa

SEQ ID NO: 363 - DNA, 405 bp, Lycopersicon esculentum
tcgaaacggggctaacaaacttacctagccttgccacccactgaactttataaagatcgatatgagcctaaggtaacattgggacacgtgctttatggctgttttcaccacacagtttagagaggtctattttgcgcatctaatcttgaaccaccaacgccagtttaaaacacctctttgttaagagcgccactttagcggccatataaaacaccccgcgcacataaaaatattgtgaaatttcgcctccgtgtgttaaaaaacactagtggtctcttatggtgtggggaaaaaatccctcagcgggcgcgtataactctgttagaacgattacctgttagccaactacttcctctttcccgtctctctctctcttgtctattacgtcaccgatattaatattaa

SEQ ID NO: 364 - DNA, 395 bp, Lycopersicon esculentum
tcgaaccgtatggggtaatcctgcagatgttgggaagattttagagggtgaagtatttgatgctgttttggacaacaatggtaaagacttggattctgtaagtcctgtagctgactgggccaagagttctggtgtcaagcaatttttgtttatcagcagcgctggaatctacaagcccacagatgaacctcctcatgttgaaggggatgctgtgaaagctgatgctggtcatgtattagtagagaaatacatatcagagatatttggtagttgggcaagtttccgtcctcagtacatgattggctctggcaataacaaggattgtgaggaatggttctttgatcgtattgttcgtggacgaccagttctaattcctggctctggaatgcagttaa

SEQ ID NO: 365 - DNA, 141 bp, Lycopersicon esculentum
tcgaagggctgacaagtagacgagtgatgaggtctctttgttgaagcctcgttctcactattttcatcacttttggcactgtttctttgttccaaataacttgcacaacaccataggcttgctcttattttcaatagttaa

SEQ ID NO: 366 - DNA, 294 bp, Lycopersicon esculentum
ttaaagatggtactcttttagcaccaaaggtattcaatgtgcaattggagggtgcattcaagagttttgatacagaatgtgagatgttgcgcaacctccgccaccgaaatctgaccaaagtcatcaccagttgctccaaccttgatttcaaagccttagtgctggaatacatgtccaatgggacacttgataaatggctatactctcacaacttgttcttgaatttatttcaaagattagatataatgatagatgttgcatctgcaatggtctatctccacaatgggtgttcga

SEQ ID NO: 367 - DNA, 168 bp, Lycopersicon esculentum
tcgaaccgaactcctttacgaccaggtggtggggttgtgtttgataccaccaccccgtacttgggctcgggtaagggtggaagcagtaccgcgacgttggttgcgcaggtcagtggtaagcccacgatgaacgacggcgcgtgcggtatcaagaggggcggattttaa

SEQ ID NO: 368 - DNA, 79 bp, Lycopersicon esculentum
tcgaacccttaaggaagaggattaaaatgtggaatgacatatagttgttgatgggttttgagccgtgttatatggttaa

SEQ ID NO: 369 - DNA, 79 bp, Lycopersicon esculentum
tcgaacccttcaagaagaggatgagaatctggaagttcatatacttgatgttcagttctgagccggcttatatgcttaa

SEQ ID NO: 370 - DNA, 191 bp, Lycopersicon esculentum
tcgaaaggaaagaaaatcaagttcttcaagtgcagatgatctaatcactccaggcatattcaatggtgcacaactatagtccatgtgcaacctggaaaacactcttttccaaatgcggtaaaaccatcaacagactttgaagcaaaagcggaggtcggaacatgaatgagcattgcatgtgattcccttaa

SEQ ID NO: 371 - DNA, 142 bp, Lycopersicon esculentum
tcgaactaggagcagccatagcacctttggcgtttctcaactttctcaccttgggtaaaggaccatcatcataatcttcatcactagaatcagaagacccatttccatatgtttccttcatacaccaatccgagcaaattaa

SEQ ID NO: 372 - DNA, 198 bp, Lycopersicon esculentum
tcgaaaacaggttttactatccgacccatttcttttgcttgatcaggaccacgagtcatgttcacttgttcgtttatagtatcctgacctctgactaaagatggagaagtagatgccgcccactccctataaaccggatttattcgggcattcatgtttgacttatttgtgcgacaaggcgtcatttcgcctaattaa

SEQ ID NO: 373 - DNA, 152 bp, Lycopersicon esculentum
tcgaatggagtcagcaataggctaccgcctcatggaactgtgatacttgtacttggtggtgtcaccgcgggtgaatcagcggcgctacaacgaaaccccgatatctcgctaattgcagctagcgaccttaccgatggccatcgccttattaa

SEQ ID NO: 374 - DNA, 293 bp, Lycopersicon esculentum
tcgaattcactactcacaggaacattggcatcactctcttttgcctcggaacacttcaggtatttgctttgcttctgaggccaaagccagaccacaagtacagattatactggaacatctaccaccatgctgttggatacgcggtaataagtctgagcatcgcgaatgtttacaaaggatttgatgcattgaatggacagaagaattggaagagggcttacactggtgtgactcatagccataggtgccattgctgttttattagaagcttttacttggttcattgtcattaa

SEQ ID NO: 375 - DNA, 175 bp, Lycopersicon esculentum
tcgaacaacaatggctaacttgttgcatggattcaattggaaattaggaggagatatgaggccagaagatataagcatggacgagatttatggattgactacgcacccaaataagcctgtatctgtgattatggaacctagacttcccctccatctttattagaactgactttaa

SEQ ID NO: 376 - DNA, 103 bp, Lycopersicon esculentum
tcgaagatatcattcctgggttgcagtaggcaatgtttcatgcaaatctatcaaaatagagtctgaaaaagtttttaccagaagtatttacagaagtgtttaa

SEQ ID NO: 377 - DNA, 131 bp, Lycopersicon esculentum
ttaacagcaaagtaacctaacatggtaacttgatacaaaatcaaggtagctatcatgcgcgtgtaaatatggggccacattcttccgtagctccggaatcaggcactcaaagtgtatgttccctcattcga

SEQ ID NO: 378 - DNA, 295 bp, Lycopersicon esculentum
tcgaaaacgatgctggatattgagttgaccacattatgtatattacggacaaaccaataacaagtccacaaccgtaacccatgagaaccccctgccaactgatcattggtgaatcttcttcttcttgttgttgatctagctccgctggagttgtcacttgatcatcaccgccacaatgtgttgagagtggaaatccgcgtaacccatcatttccttggtgcgaagtgttccccaacgaatcaaattgttttcctttggggatgcatccaaccacatgattgtgagagagatttaa

SEQ ID NO: 379 - DNA, 116 bp, Lycopersicon esculentum
tcgaagtagttccacagattatctaattgtagatccgaacaggcttctcaaaattgtgaccggtctggtgggcacaatatgatcttcttggaggacaatccgagtgctctatttaa

SEQ ID NO: 380 - DNA, 82 bp, Lycopersicon esculentum
tcgaatcaatggttccaactcaacaaattcttcttgtgttagcccttgcggaactaataattgtctatcaccatcccattaa

SEQ ID NO: 381 - DNA, 82 bp, Lycopersicon esculentum
tcgaatcaatggttccaactcaacaaattcttcttgtgttagcccttgcggaactaataattgtctatcaccatccatttaa

SEQ ID NO: 382 - DNA, 123 bp, Lycopersicon esculentum
tcgaacaaaccaatgtgagcaagaagttcaaagaataatttatttgcagaacattgcaaatcaactaccagatgcatttactaatcttccaaggattactaaatcgcatattccagctgttaa

SEQ ID NO: 383 - DNA, 319 bp, Lycopersicon esculentum
tcgaaccatatatcgtagtagaagtgtaagttgtgaccataccggtgtcgtggatcaatagcttctagccaatgctgcagagcaagtttctgagctttttcatcttttgacaatcctttaccaactttggcagctcgtgtacgtgctctagcccaccgcgatacagcagtttccggtttctccacgttgaagaacgaaacagaactccgtttgagagctgcgaaatccaaagccttccaccatagttcttcaaccacaacagcacaatctgcaagatttcttcttgtcctataactcttatacactttctgaagcttaa

SEQ ID NO: 384 - DNA, 294 bp, Lycopersicon esculentum
tcgaaacttcaagtggaacactaactcttccatttgcacatggcatggcgtaagttgcagctcagatggcactcgtgttgttgcacttcgccttcctggacttggactttatggtcctattccagacaataccataggaagactagatgcactaacaacactcagccttcattccaatgccctcactggaaatcttccttcagacatcacctctcttccctccctccgtttcatatttatacaacaaaacaaattttctggtgaaataccttcttctctatctctactccttaa

SEQ ID NO: 385 - DNA, 139 bp, Lycopersicon esculentum
tcgaatatgccaacacattactaaagcaagaaagagagagagaaatacatactttgagttggaactcaaaatgggttttggcctacataaagatgaagaaggaacagggaagtctcacaggcagcctttggtgtcttaa

SEQ ID NO: 386 - DNA, 102 bp, Lycopersicon esculentum
tcgaacaggcagacacaactcattgcatcagaacaaagaggatagacatctatagcaaccataaagcttcaagcacacagtgataccaactaacacccttaa

SEQ ID NO: 387 - DNA, 102 bp, Lycopersicon esculentum
tcgaacaaacagacacaactcattgcatcagaacaaagaggatagacatctatagcaaccataaagcttcaagcacacagtgataccaactaacacccttaa

SEQ ID NO: 388 - DNA, 238 bp, Lycopersicon esculentum
ttaacttggctacactacacgaacacgccatatttgtccaccaccaaggatcttctggatcttcactgctttccaatcccaaaaccactcccacatttgagaaacctcccactttttcttatggatccacctacccaaatactagaaaacatcccaaaccctcctaaaattacaccaacttctctcttaaccccccaccccaccctcttatattcacccttaaaaaaaactccttcga

SEQ ID NO: 389 - DNA, 211 bp, Lycopersicon esculentum
tcgaatagcactaccccaaaagaatacacatcagatttttctgtcaactgttgtcgcttatagtattcaggatctagatatccaaagcttcctttcacaacagtgctaacatgagtttttcctgatccactaagtggaccgattttggataacccaaaatctgaaaccttagcaacccatttatcatccaataaaatgttggttgacttaa

SEQ ID NO: 390 - DNA, 141 bp, Lycopersicon esculentum
tcgaatagctgggttgatttacgaagagactcgtggtgttctgaagatctttttggaaaatgtaattcgtgattccgtgacatacacagagcacgctaggagaaagacggttactgctatggatgttgtttatgcgcttaa

SEQ ID NO: 391 - DNA, 80 bp, Lycopersicon esculentum
tcgaacgtagtttgtatttgacagcttcacttgagtaaaaaagaacttgtcttcagtaaataaggaggcagataaattaa

SEQ ID NO: 392 - DNA, 168 bp, Lycopersicon esculentum
tcgaagatgttggaaaaccagtttcagagtactttggtgttagcggtgatgccccaagagttctcgcatatacaggaaatgaggatgggaggaagtttatactggaaggtgaaataaccttggatggtgtcaagtcatttggggagaagtttttggaagacaatttaa

SEQ ID NO: 393 - DNA, 82 bp, Lycopersicon esculentum
tcgaacgagcaattgaaaagattctggatcatcttcgggataaggatgatgcacacatcttctgtttagaagatcaaattaa

SEQ ID NO: 394 - DNA, 160 bp, Lycopersicon esculentum
tcgaaagcagcaaaacaaattagtcgttacaagttcccggcgcgtgtacgaaatcgctccttgtgagatcgtctttcgttcaaatcacacaaattagctgttacaagttcctagctggcagccatttgaattccattcatccaaattccccccaaattaa

SEQ ID NO: 395 - DNA, 167 bp, Lycopersicon esculentum
tcgaaagacaagttctttgttcatttttcccatctcatttactgatagagaatgtgatggtggaaaagccctgacatcatacagtaaatagaaggattagtaatgtaagacgccaagaccaaactccctttacttgtaccttctttttcagttttttccgttattaa

SEQ ID NO: 396 - DNA, 40 bp, Lycopersicon esculentum
tcgaccacattatactttgccctgatcttttacattttaa

SEQ ID NO: 397 - DNA, 107 bp, Lycopersicon esculentum
tcgaattcagcgggttggggaaatgcctgctgcttgtgttactgttatttgttgttacatcgtcccaacatagtagctagtcttagttgctcgttttcttgtattaa

SEQ ID NO: 398 - DNA, 127 bp, Lycopersicon esculentum
tcgaagaggtgaattattggacgaacaaaattggattcatcaatttcatttgccgaggatctccttcatccatccctatcaaatgaggggcagttgttgacacccattttgaccctccacaatttaa

SEQ ID NO: 399 - DNA, 125 bp, Lycopersicon esculentum
ttaacgagctctctaccgagtataacacctgcacatacatgttctgcatacttgattttcctctctatttcaggtccagttcccagtccaagaatatccaaagattccaaagttgatttcttcga

SEQ ID NO: 400 - DNA, 96 bp, Lycopersicon esculentum
tcgaaggtaactatcttcactacgaagggcttgaaacacaacagaattatgataatcaaccatatcagcacttgattgtgtaaatacttctattaa

SEQ ID NO: 401 - DNA, 95 bp, Lycopersicon esculentum
tcgaaggttgctggaaactgccatataaacaccataagctacactggttgatacaacaggcaaattctccacttcttcagcagcagaactattaa

SEQ ID NO: 402 - DNA, 60 bp, Lycopersicon esculentum
tcgaatcacaatggtaatcatggtaacctgagttgagagtggtagttgttttctctacga

SEQ ID NO: 403 - DNA, 155 bp, Lycopersicon esculentum
ttaattggtcgtactgcacaagcctcgcaaacattttattacataatttgggagaacactgctctatggctccacaataatctttccggtggcatgtccatcaatactcttgctccaagcatcttcagccttgctcagaggaaattttgagtcga

SEQ ID NO: 404 - DNA, 90 bp, Lycopersicon esculentum
ttaagatgcaccccctccgcgtcttttctcttctagaattgcagcctcattcaacatgtcagctgcatctttgtgcatgtctagcttcga

SEQ ID NO: 405 - DNA, 83 bp, Lycopersicon esculentum; identical to SEQ ID NO: 46

SEQ ID NO: 406 - DNA, 125 bp, Lycopersicon esculentum; identical to SEQ ID NO: 399

SEQ ID NO: 407 - DNA, 102 bp, Lycopersicon esculentum
tcgaacagacagacacaactcattgcatcagaacaaagaggatagacatctatagcaaccataaagcttcaagcacacagtgataccaactaacacccttaa

SEQ ID NO: 408 - DNA, 319 bp, Lycopersicon esculentum
  misc_feature (18)..(): n can be a, c, g, or t
tcgaaccatatatcctantagaagtgtaagttgtgaccataccggtgtcgtggatcaatagcttctagccaatgctgcagagcaagtttctgagctttttcatcttttgacaatcctttaccaactttggcagctcgtgtacgtgctctagcccaccgcgatacagcagtttccggtttctccacgttgaagaacgaaacagaactccgtttgagagctgcgaaatccaaagccttccaccatagttcttcaaccacaacagcacaatctgcaagatttcttcttgtcctataactcttatacactttctgaagcttaa

SEQ ID NO: 409 - DNA, 292 bp, Lycopersicon esculentum
tcgaattcactactcacaggaacattggcatcactctcttttgcctcggaacacttcaggtatttgctttgcttctgaggccaaagccagaccacaagtacagattatactggaacatctaccaccatgctgttggatacgcggtaataagtctgagcatcgcgaatgtttacaaaggatttgatgcattgaatggacagaagaattggaagagggcttacactggtgtgatcatagccataggtgccattgctgttttattagaagcttttacttggttcattgtcattaa

SEQ ID NO: 410 - DNA, 455 bp, Lycopersicon esculentum
  misc_feature (268)..(): n can be a, c, g, or t
ttaagggcaccccccctggacatacttctggaattccgcttattcaccccgcggtattcactgggggcaaagaggtgcaagataccaccaagcgcaaacttgcttataaagcccgcaacacgcgccgcacaccctcacactgagggggccctcttggcgctaaagagcgtggcaccacattccactggaccggggtacaacaacgacccgagggggaaaaccctggcgggcgatactatgtgggtccagagaaaaacacccaacctcngnnnnaaatcaagcagtccataggtcgtctcccggtgcgcaaaatgtgttacttcccgcccaggtcgcaatccaacctgatcagtcaggagcgcaccatcatactcagcctatgacgcatcctgtcctgcgactacagtcagataccctctacgtcaagccggacgaatctcacggatagtcctcga

SEQ ID NO: 411 - DNA, 211 bp, Lycopersicon esculentum
tcgaaatagtttgtactttgagaaagatggtataatcttcaggattcattgttccggtgattgtcatgtttgtaccatctagtactgaatcatcaggctgaaaaggtataatttcattctgtttttcgtttggcatatacttttgacatgacaaatttatggctggaggtaaatcttcatgttcatcaacagcaaggaatggatcccttaa

SEQ ID NO: 412 - DNA, 146 bp, Lycopersicon esculentum
ttaaaagatggaacagagatccgaaaagggcattgaaatttcagatcatacagaaaattataagactgaattggagaaaccgattcggaattcgtatatccaaacgctgtaaatacaaaacctaataccaatatgtatctaatcga

SEQ ID NO: 413 - DNA, 127 bp, Lycopersicon esculentum
  misc_feature (99)..(): n can be a, c, g, or t
tcgattgaagactagtgctagaaaaagttattgacgttgctgtgatggaatcagaaagatacaaaccttatttgttctgtttgaagtgaaatgtgcacntatnnncctctccctaaaatgtttttaa

SEQ ID NO: 414 - DNA, 180 bp, Lycopersicon esculentum
ttaaactgaacataaacattgacaatatcaaacaaaattattctcttagcaactgtgcctatgaatgcttccacactgatattgacaatatgaagtttggatctcctttcaaactccacctactttgtatcctaacccaaaagctggtactgtcactacaaatatacctcaggctatcga

SEQ ID NO: 415 - DNA, 179 bp, Lycopersicon esculentum
ttaaacttgagaagaaaaggactaactgatgagggagttcaaaagattgtgaaagatccagcaccatatggacctggtccatggggtggagaaggtggaaaaccatgggatgatggagttttcactgggatcaagcagataatcctaacacaaacatcagaggccatttcttgtatcga

SEQ ID NO: 416 - DNA, 170 bp, Lycopersicon esculentum
ttaaaaacctaagtgatgaaattcccttcttctttttttttcagttacaacaggctgacggccatcaacaaggaaatactgggctggagcttccaactgtgcagcccccaccacctatgcaggtgggggctagtccgggctcaatcagaccaggttctatggtggatcga

SEQ ID NO: 417 - DNA, 142 bp, Lycopersicon esculentum
tcgatggcagaagacagaattctctttatcaacaatcaaacatttcttctaatataacaggattacatgttgcttccagatataaaagaaatattgaagccctattgatattcatgtactgatcttcagtagtgtacattaa

SEQ ID NO: 418 - DNA, 105 bp, Lycopersicon esculentum
tcgatcaatgagtgattgtgcaaaccttgttagagcttgctcataggtttcaggattgtctttggaaactggtttcttcaataaatttttaccaacttgttttaa

SEQ ID NO: 419 - DNA, 234 bp, Lycopersicon esculentum
ttaatgaaatcgtgggagtaagactgatattcttgccgaattcatctgtgttcaccattcccggcgggatagtctgccagtacagttcgttgttctttcaattggtgatacgtacacttttcccggcaggaacatacggcgtgacatattcttcaaatggtgtatacccgccctgatgctccatcacttcctgattattgagccatactttggcgtaatgagtgaccgcgtcga

SEQ ID NO: 420 - DNA, 170 bp, Lycopersicon esculentum
tcgatgcacacatcaatggatgtaacgtgaagaaacaagtacaaagattatcaaaggatgaagaagtagtagttactacttatgaaggtatgcattcacatccaattgacaaatctacggataactttgagcacattttgagtcaaatgcaaatctataattccttttaa